(19) United States
(12) Patent Application Publication
(10) Pub. No.: US 2026/0076944 A1
(43) Pub. Date: Mar. 19, 2026

(54) COMPOSITIONS AND METHODS FOR PROMOTING BRAIN HEALTH

(71) Applicant: The Regents of the University of California, Oakland, CA (US)

(72) Inventors: Elaine Y. Hsiao, Los Angeles, CA (US); Elena Coley-O'Rourke, Los Angeles, CA (US)

(21) Appl. No.: 19/331,331

(22) Filed: Sep. 17, 2025

Related U.S. Application Data
(60) Provisional application No. 63/695,494, filed on Sep. 17, 2024.
(60) Provisional application No. 63/822,588, filed on Jun. 12, 2025.

(51) Int. Cl. A61K 31/417; A61K 31/191; A61K 31/198; A61K 31/404; A61K 31/4172; A61P 25/00

(52) U.S. Cl. CPC A61K 31/417; A61K 31/191; A61K 31/198; A61K 31/404; A61K 31/4172; A61P 25/00

(57) ABSTRACT

Disclosed herein are methods for promoting healthy neural development in a fetus, which include administering to a maternal subject gestating the fetus a composition. Compositions can include 3-indole sulfate, phenylacetylglycine, imidazole propionate, alpha-hydroxyisocaproate, 2-hydroxy-3-methylvalerate, alpha-hydroxyisovalerate, 1-methylhistamine, beta-hydroxyisovalerate, 2R, 3R-dihydroxybutyrate, or N-acetylleucine, or a combination thereof. Compositions can also include acetate, propionate, or butyrate, or a combination thereof. Also disclosed are methods for conditioning a female subject for bringing about offspring with healthy neural development.

### CROSS-REFERENCE TO RELATED APPLICATIONS

This patent application claims priority to U.S. Provisional Application No. 63/695,494, filed on Sep. 17, 2024, and U.S. Provisional Application No. 63/822,588, filed on Jun. 12, 2025, each of which is hereby incorporated by reference in its entirety.

### GOVERNMENT SUPPORT

This invention was made with government support under HD111079 awarded by the National Institutes of Health, and 2034835 awarded by the National Science Foundation. The government has certain rights in the invention.

### BACKGROUND

Protein undernutrition is a global risk factor for impaired growth and neurobehavioral development in children. Dysbiosis of the maternal gut microbiome, in response to environmental challenges such as infection, altered diet, and stress during pregnancy has also been increasingly associated with abnormalities in offspring brain function and behavior.

However, the critical periods, environmental interactions, and maternal versus neonatal influences on programming lasting behavioral abnormalities are poorly understood. The intestinal microbiota is an important modulator of brain function and behavior, but further research is necessary to resolve whether there are prenatal critical periods during which the maternal microbiome impacts the development of the fetal nervous system. Thus, methods of modifying the maternal microbiome, for example to compensate for a depleted maternal microbiome, prenatally (i.e., during gestation) are needed.

### SUMMARY

In some aspects, the present disclosure provides methods of promoting healthy, preventing impaired, or treating impaired neural development in a fetus, the methods comprising administering to a maternal subject gestating the fetus a composition comprising at least one metabolite selected from 3-indole sulfate, phenylacetylglycine, imidazole propionate, alpha-hydroxyisocaproate, 2-hydroxy-3-methylvalerate, alpha-hydroxyisovalerate, 1-methylhistamine, beta-hydroxisovalerate, 2R,3R-dihydroxybutyrate, and N-acetylleucine, or a combination thereof.

In some aspects, the present disclosure provides methods of promoting healthy, preventing impaired, or treating impaired neural development in a fetus, the methods comprising administering to a maternal subject gestating the fetus a composition comprising at least one short chain fatty acid selected from acetate, butyrate, and propionate, or a combination thereof.

In some embodiments, the maternal subject is undernourished. In some embodiments, the maternal subject is undernourished due to at least one of protein deficiency, poor weight gain, and fetal growth restriction.

In some embodiments, the subject is a mammal. In some embodiments, the subject is a human. In some embodiments, the method comprises administering the composition at least once during the first trimester of the maternal subject's gestation period. In some embodiments, the method comprises administering the composition at least once during a period that runs from the start of the third week after conception to the end of the eighth week after conception. In some embodiments, the method comprises administering the composition at least once during a period that runs from the 17^{th} day post conception (dpc) to the 52^{nd} dpc. In some embodiments, the method comprises administering the composition at least once during the second trimester of the maternal subject's gestation period. In some embodiments, the method comprises administering the composition at least once during the third trimester of the maternal subject's gestation period. In some embodiments, the fetus is an offspring of the maternal subject.

In some aspects, the present disclosure provides methods of conditioning a female subject for fostering healthy neural development in offspring, the methods comprising administering to the female subject a composition comprising at least one metabolite selected from 3-indole sulfate, phenylacetylglycine, imidazole propionate, alpha-hydroxyisocaproate, 2-hydroxy-3-methylvalerate, alpha-hydroxyisovalerate, 1-methylhistamine, beta-hydroxisovalerate, 2R,3R-dihydroxybutyrate, and N-acetylleucine, or a combination thereof; wherein the composition is administered at least once during a period that runs from the first day of an expected-but-missed menstruation to the end of gestation.

In some aspects, the present disclosure provides methods of conditioning a female subject for fostering healthy neural development in offspring, the methods comprising administering to the female subject a composition comprising at least one metabolite selected from short chain fatty acid selected from acetate, butyrate, and propionate, or a combination thereof; wherein the composition is administered at least once during a period that runs from the first day of an expected-but-missed menstruation to the end of gestation.

In some embodiments, the composition is administered at least once during a period that runs from the second day of the expected-but-missed menstruation to the end of gestation. In some embodiments, healthy neural development comprises a reduction in anxiety-like behavioral deficits in the offspring. In some embodiments, healthy neural development comprises prevention of learning and memory deficits in the offspring.

### BRIEF DESCRIPTION OF THE FIGURES

FIGS. 1A-1O show maternal protein restriction induces fetal growth restriction and maternal stress, and reduces postnatal survival, but does not influence early postnatal growth or maternal behavior. FIG. 1A: Macronutrient breakdown of control diet (CD) and protein restricted diet (PR) compared to standard lab chow. FIG. 1B: Fetal weight at E18.5 from specific pathogen free (SPF) CD and SPF PR (protein restriction) dams, all fetuses averaged within each litter (unpaired Welch's t-test, n=28, 21, from left to right). FIG. 1C: Maternal weight change, from E0.5 to E18.5, in SPF CD and SPF PR dams (unpaired Welch's t-test, n=26, 21, from left to right). FIG. 1D: Maternal diet eaten, from E0.5 to E18.5 in SPF CD and SPF PR dams (Mann Whitney test, n=26, 21, from left to right). FIG. 1E: Maternal spleen weight as a fraction of total E18.5 body weight post-transection (2-tailed Mann-Whitney test, n=25, 21). FIG. 1F: Left: E18.5 spleen cell frequencies of CD11B+ cells in SPF CD and PR dams (unpaired 2-tailed Welch's t-test per cell type, n=4). Right: E18.5 spleen cell frequencies of CD45+ cells in SPF CD and PR dams (unpaired 2-tailed Welch's t-test per cell type, n=4). FIG. 1G: E18.5 serum cytokines in SPF CD and PR dams (2-tailed Mann-Whitney test per cytokine, n=5, 4). Zeros=below the assay detection limit. FIG. 1H: Corticosterone measured in serum in SPF CD, SPF PR dams at E18.5 (unpaired Welch's t-test, n=10, 11, from left to right). FIG. 1I: Litter size at P0 (post-natal day 0), from SPF CD and SPF PR dams (Mann Whitney test, n=31, 50, from left to right). FIG. 1J: Litter size, pups per litters, measured at weaning, from cross-fostered groups SPF CD pups>CD dams, SPF CD pups>PR dams, SPF PR pups>CD dams, SPF PR pups>PR dams (two-way ANOVA with Sidak, n=6 litters per group). Top row refers to pup condition, bottom row refers to dam condition. FIG. 1K: Litter survival (percentage of total litters), from SPF CD pups>CD dams, SPF CD pups>PR dams, SPF PR pups>CD dams, SPF PR pups>PR dams (n=6, 15, 6, 34, from left to right). Top row refers to pup condition, bottom row refers to dam condition. FIG. 1L: Pup weights, all offspring averaged within each litter (two-way repeated measures ANOVA with Tukey, n=6 litters per group). FIG. 1M: Latency to right in pup righting reflex test, all offspring averaged within each litter (two-way repeated measures ANOVA with Tukey, n=6 litters per group). FIG. 1N, Latency to retrieve in maternal retrieval test (two-way repeated measures ANOVA with Tukey, n=6 dams per group). FIG. 1O, Left: Number of calls, ultrasonic vocalizations, 4 offspring averaged within each litter (two-way repeated measures ANOVA with Tukey, n=6 litters per group). Right: Mean duration of calls, ultrasonic vocalizations, 4 offspring averaged within each litter (two-way repeated measures ANOVA with Tukey, n=6 litters per group). Mean+/−SEM, *p<0.05, **p<0.01, ***p<0.001, ****p<0.0001, n.s. not significant.

FIGS. 2A-2H show the prenatal period of maternal protein restriction is critical for programming cognitive and anxiety-like behavioral deficits in adult offspring. FIG. 2A: Graphic of cross-fostering paradigm. FIG. 2B: Graphic of behavioral tests, open field test (left), Barnes maze (right). FIG. 2C: Time in center in open field test, all offspring averaged within each litter (two-way ANOVA with Sidak, n=6 litters per group). Top row refers to pup condition, bottom row refers to dam condition. FIG. 2D: Distance in center in open field test, controlled by total distance traveled, all offspring averaged within each litter (two-way ANOVA with Sidak, n=6 litters per group). Top row refers to pup condition, bottom row refers to dam condition. FIG. 2E: Left: Latency to escape in Barnes maze acquisition phase, all offspring averaged within each litter (n=6 litters per group). Right: AUC of latency to escape (two-way ANOVA with Sidak). Top row refers to pup condition, bottom row refers to dam condition. FIG. 2F: Left: Latency to target zone in Barnes maze acquisition phase, all offspring averaged within each litter (n=6 litters per group). Right: AUC of latency to target zone (two-way ANOVA with Sidak). Top row refers to pup condition, bottom row refers to dam condition. FIG. 2G: Time in target zone in Barnes maze probe trial, all offspring averaged within each litter (two-way ANOVA with Sidak, n=6 litters per group). FIG. 2H: Errors made in Barnes maze probe trial, all offspring averaged within each litter (two-way ANOVA with Sidak, n=6 litters per group). Mean+/−SEM, *p<0.05, **p<0.01, ***p<0.001, ****p<0.0001, n.s. not significant.

FIGS. 3A-3F show adult female offspring exhibit behavioral differences based on both gestational and rearing-associated protein restriction, whereas male offspring are based on gestational protein restriction alone. FIG. 3A: Left: Total distance traveled in open field test, all offspring averaged within each litter (two-way ANOVA with Sidak, n=6 litters per group). Right: Mean speed in open field test, all offspring averaged within each litter (two-way ANOVA with Sidak, n=6 litters per group). Top row refers to pup condition, bottom row refers to dam condition. FIG. 3B: Left: Time in center in open field test, male and female offspring (two-way ANOVA with Sidak for each sex, n=22, 20, 18, 18, 19, 25, 15, 14, from left to right). Right: Distance in center in open field test, controlled by total distance traveled, male and female offspring (two-way ANOVA with Sidak for each sex, n=22, 20, 18, 18, 19, 25, 15, 14, from left to right). FIG. 3C: Left: Latency to escape in Barnes maze acquisition phase, male offspring (n=22, 20, 19, 18). Middle: Latency to escape in Barnes maze acquisition phase, female offspring (n=19, 25, 15, 14). Right: AUC of latency to escape (two-way ANOVA with Sidak for each sex). FIG. 3D: Left: Latency to target zone in Barnes maze acquisition phase, male offspring (n=22, 20, 19, 18). Middle: Latency to target zone in Barnes maze acquisition phase, female offspring (n=19, 25, 15, 14). Right: AUC of latency to target zone (two-way ANOVA with Sidak for each sex). FIG. 3E: Time in target zone in Barnes maze probe trial, male and female offspring (two-way ANOVA with Sidak for each sex, n=22, 20, 19, 18, 19, 25, 15, 14, from left to right). FIG. 3F: Errors made in Barnes maze probe trial, male and female offspring (two-way ANOVA with Sidak for each sex, n=22, 20, 19, 18, 19, 25, 15, 14, from left to right). Mean+/−SEM, *p<0.05, **p<0.01, ***p<0.001, ****p<0.0001, n.s. not significant.

FIGS. 4A-4J show maternal protein restriction alters transcriptional and metabolomic profiles in the fetal brain. FIG. 4A: Heatmap of differentially expressed genes (DEGs) (505 up-regulated, 423 down-regulated; p<0.05) of E18.5 fetal brains from SPF CD or SPF PR dams with Euclidean clustering on rows (n=5 litters per group, 2 brains pooled per litter). FIG. 4B: Biological Process gene ontology (GO) of upregulated DEGs (p<0.05) and downregulated DEGs (p<0.05) of transcripts from E18.5 fetal brains from SPF CD or SPF PR dams (n=5 litters per group, 2 brains pooled per litter). FIG. 4C: Select STRING protein interaction analysis, highlighted based on relevance to neurodevelopmental phenotypes, of upregulated DEGs (p<0.05, red) and downregulated DEGs (p<0.05, blue) of transcripts from SPF PR vs. SPF CD fetal brain (n=5 litters per group, 2 brains pooled per litter). FIG. 4D: Volcano plot of transcripts from SPF PR vs. SPF CD fetal brain (differential expression analysis with DESeq2; n=5 litters per group, 2 brains pooled per litter). FIG. 4E: Heatmap of DEGs (p<0.05) from SPF PR vs. SPF CD fetal brain compared to the Simons Foundation SFARI database (n=5 litters per group, 2 brains pooled per litter). FIG. 4F: PCA of untargeted metabolomics from E18.5 fetal brains from SPF CD and SPF PR dams (n=6 litters per group, 1.5-2 brains pooled per litter). FIG. 4G: Metabolon network map showing positive and negative fold change. FIG. 4H: Top enriched KEGG pathways for significantly upregulated metabolites (p<0.05, left) and downregulated metabolites (p<0.05, right) for E18.5 fetal brains from SPF PR dams vs. SPF CD fetal brain (network analysis in MetaboAnalyst; bold pathways have q<0.05; teal symbols relate to analogous enriched pathways in SPF PR vs SPF CD maternal serum; *=enriched pathway in same direction; !=enriched pathway in opposite direction). FIG. 4I: Random forest analysis of top 30 metabolites differentiating SPF PR vs. SPF CD fetal brain with 100% predictive accuracy. FIG. 4J: Volcano plot of metabolites from SPF PR vs. SPF CD fetal brain (ANOVA contrasts from Metabolon; n=5 litters per group, 1.5-2 brains pooled per litter), highlighted based on relevance to neurodevelopmental phenotypes.

FIGS. 5A-5O show maternal protein restriction alters metabolomic profiles in maternal serum, and induces nutrient brain sparing in fetal brains at late gestation. FIG. 5A: Untargeted metabolomics PCA comparing serum from SPF CD and SPF PR dams (n=6 dams per group). FIG. 5B: Metabolon network map showing positive and negative fold change. FIG. 5C: Top enriched KEGG pathways for significantly upregulated metabolites (p<0.05, left) and downregulated metabolites (p<0.05, right) for serum from SPF PR dams compared to SPF CD dams (network analysis in MetaboAnalyst; bold pathways have q<0.05; teal symbols relate to analogous enriched pathways in SPF PR vs SPF CD fetal brain [see FIG. 4e]; *=enriched pathway in same direction; !=enriched pathway in opposite direction). FIG. 5D: Random forest analysis of top 30 metabolites differentiating SPF PR vs. SPF CD dam serum with 91.7% predictive accuracy. FIG. 5E: Volcano plot of SPF PR vs. SPF CD dam serum metabolites (ANOVA contrasts from Metabolon; n=6 dams per group), highlighted based on relevance to neurodevelopmental phenotypes. FIG. 5F-5O: Untargeted metabolomics of essential amino acids and glucose from E18.5 fetal brains from SPF CD and SPF PR dams (n=6 litters per group, 1.5-2 brains pooled per litter) and E18.5 serum from SPF CD and SPF PR dams (n=6 dams per group). Mean+/−SEM, *p<0.05, **p<0.01, ***p<0.001, ****p<0.0001, n.s. not significant.

FIGS. 6A-6N shows maternal protein restriction persistently alters maternal physiology in the postpartum period. FIG. 6A: Left: Shannon diversity index in postpartum dams (2-way repeated measures ANOVA with Sidak, pairwise by gestational diet, n=5, 4, 4, 5, 4, 4, 5, 4, 5, 5, 4, 5, 5, 5, 5, 5). Right: Pielou evenness index in postpartum dams (2-way repeated measures ANOVA with Sidak, pairwise by gestational diet, n=5, 4, 4, 5, 4, 4, 5, 4, 5, 5, 4, 5, 5, 5, 5, 5). FIG. 6B: PCA of weighted Unifrac in postpartum dams (QIIME2 PERMANOVA p=0.001, pairwise by gestational diet, n=5, 4, 4, 5, 4, 4, 5, 4, 5, 5, 4, 5, 5, 5, 5, 5). Axis 3=time pseudo-axis. FIG. 6C: Heatmap of significantly different taxa across gestation in postpartum dams based on gestational diet (CD>CD and PR>CD vs. PR>PR and CD>PR) (QIIME2 Kruskal Wallis test, adjusted p<0.05, n=5, 4, 4, 5, 4, 4, 5, 4, 5, 5, 4, 5, 5, 5, 5, 5). FIG. 6D: Left: Shannon diversity index in postnatal pups (2-way ANOVA with Dunnett per timepoint, PR>PR reference group, n=5, 5, 3, 5, 5, 5, 5, 4). Right: Pielou evenness index in postnatal pups (2-way ANOVA with Dunnett per timepoint, PR>PR reference group, n=5, 5, 3, 5, 5, 5, 5, 4) FIG. 6E: PCA of weighted UniFrac in postnatal pups (QIIME2 PERMANOVA p=0.001, pairwise by gestational diet, n=5, 5, 3, 5, 5, 5, 5, 4). Axis 3=time pseudo-axis. FIG. 6F: P11 maternal spleen weight fraction of total body weight (2-way ANOVA with Dunnett, PR>PR reference group, n=9, 6, 7, 14). FIG. 6G: Left: P11 maternal spleen cell frequencies of CD45+ cells (2-way ANOVA with Dunnett per cell type, PR>PR reference group, n=5, 6, 7, 9). Right: P11 maternal spleen cell frequencies of CD11B+ cells (2-way ANOVA with Dunnett per cell type, PR>PR reference group, n=5, 6, 7, 9). FIG. 6H: P11 maternal serum cytokines (2-way ANOVA with Dunnett per cytokine, PR>PR reference group, n=5, 6, 7, 9). Zeros<assay detection limit. FIG. 6I: P11 maternal serum corticosterone (2-way ANOVA with Dunnett, PR>PR reference group, n=5, 6, 7, 8). FIG. 6J: Left: P7 maternal milk proteins, lipids, lactose (2-way ANOVA with Dunnett per nutrient, PR>PR reference group, n=8, 6, 7, 11, 6, 5, 6, 9, 7, 5, 6, 10). Right: P7 maternal milk oligosaccharides (2-way ANOVA with Dunnett per nutrient, PR>PR reference group, n=12, 6, 6, 10). FIG. 6K: Left: P0 nesting score (2-tailed Mann-Whitney test, n=21, 54). Right: P1 nesting score (2-way ANOVA with Dunnett, PR>PR reference group, n=11, 12, 10, 34). FIG. 6L: Spearman correlation matrix of postpartum dam and pup timepoint-specific significant microbial metrics against other physiological metrics; displayed values are R². FIG. 6M: Left: Shannon diversity index in adult offspring (2-way ANOVA with Dunnett per sex, PR>PR reference group, n=7, 5, 6, 7, 6, 7, 6, 5). Right: Pielou evenness index in adult offspring (2-way ANOVA with Dunnett per sex, PR>PR reference group, n=7, 5, 6, 7, 6, 7, 6, 5). FIG. 6N: PCA of weighted UniFrac in adult offspring (QIIME2 PERMANOVA p=0.656, n=7, 5, 6, 7 males, 6, 7, 6, 5 females). Cones=female, spheres=male. Error bars are mean+/−SEM.

FIGS. 7A-7C show murine maternal protein restriction or human fetal growth restriction reduces diversity of the gut microbiome. FIG. 7A: Left: Shannon diversity index across gestation in SPF CD and SPF PR dams (two-way repeated measures ANOVA with Sidak, n=5 dams per group). Right: Pielou evenness index across gestation in SPF CD and SPF PR dams (two-way repeated measures ANOVA with Sidak, n=5 dams per group). FIG. 7B: PCA of weighted UniFrac across gestation in SPF CD and SPF PR dams (n=5 dams per group). Axis 3=time pseudo-axis. FIG. 7C: Heatmap of significantly different taxa across gestation in SPF CD and SPF PR dams (Qiime2 Kruskal Wallis test, q<0.05, n=5 dams per group). Error bars are mean+/−SEM.

FIGS. 8A-F shows that murine maternal protein restriction or human fetal growth restriction reduces diversity of the gut microbiome. FIG. 8A: Left: Shannon diversity index in the first 6 weeks of life in control (Con) and fetal growth restriction (FGR) infants (unpaired Welch's t-test per timepoint, n=37 (with 1-4 timepoints each), 16 (with 1-5 timepoints each) infants from top to bottom). Right: Pielou evenness index in the first 6 weeks of life in Con and FGR infants (unpaired Welch's t-test per timepoint, n=37 (with 1-4 timepoints each), 16 (with 1-5 timepoints each) infants from top to bottom). FIG. 8B: PCA of weighted unifrac of Con and FGR infants across the first six weeks of life (n=34 Con with 1-4 timepoints each, 16 intrauterine growth restriction (IUGR) with 1-5 timepoints each). FIG. 8C: Taxa bar plots of taxa>1% total reads in Con or FGR infants in week 2 of life (n=26, 10, infants from left to right, ** survived correction by Kruskal Wallis test in Qiime2). FIG. 8D: Spearman correlation matrix of Shannon diversity index at week 3 and Staphylococcus genus % reads at week 2, against clinical metadata (Con n=16, 22; FGR n=9, 10, from left to right). Displayed values are R2. FIG. 8E: Left: Bayley Cognitive assessment scores across the first two years of life in Con and FGR infants (2-way ANOVA Sidak, n=28 Con with 1-3 visits each, 15 IUGR with 1-3 visits each). Middle: Bayley Motor assessment scores across the first two years of life in Con and FGR infants (2-way ANOVA Sidak, n=28 Con with 1-3 visits each, 15 IUGR with 1-3 visits each). Right: Bayley Language assessment scores across the first two years of life in Con and FGR infants (2-way ANOVA Sidak, n=28 Con with 1-3 visits each, 15 IUGR with 1-3 visits each). FIG. 8F: Spearman correlation matrix of Shannon diversity index at week 3 and Staphylococcus genus % reads at week 2 against Bayley cognitive, motor, and language composite scores from 6 months (Con n=10, FGR=7; Con n=10, FGR=8, from left to right), 12 months (Con n=8, FGR=3; Con n=7, FGR=2, from left to right), and 18-24 months (Con n=8, FGR=4; Con n=7, FGR=4, from left to right). Displayed values are R2. Mean+/−SEM, *p<0.05, **p<0.01, ***p<0.001, ****p<0.0001, n.s. not significant.

FIGS. 9A-9K shows depletion of the maternal gut microbiome induces distinct and additive fetal brain transcriptomic and metabolomic responses to maternal protein restriction. FIG. 9A: Timeline of antibiotics (ABX) treatment prior to and throughout gestation. FIG. 9B: Venn diagram of differentially expressed genes (DEGs) (p<0.05) in the fetal brains from SPF PR compared to SPF CD dams and in the fetal brains from ABX PR compared to SPF PR dams (n=5 litters per group, 2 brains pooled per litter). The second row shows upregulated genes. The third row shows downregulated genes. The center of the Venn shows the overlapping genes from both comparisons. Of these 86 genes upregulated in SPF PR vs SPF CD, 3 were similarly upregulated in ABX PR vs SPF PR and 83 were instead downregulated. Of the 74 genes downregulated in SPF PR vs SPF CD, 3 were similarly downregulated in ABX PR vs SPF PR while 71 were instead upregulated. Red=up-regulated, blue=down-regulated. For overlapping genes from both comparisons (venn center), red=up-regulated, blue=down-regulated in SPF PR vs. SPF CD; colored numbers in parentheses match directionality in ABX PR vs. SPF PR. FIG. 9C: Biological Process gene ontology (GO) of upregulated DEGs (p<0.05) and downregulated DEGs (p<0.05) of fetal brain transcripts from ABX PR dams and SPF PR dams (n=5 litters per group, 2 brains pooled per litter). FIG. 9D: Select STRING protein interaction analysis, highlighted based on relevance to neurodevelopmental phenotypes, of upregulated DEGs (p<0.05, red) and downregulated DEGs (p<0.05, blue) of ABX PR vs SPF PR fetal brain transcripts (n=5 litters, 2 brains pooled per litter). FIG. 9E: Volcano plot of transcripts from ABX PR vs. SPF PR fetal brain (differential expression analysis with DESeq2; n=5 litters, 2 brains pooled per litter). FIG. 9F: Left: Venn diagram of DEGs (p<0.05) from ABX PR vs. SPF PR fetal brain compared to the Simons Foundation SFARI database. Right: fold change of 5 overlapping genes from venn center (SPF PR vs. SPF CD, ABX PR vs. SPF PR; differential expression analysis with DESeq2; n=5 litters, 2 brains pooled per litter). FIG. 9G: Venn diagram of differential metabolites (p<0.05) in the fetal brains from SPF PR compared to SPF CD dams and in the fetal brains from ABX PR compared to SPF PR dams (n=5 litters per group, 1.5-2 brains pooled per litter). The second row shows upregulated genes. The third row shows downregulated genes. For the overlapping genes from both comparisons (center of the Venn), 15 genes were up-regulated and 7 genes were down-regulated in SPF PR vs. SPF CD; the numbers in the parentheses show that for the 15 upregulated genes, 8 were increased and 7 were decreased in ABX PR vs SPF PR and for the downregulated genes, 23 were increased and 2 were decreased in ABX PR vs SPF PR. FIG. 9H: Metabolon network map showing positive and negative fold change. FIG. 9F: Top enriched KEGG pathways for significantly up-regulated metabolites (p<0.05, left) and down-regulated metabolites (p<0.05, right) from fetal brains of ABX PR dams compared to SPF PR dams (bold pathways have q<0.05; teal symbols relate to analogous enriched pathways in SPF PR vs SPF CD fetal brain [see FIG. 5c]; *=enriched pathway in same direction; !=enriched pathway in opposite direction). FIG. 9J: Random forest analysis of top 30 metabolites differentiating ABX PR vs. SPF PR fetal brain with 93.3% predictive accuracy. FIG. 9K: Volcano plot of metabolites from ABX PR vs. SPF PR fetal brain (ANOVA contrasts from Metabolon; n=5 litters, 1.5-2 brains pooled per litter). Error bars are mean+/−SEM.

FIGS. 10A-10H show maternal microbiome-informed interventions differentially modify risk for neurobehavioral deficits induced by maternal protein restriction during pregnancy. FIG. 10A: Graphic of cross-fostering paradigm for ABX experiments. FIG. 10B: Left: Latency to target zone in Barnes maze, all offspring averaged within each litter (n=5 litters per group). Dotted line indicates average value for SPF CD litters. Right: AUC of latency to target zone (unpaired Welch's t-test). Top row refers to pup condition, bottom row refers to dam condition. FIG. 10C: Heatmap of metabolites chosen for 10M supplementation, hierarchical clustering around 0, SD=1 (n=6 litters for each group/tissue type, 2 fetal brains or 1 dam serum pooled per litter). FIG. 10D: Graphic of cross-fostering paradigm for 10M experiments. FIG. 10E: Time in center in open field test, male and female offspring (Mann Whitney test for each sex, n=13, 14, 9, 19, from left to right). FIG. 10F: Distance in center in open field test, controlled by total distance traveled, male and female offspring (Mann Whitney test for each sex, n=13, 14, 9, 19, from left to right). FIG. 10G: Time in target zone in Barnes maze probe trial, male and female offspring (unpaired Welch's t-test for each sex, n=13, 14, 8, 18, from left to right). FIG. 10H: Errors in Barnes maze probe trial, male and female offspring (Mann-Whitney test for each sex, n=13, 14, 8, 18, from left to right). Mean+/−SEM, *p<0.05, **p<0.01, ***p<0.001, ****p<0.0001, n.s. not significant.

FIGS. 11A-11H show maternal microbial depletion moderately impacts gross measures of maternal and offspring health. FIG. 11A: Fetal weight at E18.5 from SPF CD, SPF PR, ABX CD, ABX PR dams, all fetuses averaged within each litter (two-way ANOVA with Sidak, n=28, 21, 16, 17, from left to right). SPF data same as in FIG. 1B. FIG. 11B: Maternal weight change, from E0.5 to E18.5 post-transection, in SPF CD, SPF PR, ABX CD, ABX PR dams (two-way ANOVA with Sidak, n=26, 21, 16, 17, from left to right). SPF data same as in FIG. 1C. FIG. 11C: Diet eaten, from E0.5 to E18.5, in SPF CD, SPF PR, ABX CD, ABX PR dams (two-way ANOVA with Sidak, n=26, 21, 16, 17, from left to right). SPF data same as in FIG. 1D. FIG. 11D: Corticosterone measured in serum in SPF CD, SPF PR, ABX CD, ABX PR dams at E18.5 (two-way ANOVA with Sidak, n=10, 11, 10, 11, from left to right). SPF data same as in FIG. 1E. FIG. 11E: Litter size, pups per litters, measured at P0, from SPF PR and ABX PR dams (Mann-Whitney test, n=21, 14, from left to right). Dotted line indicates average value for SPF CD litters. FIG. 11F: Litter survival (percentage of total litters), from SPF PR pups>SPF PR dams and ABX PR pups>SPF PR dams (n=13, 10, from left to right). Dotted line indicates average value for SPF CD litters. Top row refers to pup condition, bottom row refers to dam condition. FIG. 11G: Pup weights, all offspring averaged within each litter (two-way mixed effects analysis with Sidak, n=7 litters per group). Dotted line indicates average value for SPF CD litters. FIG. 11H: Adult weights, male and female offspring (two-way ANOVA with Sidak, n=24, 20, 23, 23, from left to right). Mean+/−SEM, *p<0.05, **p<0.01, ***p<0.001, ****p<0.0001, n.s. not significant.

FIGS. 12A-12I shows adult offspring do not exhibit sexually dimorphic behavioral responses to gestational protein restriction and maternal microbiome depletion. FIG. 12A: Left: Time in center in open field test, all offspring averaged within each litter (Mann Whitney test, n=7 litters per group). Top row refers to pup condition, bottom row refers to dam condition. Dotted line indicates average value for SPF CD litters. Left-middle: Distance in center in open field test, controlled by total distance traveled, all offspring averaged within each litter (unpaired Welch's t-test, n=7 litters per group). Top row refers to pup condition, bottom row refers to dam condition. Dotted line indicates average value for SPF CD litters. Right-middle: Total distance traveled in open field test, all offspring averaged within each litter (Mann Whitney test, n=7 litters per group). Top row refers to pup condition, bottom row refers to dam condition. Dotted line indicates average value for SPF CD litters. Right: Mean speed in open field test, all offspring averaged within each litter (Mann Whitney test, n=7 litters per group). Top row refers to pup condition, bottom row refers to dam condition. Dotted line indicates average value for SPF CD litters. FIG. 12B: Left: Latency to escape in Barnes maze, all offspring averaged within each litter (n=5 litters per group). Dotted line indicates average value for SPF CD litters. Right: AUC of latency to escape (unpaired Welch's t-test). Top row refers to pup condition, bottom row refers to dam condition. FIG. 12C: Time in target zone in Barnes maze probe phase, all offspring averaged within each litter (unpaired Welch's t-test, n=5 litters per group). Dotted line indicates average values for SPF CD litters. FIG. 12D: Errors made in Barnes maze probe phase, all offspring averaged within each litter (unpaired Welch's t-test, n=5 litters per group). Dotted line indicates average values for SPF CD litters. Mean+/−SEM, *p<0.05, **p<0.01, ***p<0.001, ****p<0.0001, n.s. not significant. FIG. 12E: Left: Time in center in open field test, male and female offspring (Mann Whitney test for each sex, n=24, 20, 23, 23, from left to right). Right: Distance in center in open field test, controlled by total distance traveled, male and female offspring (Mann Whitney test for each sex, n=24, 20, 23, 23, from left to right). FIG. 12F: Left: Latency to escape in Barnes maze acquisition phase, male offspring (n=17, 16). Middle: Latency to escape in Barnes maze acquisition phase, female offspring (n=17, 16). Right: AUC of latency to escape (unpaired Welch's t-test for each sex). FIG. 12G: Left: Latency to target zone in Barnes maze acquisition phase, male offspring (n=17, 16). Middle: Latency to target zone in Barnes maze acquisition phase, female offspring (n=17, 16). Right: AUC of latency to target zone (unpaired Welch's t-test for each sex). FIG. 12H: Time in target zone in Barnes maze probe trial, male and female offspring (Mann-Whitney test for each sex, n=17, 17, 16, 16, from left to right). FIG. 12I: Errors made in Barnes maze probe trial, male and female offspring (Mann-Whitney test for each sex, n=17, 17, 16, 16, from left to right). Mean+/−SEM, *p<0.05, **p<0.01, ***p<0.001, ****p<0.0001, n.s. not significant.

FIGS. 13A-13Q show maternal microbial SCFA supplementation has limited impact on gross measures of maternal and offspring health. FIG. 13A: Graphic of gestational supplementation and cross-fostering paradigm for SCFA experiments. FIG. 13B: Fetal weight at E18.5 from SPF PR+Na and SPF PR+SCFA dams, all fetuses averaged within each litter (unpaired Welch's t-test, n=9, 11, from left to right). Dotted line indicates average value for SPF CD fetuses. FIG. 13C: Maternal weight change, from E0.5 to E18.5 post-transection, in SPF PR+Na and SPF PR+SCFA dams (unpaired Welch's t-test, n=9, 11, from left to right). Dotted line indicates average value for SPF CD dams. FIG. 13D: Diet eaten, from E0.5 to E18.5 in SPF PR+Na and SPF PR+SCFA dams (unpaired Welch's t-test, n=9, 11, from left to right). Dotted line indicates average value for SPF CD dams. FIG. 13E: Corticosterone measured in serum in SPF PR+Na and SPF PR+SCFA dams at E18.5 (unpaired Welch's t-test, n=9, 11, from left to right). Dotted line indicates average value for SPF CD dams. FIG. 13F: Litter size, pups per litters, measured at P0, from SPF PR+Na and SPF PR+SCFA dams (unpaired Welch's t-test, n=17, 10, from left to right). Dotted line indicates average value for SPF CD litters. FIG. 13G: Litter survival (percentage of total litters), from SPF PR+Na pups>SPF PR+Na dams and SPF PR+SCFA pups>SPF PR+SCFA dams (n=17, 9, from left to right). Dotted line indicates average value for SPF CD litters. Top row refers to pup condition, bottom row refers to dam condition. FIG. 13H: Pup weights, all offspring averaged within each litter (two-way repeated measures ANOVA with Sidak, n=9, 6 litters per group, from top to bottom). Dotted line indicates average value for SPF CD litters. FIG. 13I: Adult weights, male and female offspring (two-way ANOVA with Sidak, n=21, 14, 18, 16, from left to right). FIG. 13J: Fetal weight at E18.5 from SPF PR+Veh and SPF PR+10M dams, all fetuses averaged within each litter (unpaired Welch's t-test, n=11, 10, from left to right). Dotted line indicates average value for SPF CD fetuses. FIG. 13K: Maternal weight change, from E0.5 to E18.5 post-transection, in SPF PR+Veh and SPF PR+10M dams (Mann-Whitney test, n=11, 10, from left to right). Dotted line indicates average value for SPF CD dams. FIG. 13L: Diet eaten, from E0.5 to E18.5 in SPF PR+Veh and SPF PR+10M dams (Mann-Whitney test, n=11, 10, from left to right). Dotted line indicates average value for SPF CD dams. FIG. 13M: Corticosterone measured in serum in SPF PR+Veh and SPF PR+10M dams at E18.5 (unpaired Welch's t-test, n=11, 10, from left to right). Dotted line indicates average value for SPF CD dams. FIG. 13N: Litter size, pups per litters, measured at P0, from SPF PR+Veh and SPF PR+10M dams (Mann-Whitney test, n=15, 8, from left to right). Dotted line indicates average value for SPF CD litters. FIG. 13O, Litter survival (percentage of total litters), from SPF PR+Veh pups>SPF PR+Veh dams and SPF PR+10M pups>SPF PR+10M dams (n=15, 8, from left to right). Dotted line indicates average value for SPF CD litters. Top row refers to pup condition, bottom row refers to dam condition. FIG. 13P: Pup weights, all offspring averaged within each litter (two-way repeated measures mixed effects analysis with Sidak, n=4, 6 litters per group, from top to bottom). Dotted line indicates average value for SPF CD litters. FIG. 13Q: Adult weights, male and female offspring (two-way ANOVA with Sidak, n=13, 14, 8, 19, from left to right). Mean+/−SEM, *p<0.05, **p<0.01, ***p<0.001, ****p<0.0001, n.s. not significant.

FIGS. 14A-14J show maternal microbial SCFA supplementation does not influence anxiety-like, locomotor, or cognitive behavioral measures in adult offspring exposed to gestational protein restriction. FIG. 14A: Left: Time in center in open field test, all offspring averaged within each litter (Mann Whitney test, n=6 litters per group). Top row refers to pup condition, bottom row refers to dam condition. Dotted line indicates average value for SPF CD litters. Left-middle: Distance in center in open field test, controlled by total distance traveled, all offspring averaged within each litter (unpaired Welch's t-test, n=6 litters per group). Top row refers to pup condition, bottom row refers to dam condition. Dotted line indicates average value for SPF CD litters. Right-middle: Total distance traveled in open field test, all offspring averaged within each litter (unpaired Welch's t-test, n=6 litters per group). Top row refers to pup condition, bottom row refers to dam condition. Dotted line indicates average value for SPF CD litters. Right: Mean speed in open field test, all offspring averaged within each litter (unpaired Welch's t-test, n=6 litters per group). Top row refers to pup condition, bottom row refers to dam condition. Dotted line indicates average value for SPF CD litters. FIG. 14B: Left: Latency to escape in Barnes maze, all offspring averaged within each litter (n=6 litters per group). Dotted line indicates average value for SPF CD litters. Right: AUC of latency to escape (unpaired Welch's t-test). Top row refers to pup condition, bottom row refers to dam condition. FIG. 14C: Left: Latency to target zone in Barnes maze, all offspring averaged within each litter (n=6 litters per group). Dotted line indicates average value for SPF CD litters. Right: AUC of latency to target zone (unpaired Welch's t-test). Top row refers to pup condition, bottom row refers to dam condition. FIG. 14D: Time in target zone in Barnes maze probe trial, all offspring averaged within each litter (unpaired Welch's t-test, n=6 litters per group). Dotted line indicates average values for SPF CD litters. FIG. 14E: Errors made in Barnes maze probe trial, all offspring averaged within each litter (unpaired Welch's t-test, n=6 litters per group). Dotted line indicates average values for SPF CD litters. FIG. 14F: Left: Time in center in open field test, male and female offspring (Mann Whitney test for each sex, n=20, 14, 18, 16, from left to right). Right: Distance in center in open field test, controlled by total distance traveled, male and female offspring (unpaired Welch's t-test for each sex, n=20, 14, 18, 16, from left to right). FIG. 14G: Left: Latency to escape in Barnes maze acquisition phase, male offspring (n=21, 13). Middle: Latency to escape in Barnes maze acquisition phase, female offspring (n=18, 16). Right: AUC of latency to escape (unpaired Welch's t-test for each sex). FIG. 14H: Left: Latency to target zone in Barnes maze acquisition phase, male offspring (n=21, 13). Middle: Latency to target zone in Barnes maze acquisition phase, female offspring (n=18, 16). Right: AUC of latency to target zone (unpaired Welch's t-test for each sex). FIG. 14I: Time in target zone in Barnes maze probe trial, male and female offspring (Mann-Whitney test for each sex, n=21, 13, 18, 16, from left to right). FIG. 14J: Errors made in Barnes maze probe trial, male and female offspring (Mann-Whitney test for each sex, n=21, 13, 18, 16, from left to right). Mean+/−SEM, *p<0.05, **p<0.01, ***p<0.001, ****p<0.0001, n.s. not significant.

FIG. 15A-15G show maternal 10M supplementation does not influence anxiety-like, locomotor, or cognitive behavioral measures in adult offspring exposed to gestational protein restriction. FIG. 15A: Left: Time in center in open field test, all offspring averaged within each litter (unpaired Welch's t-test, n=4, 6 litters per group, from left to right). Top row refers to pup condition, bottom row refers to dam condition. Dotted line indicates average value for SPF CD litters. Left-middle: Distance in center in open field test, controlled by total distance traveled, all offspring averaged within each litter (unpaired Welch's t-test, n=4, 6 litters per group, from left to right). Top row refers to pup condition, bottom row refers to dam condition. Dotted line indicates average value for SPF CD litters. Right-middle: Total distance traveled in open field test, all offspring averaged within each litter (unpaired Welch's t-test, n=4, 6 litters per group, from left to right). Top row refers to pup condition, bottom row refers to dam condition. Dotted line indicates average value for SPF CD litters. Right: Mean speed in open field test, all offspring averaged within each litter (unpaired Welch's t-test, n=4, 6 litters per group, from left to right). Top row refers to pup condition, bottom row refers to dam condition. Dotted line indicates average value for SPF CD litters. FIG. 15B: Left: Latency to escape in Barnes maze, all offspring averaged within each litter (n=4, 6 litters per group, from top to bottom). Dotted line indicates average value for SPF CD litters. Right: AUC of latency to escape (unpaired Welch's t-test). Top row refers to pup condition, bottom row refers to dam condition. FIG. 15C: Left: Latency to target zone in Barnes maze, all offspring averaged within each litter (n=4, 6 litters per group, from top to bottom). Dotted line indicates average value for SPF CD litters. Right: AUC of latency to target zone (unpaired Welch's t-test). Top row refers to pup condition, bottom row refers to dam condition. FIG. 15D: Time in target zone in Barnes maze probe trial, all offspring averaged within each litter (unpaired Welch's t-test, n=4, 6 litters per group, from left to right). Dotted line indicates average values for SPF CD litters. FIG. 15E: Errors made in Barnes maze probe trial, all offspring averaged within each litter (unpaired Welch's t-test, n=4, 6 litters per group, from left to right). Dotted line indicates average values for SPF CD litters. FIG. 15F: Left: Latency to escape in Barnes maze acquisition phase, male offspring (n=13, 14). Middle: Latency to escape in Barnes maze acquisition phase, female offspring (n=9, 18). Right: AUC of latency to escape (unpaired Welch's t-test for each sex). FIG. 15G: Left: Latency to target zone in Barnes maze acquisition phase, male offspring (n=13, 14). Middle: Latency to target zone in Barnes maze acquisition phase, female offspring (n=9, 18). Right: AUC of latency to target zone (unpaired Welch's t-test for each sex). Mean+/−SEM, *p<0.05, **p<0.01, ***p<0.001, ****p<0.0001, n.s. not significant.

### DETAILED DESCRIPTION

### Definitions

“Impaired neural development,” as used herein, refers to abnormalities in brain function and behavior, in offspring. Examples of impaired neural development include, but are not limited to, increased anxiety, impaired learning, and impaired memory (e.g., poor performance on cognitive or spatial tests). Examples of “healthy neural development,” as used herein, include, but are not limited to, healthy development in fetal brain gene expression, fetal axonogenesis, fetal axon development, and/or adult or pediatric tactile sensory behavior.

“Microbiome,” as used herein, refers to the microorganisms in a given environment, such as the body or a part of the body. The “maternal microbiome,” as used herein, refers to the microorganisms in a maternal subject (i.e., a pregnant or gestating subject), particularly in the gut of the maternal subject. The gut microbiome modulates the bioavailability of hundreds of biochemicals in the circulating blood. During pregnancy, the maternal gut environment supplies nutrients and growth factors, from the maternal diet and other nutritional intake, to nurture offspring growth.

“Metabolite,” as used herein, includes, but is not limited to, an intermediate or end product of metabolism.

“Protein Restriction,” as used herein, or “protein undernutrition,” refers to consumption of decreased protein compared to a standard diet or physician recommended diet. This includes, but is not limited to, an individual who is experiencing the symptoms of protein undernutrition, such as fatigue or loss of muscle mass. An individual with protein undernutrition also includes an individual who is not experiencing symptoms but is nevertheless consuming less than a recommended amount of protein.

A “depleted” maternal microbiome is characterized by a reduced level of one or more microbial species (e.g., one or more bacterial species), such as 95%, 90%, 85%, 80%, 75%, 70%, 65%, 60%, 55%, 50%, 45%, 40%, 35%, 30%, 25%, 20%, 15%, 10%, 5%, 4%, 3%, 2%, 1%, 0.5%, or 0.1% of the level relative to a maternal subject without a depleted maternal microbiome.

“Antibiotic-treated” (ABX) subjects, as used herein, are subjects treated with one or more antibiotic compounds, many representative examples of which are known in the art.

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); and/or mammals, including commercially relevant mammals such as cattle, pigs, horses, sheep, goats, cats, and/or dogs. Preferred subjects are humans.

An “ovulating” female subject, as used herein, refers to a female subject having a regular cycle of menses, e.g., a female between menarche and menopause that is not employing hormonal birth control that inhibits ovulation. A “fertile” female subject, as used herein, refers to an ovulating female subject able to conceive offspring.

As used herein, a therapeutic that “prevents” a disorder or condition refers to a compound or composition that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” includes prophylactic and/or therapeutic treatments. The term “prophylactic or therapeutic” treatment is art-recognized and includes administration to the subject of one or more of the disclosed compositions. If it is administered prior to clinical manifestation of the unwanted condition (e.g., disease or other unwanted state of the subject) then the treatment is prophylactic (i.e., it protects the subject against developing the unwanted condition), whereas if it is administered after manifestation of the unwanted condition, the treatment is therapeutic (i.e., it is intended to diminish, ameliorate, or stabilize the existing unwanted condition or side effects thereof).

As used herein, the term “about” is defined as being close to as understood by one of ordinary skill in the art. In one non-limiting embodiment, the term “about” is defined to be within 10%, preferably within 5%, more preferably within 1%, and most preferably within 0.5%.

As used herein, “stably stored” or “storage-stable” refer to a composition in which cells are able to withstand storage for extended periods of time (e.g., at least one month, or two, three, four, six, or twelve months or more) with a less than 95%, less than 90%, less than 80%, less than 70%, less than 60%, less than 50%, less than 40%, less than 30%, less than 20%, less than 15%, less than 10%, less than 5%, or less than 1% decrease in cell viability.

As used herein, the phrase “conjoint administration” refers to any form of administration of two or more different therapeutic compounds such that the second compound is administered while the previously administered therapeutic compound is still effective in the body (e.g., the two compounds are simultaneously effective in the subject, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, a subject who receives such treatment can benefit from a combined effect of different therapeutic compounds.

As used herein, the terms “improve”, “increase”, “inhibit”, “decrease” and “reduce”, and grammatical equivalents thereof, indicate qualitative or quantitative difference from a reference.

As used herein, “level” is with respect to a molecule such as a nucleic acid or polypeptide, “level” is used to refer to a measure indicative of an amount, concentration, ratio, or activity of the molecule, e.g., in a particular context such as a tissue, sample, organism, or a context representative thereof. An amount can be, for example, a mass or number of molecules. A concentration can be an amount relative to a context value, e.g., per a unit of mass or volume. A ratio can be a relationship between two values, such as an experimental value and a reference control value. Activity can be a measure of a function associated with a molecule, and can in various instances be measured relative to a context value, e.g., per a unit of mass or volume. Those of skill in the art will appreciate that the metric by which a level is expressed can vary depending, e.g., on the assay and purpose. Those of skill in the art will further appreciate that metrics such as amount, concentration, ratio, and activity are often interrelated and/or qualitatively or quantitatively informative of each other.

Unless otherwise defined, all technical and scientific terms used herein have the same meaning as commonly understood by one of ordinary skill in the art to which this disclosure pertains. Preferred methods and materials are described below, although methods and materials similar or equivalent to those described herein can also be used in the practice or testing of the presently disclosed methods and compositions.

### Maternal Protein Restriction (PR)

Protein undernutrition is a global risk factor for impaired growth and neurobehavioral development in children. In mouse models of fetal growth restriction, limiting maternal protein intake particularly during pregnancy leads to cognitive and anxiety-like behavioral abnormalities in adult offspring, indicating a critical role for the gestational period. By cross-fostering newborn mice to dams previously exposed to either low protein or standard diet, the present disclosure demonstrates that the adult behavioral impairments require diet-induced conditioning of both fetal development and maternal peripartum physiology, rather than either alone. This suggests that protein undernutrition during pregnancy directly disrupts fetal neurodevelopment and indirectly alters maternal state in ways that interact postnatally to precipitate behavioral deficits. Consistent with this, maternal protein restriction during pregnancy reduces the diversity of the maternal gut microbiome, modulates maternal serum metabolomic profiles, and yields widespread alterations in fetal brain transcriptomic and metabolomic profiles, including subsets of microbiome-dependent metabolites. Depletion of the maternal microbiome in protein-restricted dams further alters fetal brain gene expression and exacerbates neurocognitive behavior in adult offspring, suggesting that the maternal microbiome modifies the impact of gestational protein undernutrition on risk for neurobehavioral impairment in the offspring.

Protein undernutrition is a global risk factor for childhood stunting, which is co-morbid with lasting neurological disabilities, including cognitive impairment and anxiety. In humans and animal models, abnormalities in the maturation and function of the gut microbiome contribute to malnutrition-induced stunting, but standard therapeutic foods have limited effectiveness in supporting persistent microbial rehabilitation. There is increasing evidence that bacterial treatments and custom microbiota-directed diets ameliorate growth restriction in animal models of malnutrition and in malnourished children. This raises the prospect of using microbiome-based treatments to combat malnutrition-induced growth defects. However, current medical and nutritional interventions that treat childhood stunting are often inadequate to ameliorate co-morbid neurobehavioral impairments. Individuals who experienced protein-energy malnutrition during the first year of life displayed cognitive impairment and depressive symptoms during adolescence and adulthood, despite adequate nutritional rehabilitation and growth recovery during childhood. Similarly, supplementing stunted infants with a milk-based formula failed to ameliorate heightened anxiety and cognitive impairments in adolescence. Whether alterations in the microbiome contribute to the neurological comorbidities caused by protein undernutrition, and whether microbiome-based interventions can be used to ameliorate them, is poorly understood.

The maternal microbiome is demonstrated herein to be a modifier of adverse neurological outcomes in offspring of protein undernourished dams. Maternal protein restriction reduces diversity of the maternal microbiome and elicits widespread alterations in maternal-fetal metabolomic profiles and fetal brain gene expression, including subsets of maternal microbiome-dependent metabolites and genes. Further depleting the maternal microbiome of protein-restricted dams substantially alters transcriptomic and metabolomic profiles in fetal brains, where only a small fraction of metabolites and genes differentially regulated by maternal microbiome depletion overlap with those altered by protein restriction alone. Furthermore, depleting the maternal microbiome exacerbates cognitive, but not anxiety-like, behavioral impairments observed in adult offspring of protein-restricted dams. These results suggest that wholesale depletion of the maternal microbiome elicits select brain and behavioral changes in offspring of protein-restricted dams through mechanisms that are largely independent of those caused by maternal protein restriction alone. As such, reductions in microbial diversity likely modify, but do not directly mediate, adverse effects of maternal protein restriction on offspring neurodevelopment. In addition, human preterm infants with FGR display early postnatal decreases in microbial diversity which correlate with reduced infant cognitive scores compared to non-FGR counterparts.

### Methods of Treatment and Prevention

Supplementing protein-restricted dams with a cocktail of ten diet- and microbiome-dependent metabolites during pregnancy prevents anxiety-like behavior and cognitive impairment in their adult offspring. The metabolites were selected based on their significant decreases in both fetal brain and maternal serum of protein-restricted dams compared to standard protein-fed controls, and their further modulation by depletion of the maternal microbiome. Five of the ten metabolites (alpha-hydroxyisocaproate, 2-hydroxy-3-methylvalerate, alpha-hydroxyisovalerate, beta-hydroxyisovalerate, and N-acetylleucine) are dietary catabolites of the branched chain amino acids (BCAAs) leucine, isoleucine, and valine. The gut microbiota is capable of both producing and breaking down BCAAs. BCAAs are also implicated in neural development and function following insult: alpha-hydroxyisocaproate, 2-hydroxy-3-methylvalerate, alpha-hydroxyisovalerate, and N-acetylleucine were increased in fetal brain following exposure to intrauterine inflammation, and N-acetylleucine reduced cortical inflammation and apoptosis and increased memory in novel object recognition task after traumatic brain injury. In addition to these five BCAA-related metabolites, 3-indoxyl sulfate, imidazole propionate, and phenylacetylglycine are well established metabolites of the gut microbiome produced by sulfonation of bacterially-derived indole, direct bacterial synthesis from histidine, and bacterial conversion of phenylalanine, respectively. They have also been implicated in neurodevelopment: imidazole propionate promoted thalamocortical axonogenesis in fetal brains from offspring of antibiotic-depleted dams, and all three have been reported to vary across postnatal development in mouse forebrain. The final 10M metabolites, 1-methylhistamine and 2R,3R-dihydroxybutyrate (commonly referred to as 4-deoxyerythronic acid), have only correlational ties to the gut microbiome and brain development. The former, a major metabolite of histamine, has been correlated with microbes, primarily of the order Clostridiales and genus Lactobacillus, while the latter, a metabolite of L-threonine, was downregulated in the plasma of MDD patients after escitalopram treatment. Specific microbial metabolites are increasingly linked to neurodevelopment and behavior.

To explore the potential for microbiome-targeted interventions, maternal treatment with short chain fatty acids (acetate, propionate, and butyrate) or a cocktail of 10 diet- and microbiome-dependent metabolites (3-indole sulfate, phenylacetylglycine, imidazole propionate, alpha-hydroxyisocaproate, 2-hydroxy-3-methylvalerate, alpha-hydroxyisovalerate, 1-methylhistamine, beta-hydroxisovalerate, 2R,3R-dihydroxybutyrate, and N-acetylleucine) was found to promote healthy fetal development and postnatal behavior. This highlights the impact of prenatal maternal protein undernutrition on fetal neurodevelopment and adverse neurobehavioral trajectories in offspring, which are mitigated by microbiome-targeted interventions during pregnancy.

In some aspects, the present disclosure provides methods of promoting healthy neural development in a fetus, the methods comprising administering to a maternal subject gestating the fetus a composition comprising at least one metabolite selected from 3-indole sulfate, phenylacetylglycine, imidazole propionate, alpha-hydroxyisocaproate, 2-hydroxy-3-methylvalerate, alpha-hydroxyisovalerate, 1-methylhistamine, beta-hydroxisovalerate, 2R,3R-dihydroxybutyrate, and N-acetylleucine, or a combination thereof. In some aspects, the present disclosure provides methods of promoting healthy neural development in a fetus, the method comprising administering to a maternal subject gestating the fetus a plurality of catabolites of the branched chain amino acids (BCAAs), including alpha-hydroxyisocaproate, 2-hydroxy-3-methylvalerate, alpha-hydroxyisovalerate, beta-hydroxyisovalerate, and N-acetylleucine. In some aspects, the present disclosure provides methods of preventing impaired neural development in a fetus, the methods comprising administering to a maternal subject gestating the fetus a composition comprising at least one metabolite selected from 3-indole sulfate, phenylacetylglycine, imidazole propionate, alpha-hydroxyisocaproate, 2-hydroxy-3-methylvalerate, alpha-hydroxyisovalerate, 1-methylhistamine, beta-hydroxisovalerate, 2R,3R-dihydroxybutyrate, and N-acetylleucine, or a combination thereof. In some aspects, the present disclosure provides methods of preventing impaired neural development in a fetus, the methods comprising administering to a maternal subject gestating the fetus a plurality of catabolites of the branched chain amino acids (BCAAs), including alpha-hydroxyisocaproate, 2-hydroxy-3-methylvalerate, alpha-hydroxyisovalerate, beta-hydroxyisovalerate, and N-acetylleucine.

In some aspects, the present disclosure provides methods of treating impaired neural development in a fetus, the methods comprising administering to a maternal subject gestating the fetus a composition comprising at least one metabolite selected from 3-indole sulfate, phenylacetylglycine, imidazole propionate, alpha-hydroxyisocaproate, 2-hydroxy-3-methylvalerate, alpha-hydroxyisovalerate, 1-methylhistamine, beta-hydroxisovalerate, 2R,3R-dihydroxybutyrate, and N-acetylleucine, or a combination thereof. In some aspects, the present disclosure provides methods of treating impaired neural development in a fetus, the methods comprising administering to a maternal subject gestating the fetus a plurality of catabolites of the branched chain amino acids (BCAAs), including alpha-hydroxyisocaproate, 2-hydroxy-3-methylvalerate, alpha-hydroxyisovalerate, beta-hydroxyisovalerate, and N-acetylleucine.

In some embodiments, the composition comprises 3-indole sulfate. In some embodiments, the composition comprises phenylacetylglycine. In some embodiments, the composition comprises imidazole propionate. In some embodiments, the composition comprises alpha-hydroxyisocaproate. In some embodiments, the composition comprises 2-hydroxy-3-methylvalerate. In some embodiments, the composition comprises alpha-hydroxyisovalerate. In some embodiments, the composition comprises 1-methylhistamine. In some embodiments, the composition comprises beta-hydroxisovalerate. In some embodiments, the composition comprises 2R,3R-dihydroxybutyrate. In some embodiments, the composition comprises N-acetylleucine.

In some aspects, the present disclosure provides methods of promoting healthy neural development in a fetus, the methods comprising administering to a maternal subject gestating the fetus a composition comprising at least one short chain fatty acid selected from acetate, butyrate, and propionate, or a combination thereof.

In some aspects, the present disclosure provides methods of preventing impaired neural development in a fetus, the methods comprising administering to a maternal subject gestating the fetus a composition comprising at least one short chain fatty acid selected from acetate, butyrate, and propionate, or a combination thereof.

In some aspects, the present disclosure provides methods of treating impaired neural development in a fetus, the methods comprising administering to a maternal subject gestating the fetus a composition comprising at least one short chain fatty acid selected from acetate, butyrate, and propionate, or a combination thereof.

In some aspects, the present disclosure provides methods of promoting healthy neural development in a fetus, the methods comprising administering to a maternal subject gestating the fetus a plurality of catabolites of the branched chain amino acids (BCAAs).

In some aspects, the present disclosure provides methods of preventing impaired neural development in a fetus, the methods comprising administering to a maternal subject gestating the fetus a plurality of catabolites of the branched chain amino acids (BCAAs).

In some aspects, the present disclosure provides methods of treating impaired neural development in a fetus, the methods comprising administering to a maternal subject gestating the fetus a plurality of catabolites of the branched chain amino acids (BCAAs).

In some aspects, the present disclosure provides methods of promoting healthy neural development, the methods comprising administering to a maternal subject gestating the fetus a plurality of metabolites selected from Table 1. In some aspects, the present disclosure provides methods of preventing impaired neural development, the methods comprising administering to a maternal subject gestating the fetus a plurality of metabolites selected from Table 1. In some aspects, the present disclosure provides methods of treating impaired neural development, the methods comprising administering to a maternal subject gestating the fetus a plurality of metabolites selected from Table 1.

In some aspects, the present disclosure provides methods of promoting healthy neural development, the methods comprising modulating a plurality of metabolites selected from Table 1. In some aspects, the present disclosure provides methods of preventing impaired neural development, the methods comprising modulating a plurality of metabolites in the maternal subject or fetus selected from Table 1. In some aspects, the present disclosure provides methods of treating impaired neural development, the methods comprising modulating a plurality of metabolites in the maternal subject or fetus selected from Table 1.

TABLE 1
Differentially Regulated Metabolites in
PR Maternal Serum and PR Fetal Brain

| Downregulated Metabolites | Upregulated Metabolites |
|---|---|
| urea | 2-aminoheptanoate |
| beta- | S-methylcysteine sulfoxide |
| hydroxyisovaleroylcarnitine | |
| threonine | 1-methyl-5-imidazolelactate |
| 2R,3R-dihydroxybutyrate | dimethylglycine |
| N-acetylleucine | S-methylcysteine |
| argininate* | taurocyamine |
| imidazole propionate | 3-ureidoisobutyrate |
| 3-hydroxyisobutyrate | 2-aminooctanoate |
| beta-hydroxyisovalerate | glutarylcarnitine (C5-DC) |
| histamine | 2′-deoxyuridine |
| gamma-glutamylthreonine | carnitine |
| gamma-glutamylhistidine | sphingomyelin (d18:2/24:1, d18:1/24:2)* |
| N-acetylthreonine | myristoleoylcarnitine (C14:1)* |
| histidine | linoleoylcarnitine (C18:2)* |
| cystathionine | methionine sulfone |
| alpha-hydroxyisovalerate | 3-hydroxyhexanoylcarnitine (1) |
| isoleucine | 5-dodecenoylcarnitine (C12:1) |
| N-acetylisoleucine | laurylcarnitine (C12) |
| 1-methyl-4-imidazoleacetate | thymidine |
| 4-imidazoleacetate | 5,6-dihydrothymine |
| gamma-glutamylmethionine | tetradecadienoate (14:2)* |
| leucine | hexanoylglycine |
| isovalerylglycine | (S)-3-hydroxybutyrylcarnitine |
| N-acetylhistidine | N-acetylglycine |
| 2-aminoadipate | malonylcarnitine |
| cystine | 5,6-dihydrouracil |
| valine | 3-hydroxyoctanoylcarnitine (2) |
| alpha-hydroxyisocaproate | tauro-beta-muricholate |
| imidazole lactate | ophthalmate |
| 1-oleoyl-GPE (18:1) | N6,N6-dimethyllysine |
| methionine | palmitoyl sphingomyelin (d18:1/16:0) |
| cysteine | sphingomyelin (d18:2/16:0, d18:1/16:1)* |
| 1-methyl-4-imidazoleacetate | N,N,N-trimethyl-5-aminovalerate |
| 4-imidazoleacetate | 3-hydroxyoleoylcarnitine |
| gamma-glutamylmethionine | glycosyl-N-stearoyl-sphingosine |
| | (d18:1/18:0) |
| leucine | 5-methylthioribose** |
| isovalerylglycine | S-methylglutathione |
| N-acetylhistidine | cis-4-decenoylcarnitine (C10:1) |
| 2-aminoadipate | 3-hydroxyoctanoylcarnitine (1) |
| cystine | stearoyl sphingomyelin (d18:1/18:0) |
| valine | acetylcarnitine (C2) |
| alpha-hydroxyisocaproate | 5-aminovalerate |
| imidazole lactate | 2-aminoheptanoate |
| 1-oleoyl-GPE (18:1) | S-methylcysteine sulfoxide |
| methionine | 1-methyl-5-imidazoleacetate |
| docosatrienoate (22:3n6)* | 3-methylhistidine |
| 2-hydroxy-3-methylvalerate | 1-methyl-5-imidazolelactate |
| 2-palmitoylglycerol (16:0) | dimethylglycine |
| pyruvate | S-methylcysteine |
| 3-indoxyl sulfate | taurocyamine |
| 1-(1-enyl-palmitoyl)-GPE | 3-ureidoisobutyrate |
| (P-16:0)* | |
| phenylacetylglycine | 2-aminooctanoate |
| | glutarylcarnitine (C5-DC) |
| | 2′-deoxyuridine |
| | 3-ureidopropionate |
| | carnitine |
| | myristoylcarnitine (C14) |
| | sphingomyelin (d18:2/24:1, d18:1/24:2)* |
| | myristoleoylcarnitine (C14:1)* |
| | linoleoylcarnitine (C18:2)* |
| | methionine sulfone |
| | 3-hydroxyhexanoylcarnitine (1) |
| | 5-dodecenoylcarnitine (C12:1) |
| | laurylcarnitine (C12) |
| | thymidine |
| | stearoylcarnitine (C18) |
| | 5,6-dihydrothymine |
| | docosahexaenoylcarnitine (C22:6)* |
| | tetradecadienoate (14:2)* |
| | hexanoylglycine |
| | (S)-3-hydroxybutyrylcarnitine |
| | N-acetylglycine |
| | malonylcarnitine |
| | 5,6-dihydrouracil |
| | 3-hydroxyoctanoylcarnitine (2) |
| | tauro-beta-muricholate |
| | ophthalmate |
| | N6,N6-dimethyllysine |
| | palmitoyl sphingomyelin (d18:1/16:0) |
| | sphingomyelin (d18:2/16:0, d18:1/16:1)* |
| | N,N,N-trimethyl-5-aminovalerate |
| | 3-hydroxyoleoylcarnitine |
| | glycosyl-N-stearoyl-sphingosine |
| | (d18:1/18:0) |
| | 5-methylthioribose** |
| | S-methylglutathione |
| | cis-4-decenoylcarnitine (C10:1) |
| | 3-hydroxyoctanoylcarnitine (1) |
| | stearoyl sphingomyelin (d18:1/18:0) |
| | acetylcarnitine (C2) |
| | 1-oleoy1-2-linoleoyl-GPE (18:1/18:2)* |
| | 5-aminovalerate |
| | sphingomyelin (d18:1/24:1, d18:2/24:0)* |
| | (R)-3-hydroxybutyrylcarnitine |
| | sphingomyelin (d18:1/20:0, d16:1/22:0)* |
| | arabonate/xylonate |
| | 1-(1-enyl-palmitoyl)-2-palmitoyl-GPC (P- |
| | 16:0/16:0)* |
| | perfluorooctanesulfonate (PFOS) |
| | N,N-dimethyl-pro-pro |
| | cholesterol |

In some embodiments, the composition comprises acetate. In some embodiments, the composition comprises butyrate. In some embodiments, the composition comprises propionate.

In some embodiments, the maternal subject is undernourished. In some embodiments, the maternal subject is undernourished due to at least one of protein deficiency, poor weight gain, and fetal growth restriction.

In some embodiments, the subject is a mammal. In some embodiments, the subject is a human.

In some embodiments, the method comprises administering the composition at least once during the first trimester of the maternal subject's gestation period. In some embodiments, the method comprises administering the composition at least once during a period that runs from the start of the third week after conception to the end of the eighth week after conception. In some embodiments, the method comprises administering the composition at least once during a period that runs from the 17^{th} day post conception (dpc) to the 52^{nd} dpc. In some embodiments, the method comprises administering the composition at least once during the second trimester of the maternal subject's gestation period. In some embodiments, the method comprises administering the composition at least once during the third trimester of the maternal subject's gestation period.

In some embodiments, the fetus is an offspring of the maternal subject.

In some aspects, the present disclosure provides methods of conditioning a female subject for fostering healthy neural development in offspring, the methods comprising administering to the female subject a composition comprising at least one metabolite selected from 3-indole sulfate, phenylacetylglycine, imidazole propionate, alpha-hydroxyisocaproate, 2-hydroxy-3-methylvalerate, alpha-hydroxyisovalerate, 1-methylhistamine, beta-hydroxisovalerate, 2R,3R-dihydroxybutyrate, and N-acetylleucine, or a combination thereof; wherein the composition is administered at least once during a period that runs from the first day of an expected-but-missed menstruation to the end of gestation.

In some aspects, the present disclosure provides methods of conditioning a female subject for fostering healthy neural development in offspring, the methods comprising administering to the female subject a composition comprising at least one metabolite selected from short chain fatty acid selected from acetate, butyrate, and propionate, or a combination thereof; wherein the composition is administered at least once during a period that runs from the first day of an expected-but-missed menstruation to the end of gestation. In some embodiments, healthy neural development comprises healthy tactile sensory development. Healthy neural development can include healthy thalamocortical axon growth, healthy netrin-Gla+ thalamocortical axogenesis, healthy tactile sensory development, or a combination thereof. In some embodiments, healthy neural development comprises a reduction in anxiety-like behavioral deficits. In some embodiments, healthy neural development comprises prevention of learning and memory deficits.

The disclosed compositions can be administered at various times. For example, they can be administered at least once (e.g., once during the full period, twice during the full period, once a day) during a period that runs from the first day of an expected-but-missed menstruation to the end of gestation. Such timings can be useful to female subjects who prefer not to or cannot get tested for pregnancy though a professional facility. The administration time can also be at least once during a two-month period that ends with the day of an expected conception for the female subject. Such a timing can be useful for a subject who is planning pregnancy. The timing is, in some embodiments, at least once within the first trimester, second trimester, third trimester, or a combination thereof. More specific periods include the period that runs from the start of the third week after conception to the end of the eighth week after conception, and the period that runs from the 17^{th} dpc to the 52^{nd} dpc.

In various embodiments, offspring can include babies carried by a surrogate mother, in which the baby need not be the biological offspring of the gestating female.

In certain embodiments, the methods of the present disclosure are directed to promoting healthy, preventing impaired, or treating impaired neural development in a fetus, such as by administering to a maternal subject gestating the fetus (or to a female subject) a composition as described herein. Preferably, the method results in the fetus exhibiting a lesser degree of impaired neural development relative to a fetus gestated by similar a maternal subject (e.g., a maternal subject having a similar or identical maternal microbiome) not receiving the composition. Preferably, the method results in a prevention of anxiety-like behavioral deficits, anxiety, learning deficits, and/or memory deficits.

In additional embodiments of any of the aspects disclosed herein, the conjugate base forms or the conjugate acid forms of the disclosed compounds can be used, either instead of or together with their conjugate form. For example, in certain embodiments, imidazolepropionic acid can be used instead of or in addition to imidazole propionate.

In certain embodiments, the methods of the present disclosure are directed to inhibiting development of a disease or disorder in a fetus, e.g., by administering to a maternal subject gestating the fetus (or to a female subject) a composition as described herein. Preferably, the method results in the fetus exhibiting a lesser degree of development of the disease or disorder (e.g., a metabolic disorder, a cardiovascular disorder, a cerebrovascular disorder, stroke, Alzheimer's disease, schizophrenia, depression, or autism) during the fetal period and throughout the lifetime of the eventual child, adolescent, and adult, relative to a fetus gestated by a similar maternal subject (e.g., a maternal subject having a similar or identical maternal microbiome) not receiving the composition.

In certain embodiments, the methods further comprise administering the composition to the maternal subject or a female subject prior to gestation. In certain embodiments, the female subject is a fertile, ovulating female subject. In certain embodiments, the female subject is a female subject seeking to implant an embryo.

In certain embodiments, the composition comprises a compound selected from 3-indole sulfate, phenylacetylglycine, imidazole propionate, alpha-hydroxyisocaproate, 2-hydroxy-3-methylvalerate, alpha-hydroxyisovalerate, 1-methylhistamine, beta-hydroxisovalerate, 2R,3R-dihydroxybutyrate, N-acetylleucine, acetate, propionate, and butyrate, or a salt thereof, or a combination thereof.

Although maternal supplementation with 10 diet- and microbiome-dependent metabolites prevented impairments in particular anxiety-like and cognitive behavioral parameters in adult offspring, not all behavioral deficits induced by maternal protein restriction were prevented, suggesting that they are mediated by additional, as yet undefined, microbiome-dependent or -independent mechanisms. Results from cross-fostering experiments indicate that maternal protein restriction alters offspring behavior through mechanisms that require diet-induced programming of both fetal neurodevelopment and maternal physiology, as only offspring born to and fostered by dams on PR during gestation show behavioral deficits. One way in which maternal programming could translate is via maternal care behaviors, which are well-known to inform offspring neurodevelopment and behavior. Although significant differences in maternal retrieval behavior are not observed, which is altered in other gestational stress models, there may be changes in other domains of stress-sensitive maternal care that warrant further investigation, such as licking/grooming and nursing behaviors. Additionally, milk volume or nutrient content may be persistently altered by gestational PR, and may therefore create inconsistencies in postnatal nutrition between cross-fostering groups to inform long-term behavioral trajectories. Indeed, protein content of milk and size of offspring was reduced in dams fed a low protein diet during gestation and lactation, and sialylated milk oligosaccharides, found to be decreased in mothers of undernourished infants, were sufficient to promote growth in humanized mouse and pig models of early postnatal malnutrition. The impact of malnutrition and other peri-gestational stressors on both maternal and infant health underscores the importance of further research on women's health and investigation of lasting implications of the postpartum period.

Nutritional support, and even growth and gross physiological restoration, are often inadequate to prevent long-term microbial, neurological, and behavioral impairments caused by early malnutrition. Indeed, results from this study show consistent decoupling of early growth trajectories with later behavioral impairments. Restricting protein undernutrition to the pregnancy period in mice induces anxiety-like and impaired cognitive behavior in adult offspring that are fed standard nutritive diet since birth and exhibit typical growth trajectories by body weight. Depletion of the maternal microbiome exacerbates, whereas maternal supplementation with select microbes or microbially modulated metabolites prevents, select behavioral deficits in adult offspring without influencing pre- and postnatal growth. In contrast, maternal supplementation with short-chain fatty acids promotes feto-placental and offspring growth, but does not effectively prevent neurobehavioral deficits in adult offspring of dams that were protein restricted during pregnancy. Findings from this study reveal that the maternal microbiome modifies adverse neurobehavioral outcomes of gestational protein undernutrition, which are partially prevented by maternal supplementation with select microbial metabolites.

Applicant has shown herein that supplementing PR dams with ten diet- and microbiome-dependent metabolites during pregnancy decreases anxiety-like behavior and cognitive impairment in their adult offspring compared to vehicle-treated controls. Five of the ten metabolites (alpha-hydroxyisocaproate, 2-hydroxy-3-methylvalerate, alpha-hydroxyisovalerate, beta-hydroxyisovalerate, and N-acetylleucine) are dietary catabolites of the BCAAs leucine, isoleucine, and valine. The gut microbiota is capable of both producing and breaking down BCAAs, which are implicated in neural development and function following insult. Reduction of brain BCAAs by knocking out the transporter gene Slc7a5 was sufficient to induce severe neurological and behavioral impairments, and mutations in Slc7a5 have been tied to ASD and other neurological conditions in humans. Multiple impacted genes were identified in fetal brain related to BCAA degradation, which further supports their ability to interact directly with neurodevelopment to influence later behavior. In addition to these BCAA-related metabolites, 3-indoxyl sulfate, imidazole propionate, and phenylacetylglycine are well established microbial metabolites produced by sulfonation of bacterially-derived indole, direct bacterial synthesis from histidine, and bacterial conversion of phenylalanine, respectively, and have been implicated in neurodevelopment. The final 10M metabolites, 1-methylhistamine and 2R,3R-dihydroxybutyrate (4-deoxyerythronic acid), which are metabolites of histamine and L-threonine, respectively, have only correlational ties to the gut microbiome and brain.

### Sexual Dimorphism in Response to Protein Restriction

This study showed sexual dimorphism in behavioral responses to both PR alone, where female offspring showed a more severe anxiety-like phenotype, and to 10M intervention, where female offspring showed restoration of anxiety-like behavior, and male offspring showed restoration of memory deficits. These findings highlight the importance of examining both males and females and including sex as a biological variable. Sex biases have been well-documented in various neurodevelopmental conditions, including a male skew in autism spectrum disorder and attention deficit hyperactivity disorder and a female skew in anxiety and mood disorders. Furthermore, findings from this study support the ability of sex to influence susceptibility to, or presentation of, prenatal programming from environmental stressors or maternal microbial shifts. The biological basis for this interaction with sex is unclear, but theories have been raised to explain sexually dimorphic responses to prenatal perturbations, including slower maturation and increased intrauterine immunoreactive responses to male fetuses, differences in placental function by sex, and contributions of sex steroids and SRY programming. These different presentations of behavioral domain by sex also raise the question of different biological underpinnings of anxiety-like versus cognitive behaviors. Projections from the basolateral amygdala to the central amygdala and ventral hippocampus underlie anxiety-related behaviors, while spatial learning and memory rely on hippocampal CA3-medial prefrontal cortex circuits. Based on the sexually dimorphic and domain-specific behavioral responses of offspring treated with 10M during gestation, it may be the case that these interventions are acting in a sex- and circuit-specific manner to ameliorate select phenotypes of gestational protein restriction.

### Pharmaceutical Compositions

The compositions and methods of the present disclosure may be utilized to treat a subject in need thereof. In certain embodiments, the subject is a mammal such as a human, or a non-human mammal. When administered to subject, such as a human, the composition or the compound is preferably administered as a pharmaceutical composition comprising, for example, a compound of the disclosure and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the disclosure. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the disclosure. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of a subject without excessive toxicity, irritation, allergic response, or other problem or complication, commensurate with a reasonable benefit/risk ratio.

“Pharmaceutically acceptable salt” is used herein to refer to an acid addition salt or a basic addition salt which is suitable for or compatible with the treatment of patients.

The term “pharmaceutically acceptable acid addition salt” as used herein means any non-toxic organic or inorganic salt of the disclosed compounds. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, bitartaric, citric, ascorbic, maleic, benzoic, phenylacetic, cinnamic, salicylic, and sulfosalicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds disclosed herein are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds disclosed herein for laboratory use, or for subsequent conversion to a pharmaceutically acceptable acid addition salt.

The term “pharmaceutically acceptable basic addition salt” as used herein means any non-toxic organic or inorganic base addition salt of any acid compounds disclosed herein. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the subject being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the disclosure, with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Formulations of the disclosure suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present disclosure to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this disclosure. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, intraocular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions disclosed herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this disclosure, active compounds can be given per se or as a pharmaceutical composition containing, for example, about 0.1 to about 99.5% (more preferably, about 0.5 to about 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the subject being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the subject's condition, the disorder being treated, the stability of the compound, and, if desired, another type of therapeutic agent being administered with the compound of the disclosure. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods disclosed herein will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In some embodiments, the active compound may be administered two or three times daily. In some embodiments, the active compound will be administered once daily.

In some embodiments, compounds of the invention may be used alone or conjointly administered with another type of therapeutic agent.

In some embodiments, conjoint administration of compounds of the invention with one or more additional therapeutic agent(s) provides improved efficacy relative to each individual administration of the compound of the invention or the one or more additional therapeutic agent(s). In some such embodiments, the conjoint administration provides an additive effect, wherein an additive effect refers to the sum of each of the effects of individual administration of the compound of the disclosure and the one or more additional therapeutic agent(s).

This disclosure includes the use of pharmaceutically acceptable salts of compounds of the disclosure in the compositions and methods of the disclosure. In certain embodiments, contemplated salts include, but are not limited to, alkyl, dialkyl, trialkyl or tetra-alkyl ammonium salts. In certain embodiments, contemplated salts include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### EXAMPLES

### Example 1: Maternal Protein Restriction Disrupts Offspring Behaviour

Brain abnormalities, such as cerebral atrophy, ventricular dilation, and myelin-related deficits, are seen in protein malnourished infants as young as 3 months of age and through 36 months of age, highlighting an early critical period during which protein undernutrition impairs neurodevelopment. In animal models, restricting protein intake particularly during pregnancy yields persistent neurological and neurobehavioral impairments in the offspring, including abnormalities in neuronal proliferation and apoptosis, neocortical activity, hippocampal morphology, learning and memory, and anxiety-like behaviors. These results indicate that adverse neurological consequences of protein undernutrition can originate from gestational influences. In considering potential contributions of the microbiome, evidence from animal models indicate that alterations in the maternal microbiome contribute to adverse effects of immune activation, stress, and high-fat diet on neurological and behavioral deficits in the offspring, either by directing fetal neurodevelopment during pregnancy or by shaping early postnatal neural development via vertical transmission at birth and postpartum. These findings align with human studies reporting that alterations in the maternal microbiome during pregnancy are associated with abnormalities in offspring behavior and that postpartum nursing supports cognitive, language, and microbiome development in the first years of life. However, mechanisms by which maternal protein undernutrition leads to lasting neurobehavioral deficits in the offspring, and how these processes may be modified by the microbiome, are unknown.

Herein, the effects of maternal protein undernutrition during pregnancy on maternal-fetal health and offspring behavior in mice are examined. A cross-fostering paradigm is used to evaluate the ability of maternal protein restriction to directly alter fetal neurodevelopment and indirectly condition maternal physiology to engender lasting cognitive and anxiety-related behaviors in adult offspring. Effects of maternal protein restriction on the maternal microbiome are profiled, and the impact of maternal microbiome depletion and microbial metabolite supplementation on maternal-fetal and offspring behavioral responses to maternal protein restriction is assessed. Results from this study highlight the importance of the gestational period in protein undernutrition altering both maternal health and fetal developmental trajectories. Furthermore, results reveal a role for the maternal microbiome in modulating the severity of fetal developmental and adult neurobehavioral impairments induced by maternal protein undernutrition. These advances in illuminating molecular underpinnings of adverse behavioral outcomes of protein undernutrition could potentially lead to new approaches to ameliorate neurological disorders that co-occur with impaired growth in malnourished children.

### Methods

### Mice

8-week old male and female C57Bl/6J mice were purchased from Jackson Laboratories and maintained on 12-h light-dark cycle in temperature- and humidity-controlled environment. All mice were kept under sterile conditions (autoclaved cages, bedding, water bottles, and water). All experiments were performed in accordance with the NIH Guide for the Care and Use of Laboratory Animals using protocols approved by the Institutional Animal Care and Use Committee at UCLA.

### Protein Restriction

Mice were first subjected to a microbiota normalization, where two scoops of bedding were removed from each cage, mixed together, and deposited back in each cage to normalize microbiota between all mice in each testing cohort. At least 5 days after normalization, male and female mice were given either 6% protein diet (Teklad Envigo; TD.90016) or 20% control diet (Teklad Envigo; TD.91352) ad libitum for at least 2 weeks. Following this habituation period, mice were paired and time-mated. At E0.5, determined by observation of copulation plug, pregnant dams were individually housed and continued to be fed the same diet through gestation. Weights and fecal samples were collected from each dam at E0.5 and 18.5, with a subset collected at E0.5, 7.5, 10.5, 14.5, and 18.5.

### Antibiotic Treatment

For “ABX” treatment groups, SPF male and female mice were gavaged twice daily for one week with neomycin (100 mg/kg), metronidazole (100 mg/kg) and vancomycin (50 mg/kg), as previously described. During this time, ampicillin (1 mg/mL) was provided ad libitum in drinking water. Mice were then time-mated as described above. Following gavage, mice were maintained on ampicillin (1 mg/mL), neomycin (1 mg/mL) and vancomycin (0.5 mg/mL) ad libitum in drinking water for the duration of the experiment.

### Fetal Tissue Collections

At E18.5, dams were sacrificed by cervical dislocation. Fecal samples were extracted from the colon. Blood was collected by cardiac puncture into vacutainer SST tubes (Becton Dickinson) and allowed to clot for 1 hour at room temperature, before centrifuging at 1500×g, 4° C. for 10 minutes. Serum supernatant was collected and stored at −20° C. The entire uterine horn, including all conceptuses, was removed and placed in ice cold 1×PBS. Dams were weighed once again to record post-transection weight. Each fetus was dissected from the amniotic sac and weighed. Brains were microdissected and either placed into RNAlater (Invitrogen) for subsequent RNA sequencing, or snap-frozen in liquid nitrogen for subsequent metabolomics analysis. All samples were stored at −80° C.

### Behavioral Testing

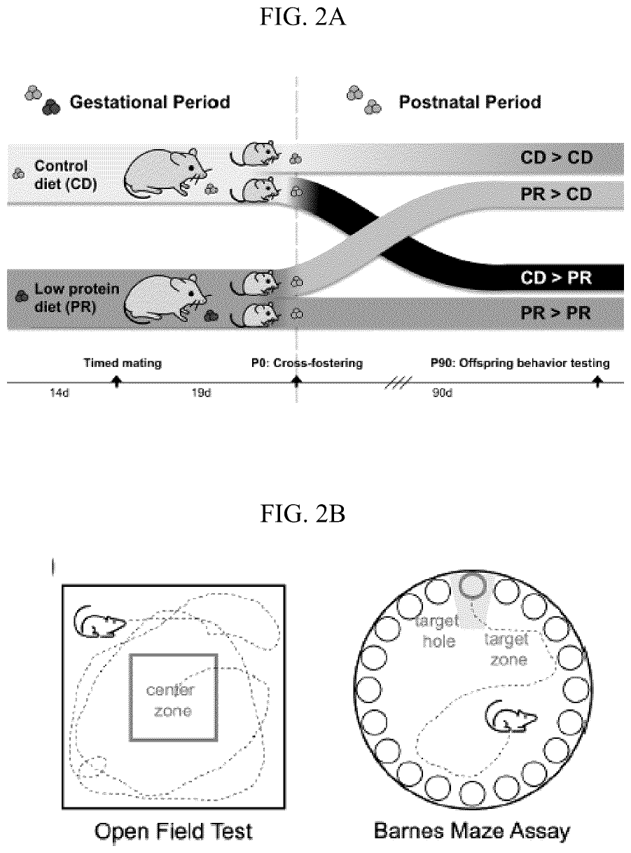

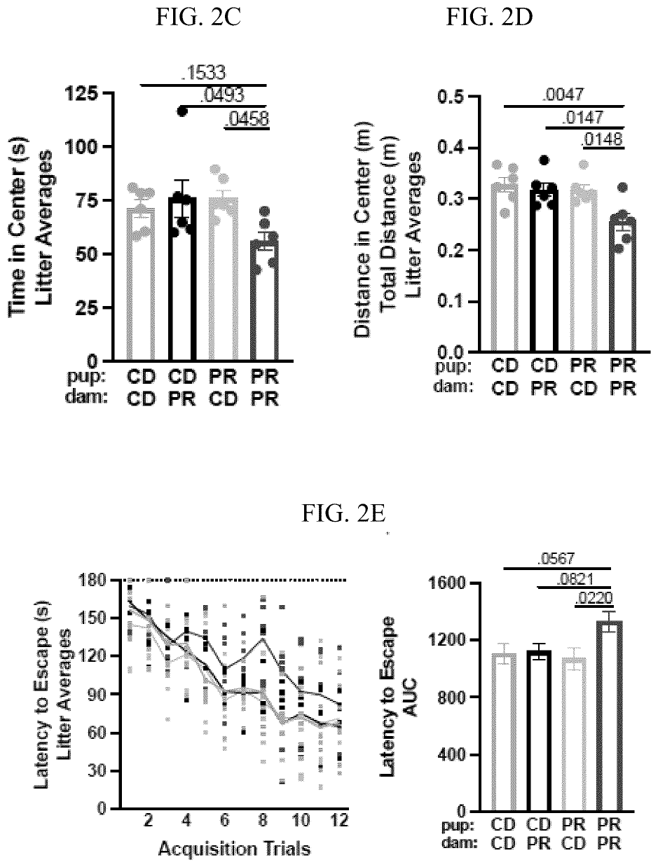

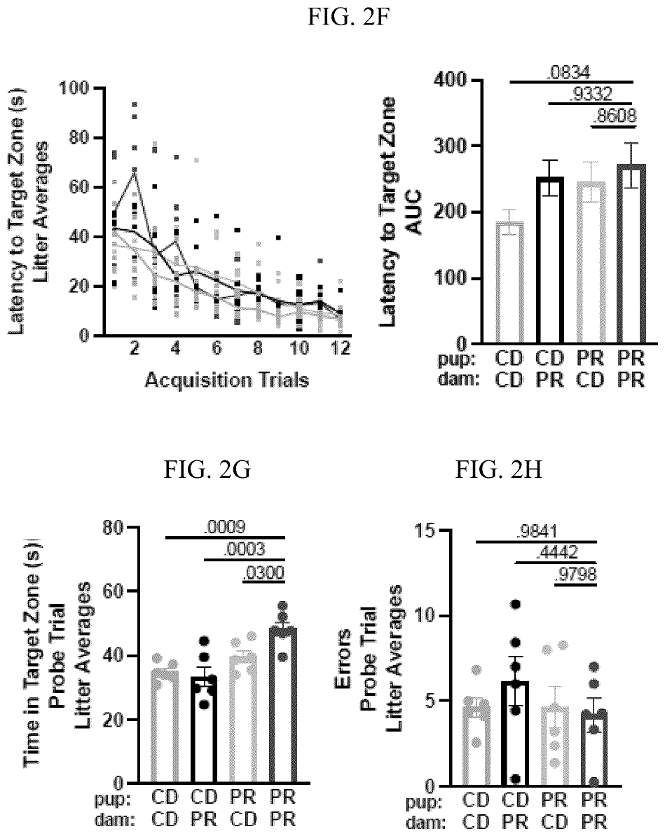

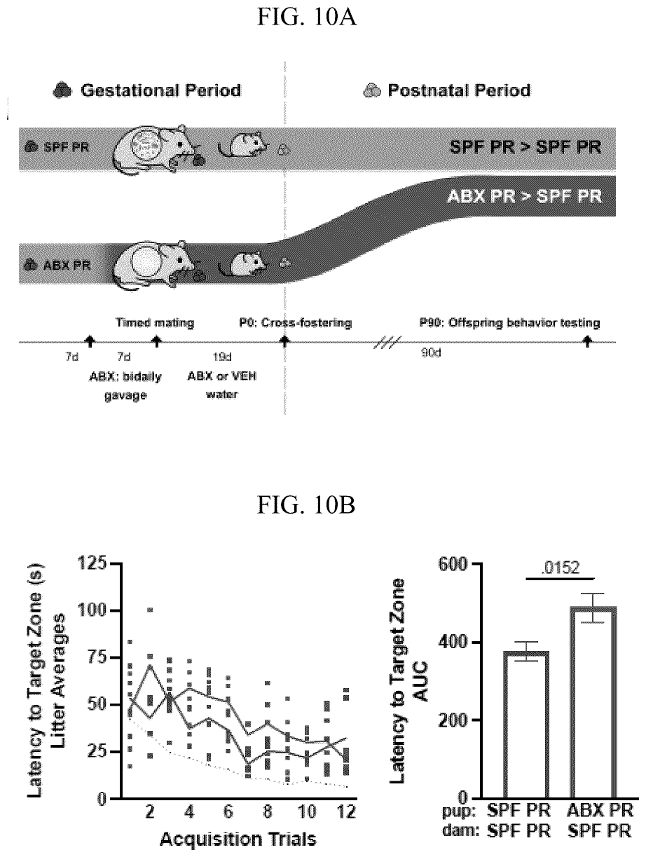

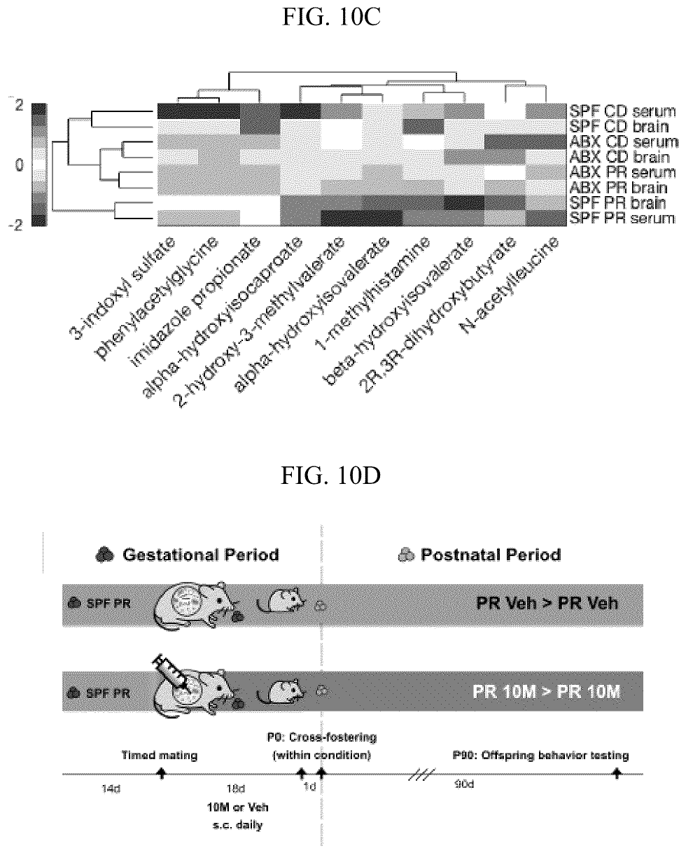

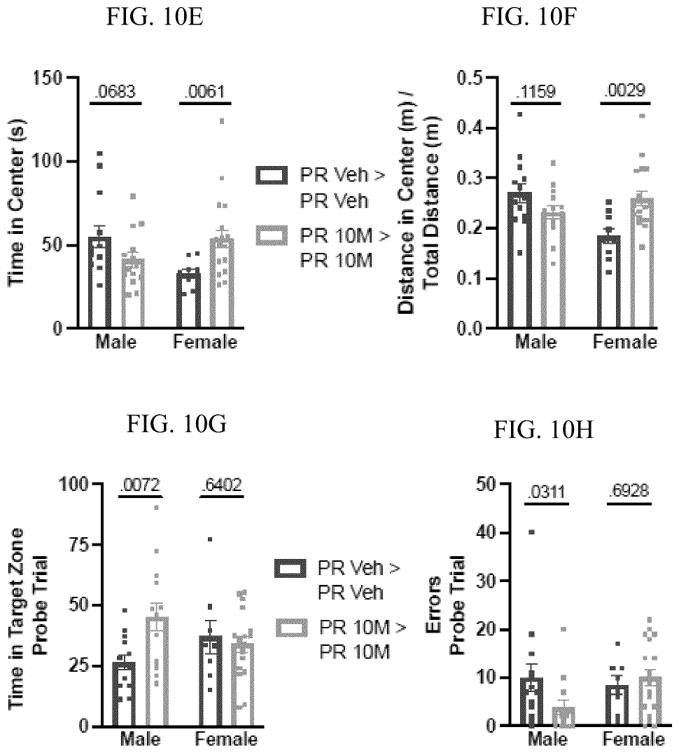

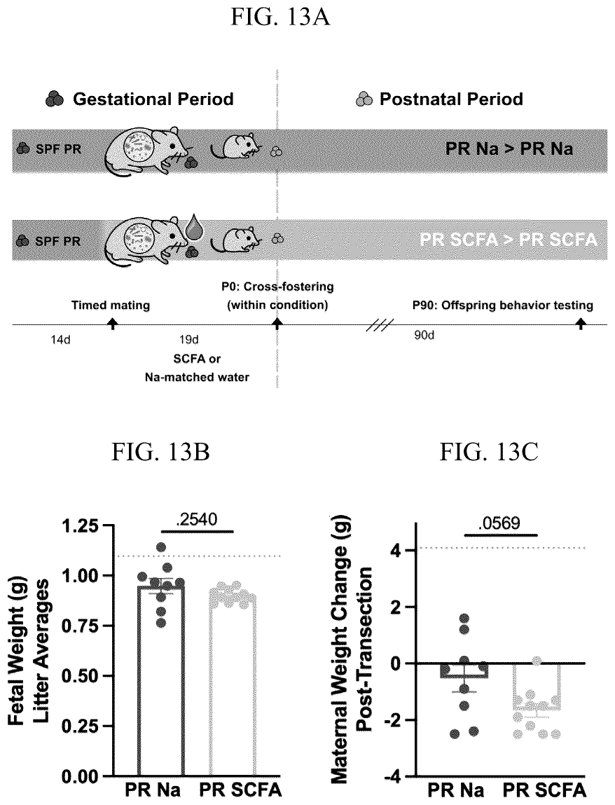

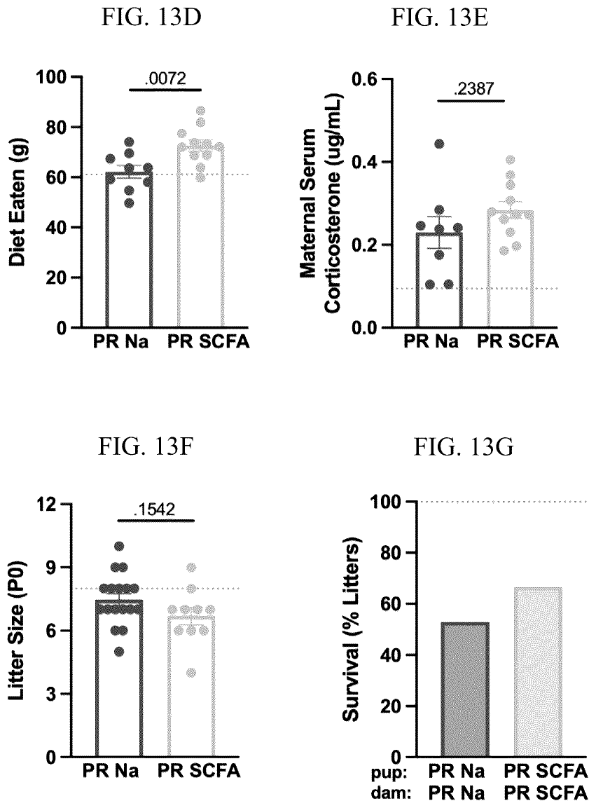

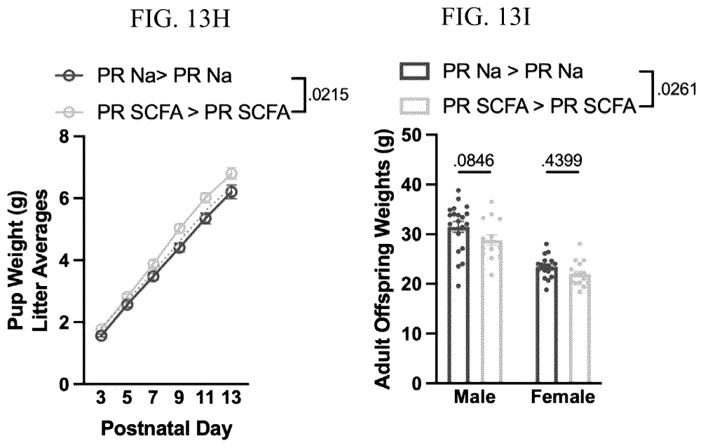

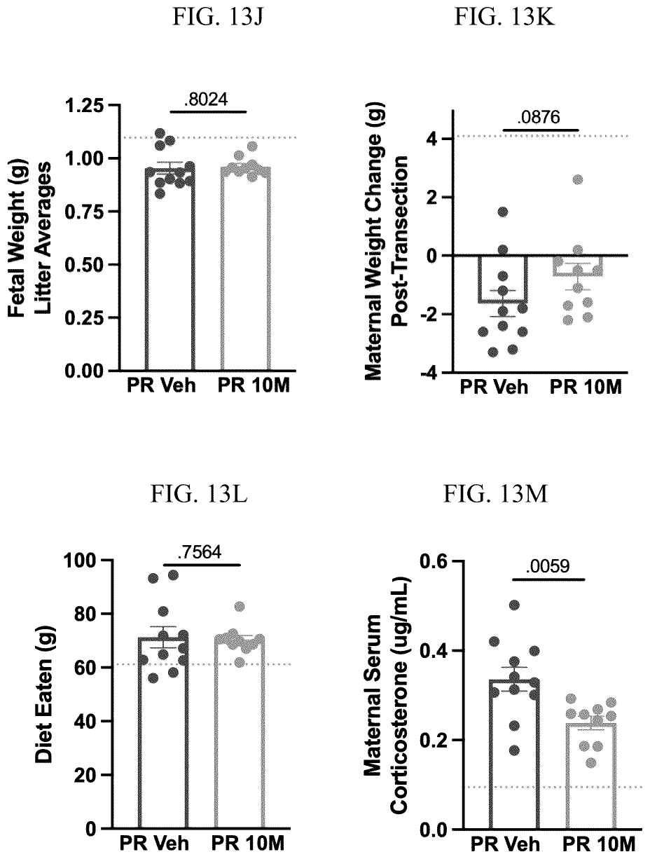

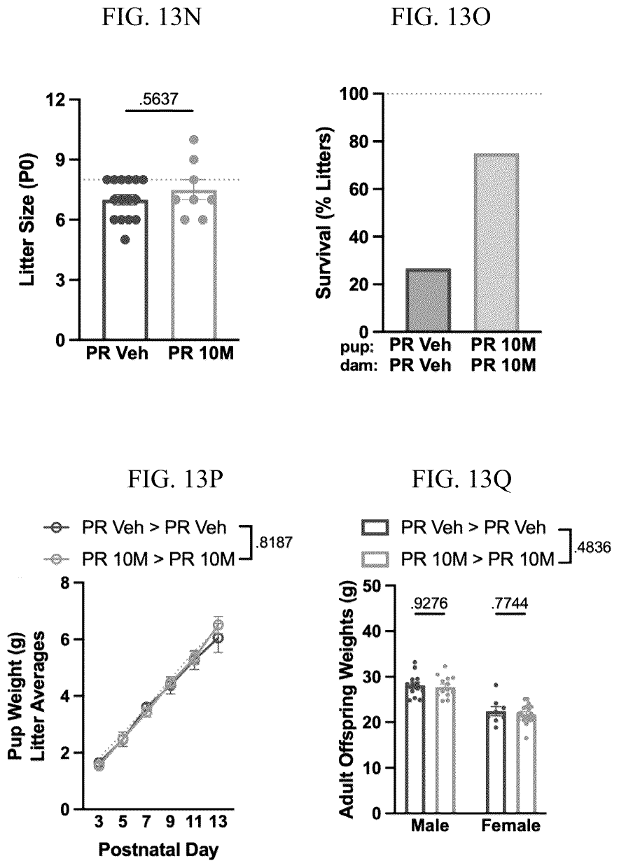

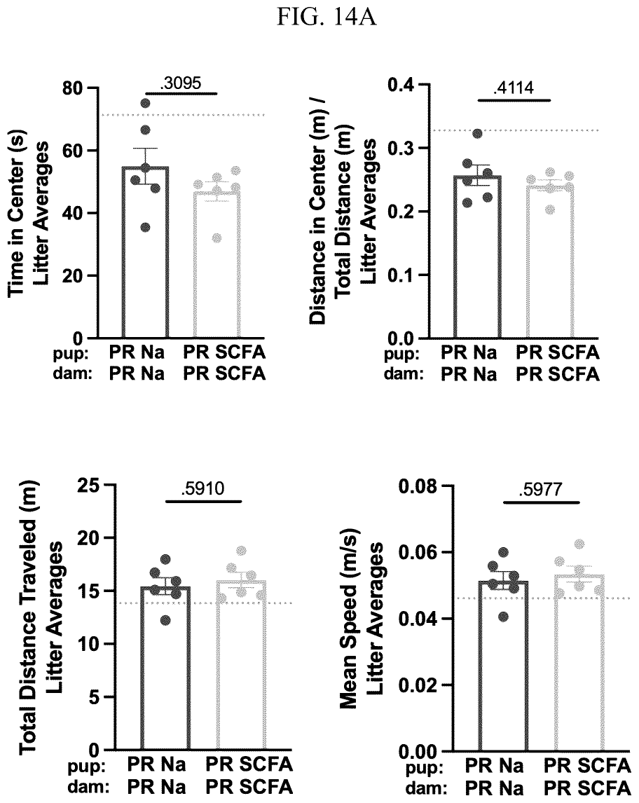

For behavioral cohorts, dams were given shepherd shacks on E17.5 to reduce stress ahead of birth. At P0, dams and diet were weighed, pups were counted, and all diet groups were switched to CD. Complete litters were cross-fostered, as denoted in figures (FIG. 2A, FIG. 10A,D, FIG. 13A). Subsequently, litters were checked daily for pup survival. Litters were denoted as not survived if <4 pups remained at weaning (as previously done). Pups were weighed on P3, 5, 7, 9, 11, and 13, and toe-tattooed P4-P6, for identification purposes. Pups were weaned at P24-P26, and separated into cages with same-sex littermates. Adult behaviors were run beginning when mice were −90 days old. All mice were habituated in the testing room 30 minutes prior to test start. Tests were run in order of least to most stressful, in the order described below, with at least 3 days separating each test. All equipment was cleaned with 70% ethanol and allowed to dry in between trials.

### Righting Reflex & Maternal Retrieval—SPF Cohorts Only

The righting reflex and maternal retrieval tests were utilized to examine early neurological development and maternal care-giving and responsiveness, respectively. Pups were subjected to righting reflex and maternal retrieval tests on P3, 5, 9, and 11. All pups from each litter were placed one-by-one on their backs and the time it took for them to right themselves (with all four paws on the ground) was recorded, capped at 30 seconds. Pups were then placed back into their home cage all at once, in the opposite corner of the nest. The latency to first pup retrieval and full litter retrieval by the dam was measured, capped at 5 minutes.

### Ultrasonic Vocalizations—SPF Cohorts Only

Ultrasonic vocalizations in pups were analyzed as a response to separation from the litter. Pups were tested for USVs at P7 and P13. Four pups were randomly chosen (when possible, 2 males and 2 females) for USV testing, and the same pups were used at both timepoints. Pups were habituated in the recording chambers for 5 minutes, and then recorded for 5 minutes. Number and duration of calls, in addition to inter-call latency, were analyzed. Recording and analysis were performed using SASLab Pro (Avisoft Bioacoustics).

### Open Field Test—All Cohorts

Open field test was employed to assess locomotion and thigmotaxis as a proxy for anxiety-like behavior. Mice were placed into open field arenas (27.5 cm²) for 10 minutes under bright light. Videos were recorded using an overhead camera, and the first 5 minutes of each test were analyzed using AnyMaze 7.1 for time spent in center zone, distance traveled in center zone/total distance traveled, total distance traveled, and mean speed.

### Barnes Maze—All Cohorts

Barnes maze was employed to assess spatial learning and memory. The Barnes maze apparatus has an elevated round surface (90 cm diameter) with 20 holes evenly spaced around the perimeter. One hole was denoted as the “escape” hole, and an escape box was attached below the hole. The position of the escape hole was kept consistent within mice, but counterbalanced between mice. Spatial cues (black and white geometric shapes printed on 9×11 in paper) were placed around the maze, and kept consistent throughout training and testing. On day 1 of the testing protocol, each mouse was allowed to freely explore the Barnes maze apparatus for 5 minutes with no white noise under mild light, with no escape box present. Immediately following, each mouse was placed under a wire cup in the center of the apparatus, with white noise and bright light for 1 minute. Finally, each mouse was gently guided to the escape hole and into the escape box, and white noise and bright light were removed, for 1 minute. On days 2-5, each mouse was subject to three training trials, with an inter-trial latency of 20-45 minutes. The training trials consisted of 15 s habituation, where mice were placed under a wire cup in the center of the apparatus with bright light and white noise. The cup was then lifted, and mice were given 180 s to escape into the escape hole, thereby ending the test. If the mouse did not escape by 180 s, they were gently guided into the escape hole.

At the end of each trial, mice were left for 1 minute in the escape hole, with no white noise. On day 6, a probe test was run to assess long-term memory. Each mouse was subject to one probe trial, consisting of a 15 s habituation, and then a 180 s test without the escape box. Videos were recorded using an overhead camera and analyzed in AnyMaze 7.1. For the training trials, latency to find the escape hole and latency to enter the escape box were analyzed. For the probe trial, time spent in the escape hole vicinity, and errors, defined as non-escape hole vicinities a mouse entered before entering the escape hole vicinity, were analyzed.

### Human Infants

This prospective cohort study was conducted at the University of California Los Angeles (UCLA) neonatal intensive care units (UCLA Ronald Reagan Hospital/UCLA Mattel Children's Hospital, Los Angeles, CA and UCLA Santa Monica Hospital, Santa Monica, CA). The UCLA Institutional Review Board granted approval for this study (IRB #15 00718). Verbal informed consent was obtained from parents/legal guardians. Inclusion criteria included infants with a birth weight<2 kilograms (kg), <14 days of age, who were predicted to require parenteral nutrition for >2 weeks, and follow-up at the UCLA High Risk Infant Clinic (HRIF). The exclusion criteria included infants who were unlikely to survive and infants with congenital anomalies and congenital infections. Infants missing prenatal medical records with which to diagnose FGR were also excluded.

Demographic and clinical data was collected from the electronic medical chart. FGR was determined as previously described by the obstetric team who documented fetal growth deceleration on repeated prenatal ultrasounds. Full feeds were defined as 100 mL/kg/d of enteral nutrition or ad libitum feeding, whichever occurred first. Early onset sepsis was defined as a positive blood culture before 72 hours of age and antibiotics for >5 days. Late onset sepsis was defined as a positive blood culture after 72 hours of age and antibiotics for >5 days. Necrotizing enterocolitis was defined by Bell's stage II or III. Neurodevelopment was assessed using composite cognitive, language, and motor scores from the Bayley Scales of Infant Development (BSID) III, which was conducted by doctorally trained clinicians who work in the HRIF. These clinicians have established inter-rater reliability for the BSIS-III examination, which is monitored for recertification research projects and clinical assessments.

16S rRNA Gene Sequencing

Fecal samples were collected from CD and PR dams throughout gestation at the following timepoints: E0.5, E7.5, E10.5, E14.5, E18.5 and stored at −80° C. until processing. The same dams were collected from at each timepoint. Bacterial genomic DNA was isolated according to manufacturer instructions using the DNeasy PowerSoil Kit (Qiagen). Additionally, fecal samples were collected from preterm infants with and without FGR during their hospital stay. Specimens were collected shortly after study enrollment, then weekly while on parenteral nutrition, and for four weeks after parenteral nutrition was discontinued. Samples were collected from infant diapers using sterile collection kits and stored at −80° C. until processing. Because of intestinal dysmotility, preterm infants have delayed passage of meconium and do not stool frequently in the first couple weeks of life, resulting in missing samples disproportionately at early timepoints. Approximately 50 mg per sample was aliquoted in liquid nitrogen, bead-beat in buffer RLT using Lysing Matrix E tubes (MP Biomedicals), and extracted using AllPrep DNA/RNA/Protein Mini kit (Qiagen), as previously described.

For both mouse and human samples, 16S rRNA gene sequencing was performed as previously described. Briefly, sequencing libraries were generated according to methods adapted from Caporaso et al. 2011, amplifying the V4 regions of the 16S rRNA gene by PCR. The final PCR product was purified using the Qiaquick PCR purification kit (Qiagen). 250 ng of purified, PCR product from each individually barcoded sample were pooled and sequenced by Laragen, Inc. using the Illumina MiSeq platform and 2×250 bp reagent kit for paired-end sequencing. All analyses were performed using QIIME2 2023.7, including DADA2 for quality control, taxonomy assignment, alpha-rarefaction and beta-diversity analyses. 29,152 reads were analyzed per sample. Operational taxonomic units (OTUs) were assigned based on 99% sequence similarity compared to the SILVA 138 database. Beta-diversity principal coordinates analysis plots were generated with QIIME2 View, and other bacterial metrics, including correlation analyses with clinical metadata were performed and plotted in Prism 9.0 (Graphpad).

### Corticosterone ELISAs

Total corticosterone levels were determined by a corticosterone ELISA assay (Enzo Life Sciences). The assay was run per manufacturer's instructions, following the small volume protocol. All experimental samples were run in triplicate; blanks and controls were run in duplicate. Mean optical density was read on a Synergy H1 plate reader (Agilent BioTek), and corticosterone concentrations were determined in ug/mL based on a standard curve.

### Fetal Brain RNA Sequencing

Whole fetal brains were randomly chosen from each litter, dissected, and immediately placed into 400 uL RNAlater (ThermoFisher Scientific) and stored at −80° C. RNA was extracted and cDNA synthesized using the PureLink RNA Mini Kit (Invitrogen) and qScript cDNA synthesis kit (Quantabio), respectively. Equal amounts of RNA were pooled from 2 brains per litter, resulting in 1522 ng RNA per sample, except for one which had 508 ng. An RNA integrity number (RIN) of at least 8.7 was confirmed for each sample using the 4200 Tapestation system (Agilent). RNA was prepared as previously described using the TruSeq RNA Library Prep kit and Lexogen QuantSeq 3′ forward-sequencing was performed using the Illumina HiSeq 4000 platform by the UCLA Neuroscience Genomics Core. Sequences were quality filtered, trimmed, and mapped using FastQC v0.11.5, bbduk v35.92, and RSeQC v2.6.4. Reads were aligned to UCSC Genome Browser assembly ID: mm10 using STAR v2.5.2a, indexed using samtools v1.3, and aligned using HTSeq-count v0.6.0. Differential expression analysis was conducted using DESeq2 v1.24.041. Heatmaps were generated using the pheatmap v1.0.12 package for R. DAVID 2021 was used to conduct GO term enrichment analysis of differentially expressed genes with non-adjusted p-value<0.05.

### Metabolomics

Whole fetal brains were randomly chosen from each litter, dissected, and immediately snap-frozen in liquid nitrogen and stored at −80° C. 1.5-2 brains were pooled for subsequent analysis. Untargeted metabolomics was run on fetal brain and maternal serum by Metabolon Inc. as previously described. Briefly, samples were prepared using the automated MicroLab STAR system (Hamilton Company) and analyzed on GC/MS, LC/MS and LC/MS/MS platforms. Organic aqueous solvents were used to perform serial extractions for protein fractions, concentrated using a TurboVap system (Zymark) and vacuum dried. For LC/MS and LC-MS/MS, samples were reconstituted in acidic or basic LC-compatible solvents containing >11 injection 25 standards and run on a Waters ACQUITY UPLC and Thermo-Finnigan LTQ mass spectrometer, with a linear ion-trap frontend and a Fourier transform ion cyclotron resonance mass spectrometer back-end. For GC/MS, samples were derivatized under dried nitrogen using bistrimethyl-silyl-trifluoroacetamide and analyzed on a Thermo-Finnigan Trace DSQ fast-scanning single-quadrupole mass spectrometer using electron impact ionization. Chemical entities were identified 30 by comparison to metabolomic library entries of purified standards. Following log transformation and imputation with minimum observed values for each compound, data were analyzed using two-way ANOVA to test for group effects. P- and q-values were calculated based on two-way ANOVA contrasts. Principal components analysis was used to visualize variance distribution with the ggplot2 R package. The Metaboanalyst 5.0 platform's metabolite set enrichment analysis (MSEA) was performed on whole fetal brain and maternal serum metabolites that were statistically significantly altered (non-adjusted p-value<0.05) between SPF PR and SPF CD treatment groups and ABX PR and SPF PR treatment groups. Metabolites sets were analyzed for metabolite pathway enrichment using the Small Molecule Pathway Database (SMPDB).

### In Vivo Short-Chain Fatty Acid Supplementation

All mice were habituated on PR diet, and bred as described above. Once copulation plugs were observed, SCFA were supplemented ad libitum in drinking water from E0.5-E18.5 (prenatal cohort) or E19.5 (behavior cohort), as previously described. SCFA water cocktail contained 25 mM sodium propionate, 40 mM sodium butyrate, and 67.5 mM sodium acetate. SCFA water was changed at least every 4 days. Sodium-matched controls were given water supplemented with 7.745 g/L sodium chloride. All supplemented water was sterile-filtered before administration. SCFA and Na-matched litters were then used for E18.5 tissue collections, or for postnatal cross-fostering and behavioral testing, as described above.

### In Vivo Metabolite Supplementation

### Metabolite Selection for In Vivo Supplementation

Metabolites were first filtered for significant differences between SPF CD and SPF PR. Those that were decreased in SPF PR compared to SPF CD were further checked for microbiome modulation, as denoted by significant change in any direction in ABX PR relative to SPF PR, or in ABX CD relative to SPF CD. This set of filters was applied to metabolomic results from both fetal brain and maternal serum. Metabolites that met these criteria from both tissue compartments were used for supplementation.

### Metabolite Supplementation

All mice were habituated on PR diet, and bred as described above. Once copulation plugs were observed, sterile filtered metabolite mixture (10M) was injected subcutaneously once per day from E0.5-E17.5. Control dams were injected with vehicle. Physiological concentrations were determined as previously described based on literature, estimated blood volume, and on the relative differences in metabolite fold change between CD and PR dams (Tables 3-4). The 10M injections contained 0.76 ug imidazole propionate, 7.3 ug alpha-hydroxyisocaproate, 7.24 ug alpha-hydroxyisovalerate, 0.0094 uL beta-hydroxyisovalerate, 11.68 ug N-acetylleucine, 2.16 ug 2-hydroxy-3-methylvalerate, 6.3 ug phenylacetylglycine, 29.16 ug 3-indoxyl sulfate, 0.04 ug 1-methylhistamine, and 6.66 ug 2R,3R-dihydroxybutyrate in 200 uL 1× sterile PBS. Vehicle injections contained 8.66 ug potassium chloride, 0.012 nL hydrochloric acid, and 1.7 ug sodium hydroxide in 200 uL 1× sterile PBS. 10M and vehicle litters were then used for E18.5 tissue collections, or for postnatal cross-fostering and behavioral testing, as described above.

### Statistical Methods and Sample Sizes

Statistical analyses were performed using Prism 9.0 (Graphpad). Assays with two groups were assessed for normality and subsequently analyzed by either an unpaired two-tailed t-test with Welch's correction, or by a Mann-Whitney test. Tests on three or more groups were assessed by a one-way ANOVA, and when there were two factors, with a two-way ANOVA. Data over time were assessed with a repeated measures ANOVA or mixed measures analysis. Tukey or Sidak post-hoc tests were used, based on Prism recommendations. All prenatal weight measures and postnatal behavioral measures were run using litter as a biological replicate, with all fetuses or offspring averaged within each litter. For prenatal weights and maternal metrics, at least 9 litters per group were analyzed. For behavioral experiments, 4-7 litters per group were tested. To assess sex differences in the behavioral results, and in adult weights, individual male and female offspring were treated as biological replicates. For metabolomics, 1.5-2 fetal brains were randomly chosen and pooled per litter, from 6 litters per group. For RNAseq, 2 fetal brains were randomly chosen and pooled per litter, from 5 litters per group. Whole litters were excluded from all analyses if they had fewer than 4 fetuses or pups. For prenatal measures, no viable fetuses within any litters were excluded from reported analyses. For behavioral measures, individual offspring were occasionally excluded from all tests due to health reasons (i.e. developing malocclusion) or from a specific test or trial due to a test-related event (i.e. escaping the testing chamber). These occasions were rare, and not associated with a particular group or condition. All data are plotted as mean+/−SEM. Significant differences are denoted as follows: *p<0.05, **p<0.01, ***p<0.001, ****p<0.0001, n.s. not significant.

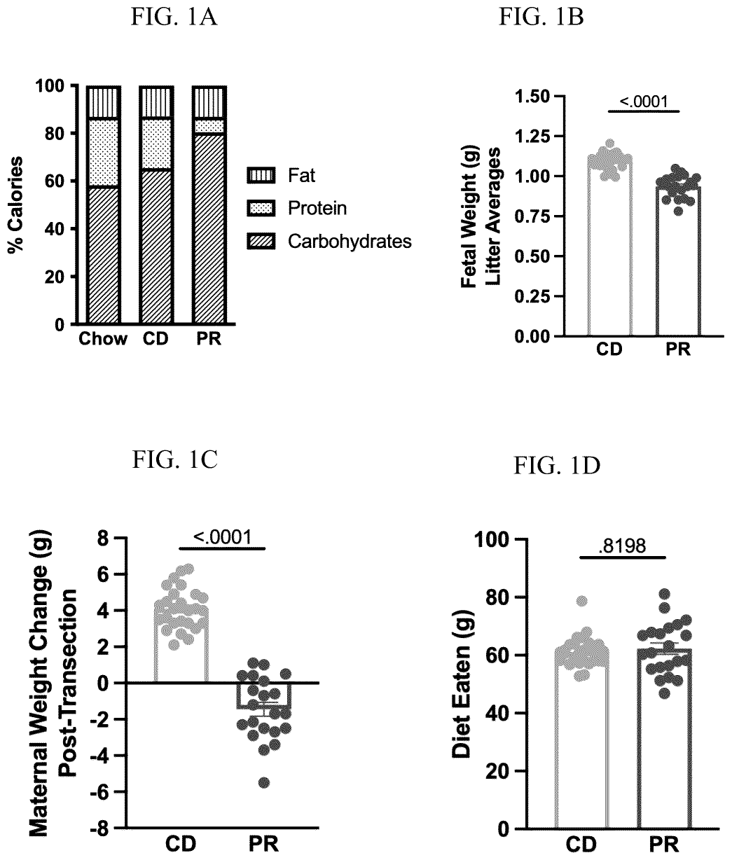

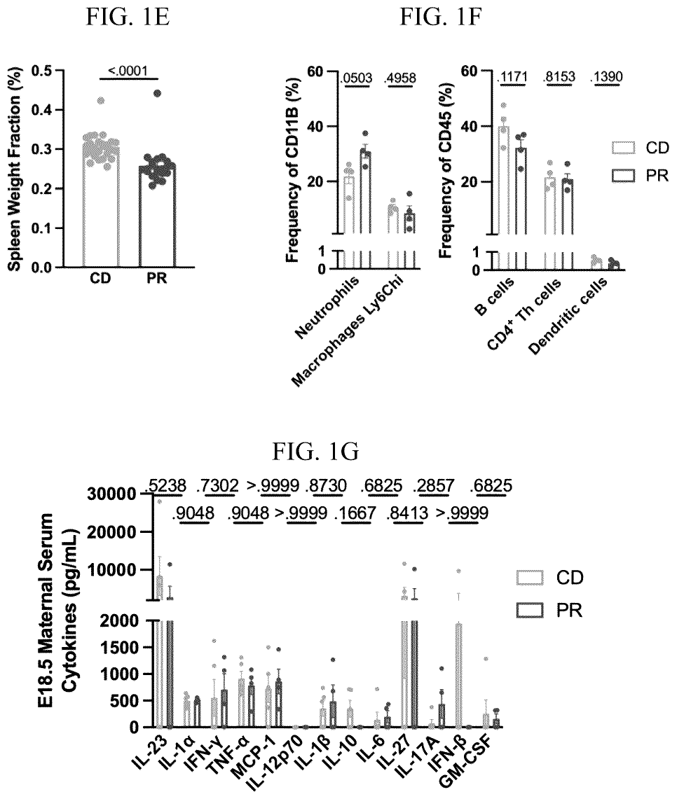

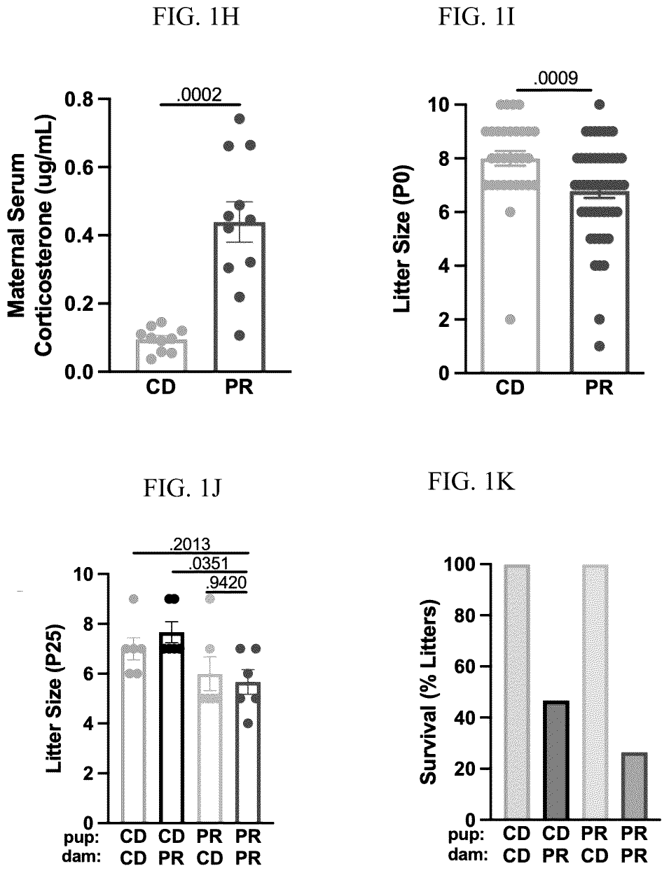

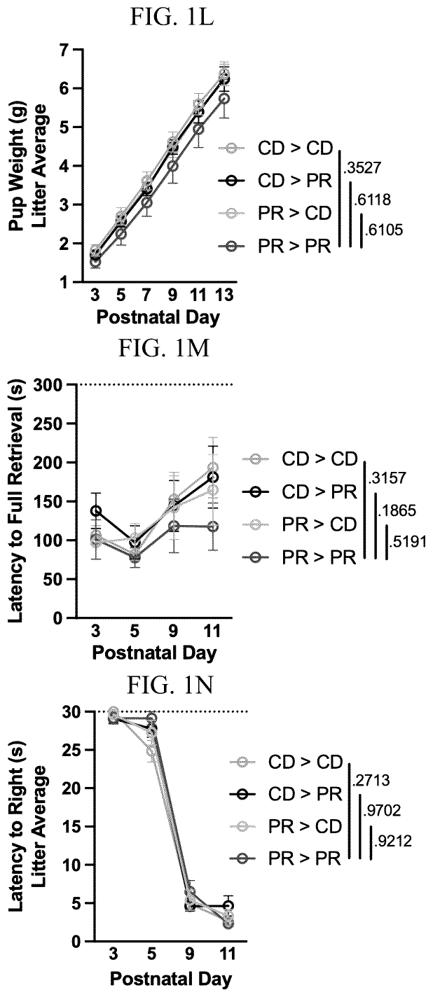

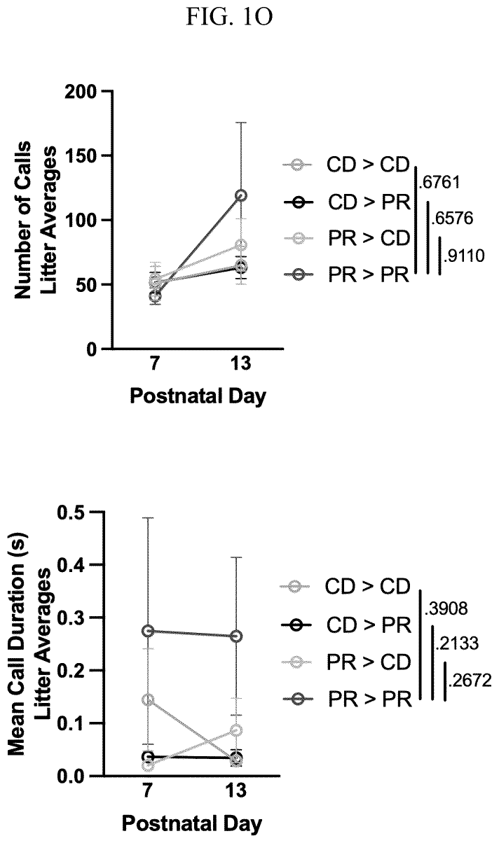

Protein undernutrition is associated with both childhood stunting and neurological dysfunction, but the etiopathogenesis of lasting neurobehavioral deficits remains poorly understood. To explore this relationship, effects of maternal protein restriction particularly during pregnancy on behavior in adult offspring were evaluated. To model maternal protein undernutrition, male and female C57Bl/6J mice were fed a 6% protein diet (protein restriction, PR) or 20% protein control diet (CD) for 2 weeks prior to timed-mating and throughout gestation (FIG. 2A). PR and CD formulations were isocaloric, where the 14% protein content lacking in PR was replaced by carbohydrates, mainly sucrose and cellulose (FIG. 1A, Table 2). Consistent with existing literature using this paradigm as a model of fetal growth restriction (FGR), maternal consumption of a PR diet prior to and throughout pregnancy resulted in reduced fetal weight at late gestation (embryonic day (E) 18.5) and maternal net weight loss with no difference in diet consumption, in addition to elevated maternal serum corticosterone and reduced litter size at birth (FIG. 1B-F). PR elevated maternal serum corticosterone and reduced litter size at birth (FIG. 1H-I). PR dams displayed significantly smaller spleens (FIG. 1E) and greater frequency of splenic neutrophils (FIG. 1F), but no difference in serum cytokines (FIG. 1G). These findings suggest a state of maternal physiological stress and immune dysregulation.

TABLE 2
Nutritional information for control and low protein diets

| | 20% Protein | | 6% Protein | |
|---|---|---|---|---|
| | Diet (CD) | | Diet (PR) | |
| Nutrient | g/Kg | | g/Kg | |
| Casein | 230 | | 69 | |
| DL-Methionine | 3 | | 0.9 | |
| Sucrose | 431.7 | | 571.8 | |
| Corn Starch | 200 | | 200 | |
| Corn Oil | 52.3 | | 53.9 | |
| Cellulose | 37.86 | | 57.82 | |
| Vitamin Mix, Teklad (40060) | 10 | | 10 | |
| Ethoxyquin, antioxidant | 0.01 | | 0.01 | |
| Mineral Mix, Ca—P Deficient | 13.37 | | 13.37 | |
| (79055) | | | | |
| Calcium Phosphate, dibasic | 16.66 | | 21.6 | |
| Calcium Carbonate | 5.1 | | 1.6 | |
| | % by | % kcal | % by | % kcal |
| | weight | from | weight | from |
| Protein | 20.3 | 21.6 | 6.1 | 6.5 |
| Carbohydrate | 61.6 | 65.4 | 75.6 | 80.4 |
| Fat | 5.5 | 13 | 5.5 | 13.1 |

To decouple lasting effects of maternal protein restriction during pregnancy on maternal postpartum physiology versus fetal neurodevelopment, pups were cross fostered at birth to dams gestationally exposed to PR or CD to form the following groups: CD pups fostered to CD dams (CD->CD), PR pups fostered to CD dams (PR->CD), CD pups fostered to PR dams (CD->PR), PR pups fostered to PR dams (PR->PR) (FIG. 2A). Moreover, to examine effects of maternal protein undernutrition, specifically during pregnancy, on postnatal health of the offspring, PR-fed dams were switched to CD at parturition, and all pups were reared on CD from birth in order to isolate PR to the gestational period. Of all the experimental groups, PR pups fostered to PR dams exhibited the smallest litter sizes by weaning age (FIG. 1J) and the lowest total litter survival at 26.5% (FIG. 1K). CD pups fostered to PR dams exhibited intermediate reductions in litter survival to 46.7%, whereas PR pups fostered to CD dams exhibited 100% survival, as did CD pups fostered to CD dams. These results indicate that offspring survival is determined by an interaction between direct effects of gestational PR on fetal health and indirect effects of gestational PR on maternal health persisting into the postnatal period. It further indicates that gestational PR-induced alterations in maternal, but not fetal health, are sufficient to reduce offspring survival. There were no significant differences in pup weights in the first two weeks of life (FIG. 1L), suggesting that fetuses from PR dams exhibit weight recovery with postnatal rearing on CD.

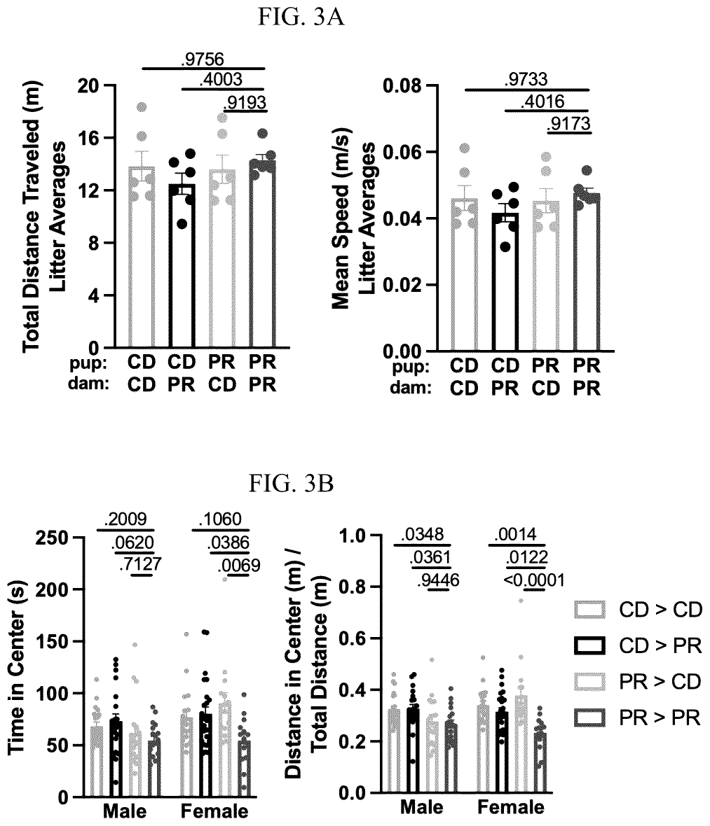

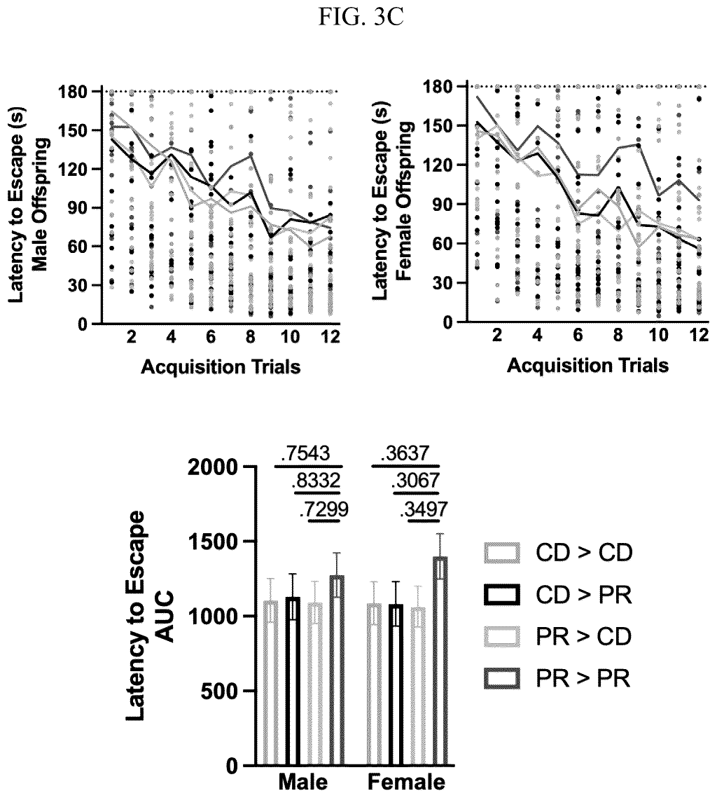

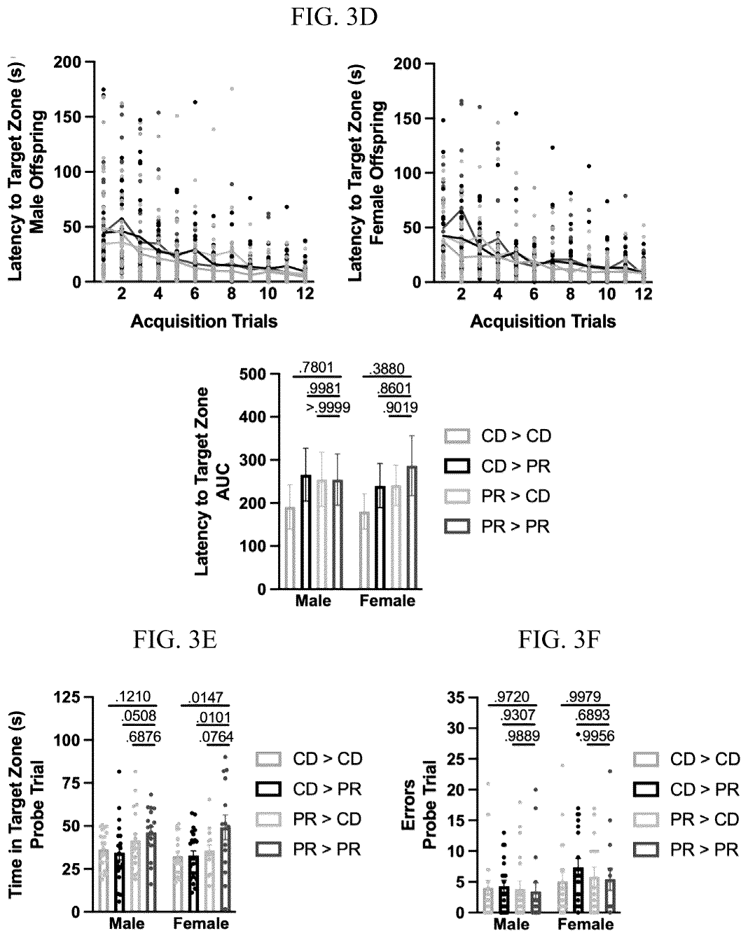

To assess lasting effects of maternal protein restriction during pregnancy on adverse neurobehavioral outcomes in the offspring, fostered offspring were weaned, reared to adulthood, and tested in behavioral assays related to anxiety and cognition (FIG. 2B). The open field assay is a benchmark test for stress-induced thigmotaxis as an endophenotype of anxiety-like behavior. Adult PR offspring previously fostered to PR dams and reared since birth on CD (PR->PR) displayed significantly reduced time and distance in the center of open field (FIG. 2C-D), as compared to other experimental groups, with no difference in average speed or total distance traveled (FIG. 3A). This anxiety-like phenotype was more striking in females than males, with postnatal influence of CD rearing appearing to be sex-discriminatory (FIG. 3B). This female-bias in anxiety-like response was similarly reported in a multi-hit pre- and postnatal adversity model. This finding suggests that adverse postnatal interactions between maternal and fetal responses to gestational PR lead to anxiety-like behavior. The Barnes maze test is a benchmark assay of spatial learning and memory, wherein mice are trained over repeated trials to identify which out of 20 holes contains an escape box (acquisition phase) and their ability to recall the spatial location of the escape is tested in a final probe trial 24 hours post-training. During the acquisition phase, adult PR->PR offspring exhibited increased latency to escape (FIG. 2E), but no significant difference in primary latency to target zone (FIG. 2F), suggesting deficient learning. During the probe phase, these PR->PR offspring displayed increased time in the target zone (FIG. 2G), but no significant difference in errors made (FIG. 2H), suggesting adequate 24-hour recall, but increased perseveration. There were no overt sex differences in cognitive deficits seen in offspring of PR-fed dams (FIG. 3C-F). These findings suggest that maternal PR during pregnancy alters both maternal physiology and fetal development in ways that interact postnatally to engender lasting behavioral impairments in offspring with adequate postnatal nutrition.

### Example 2: Maternal Protein Restriction Alters Fetal Brain Signatures

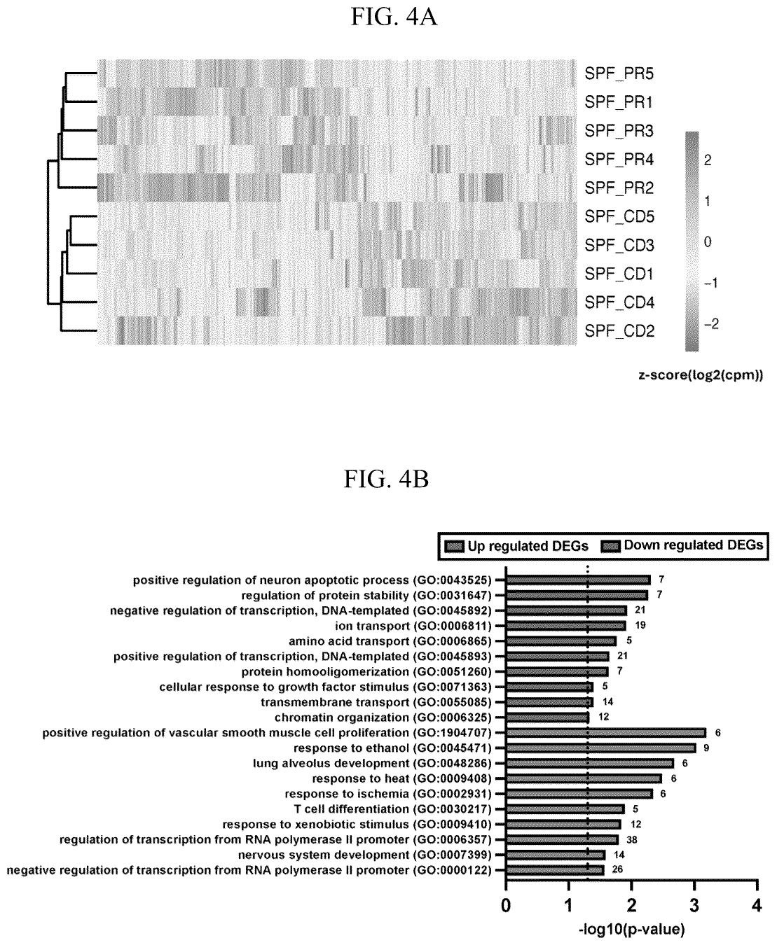

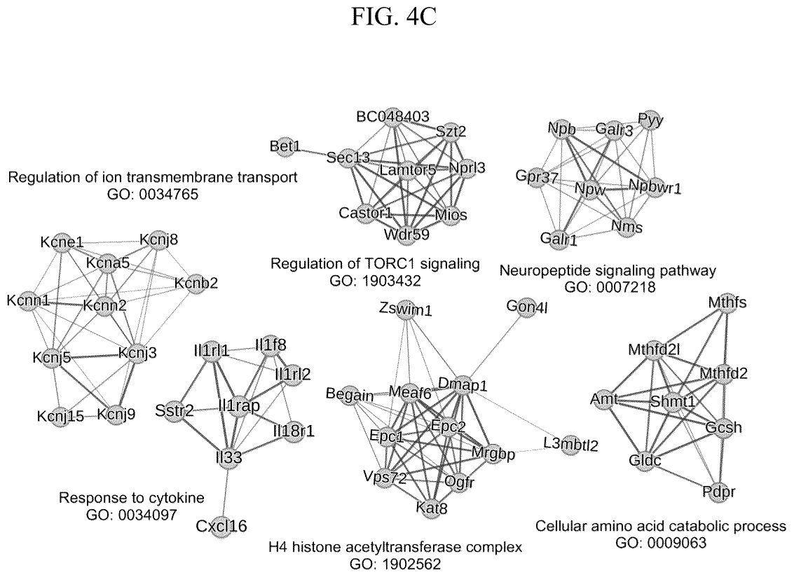

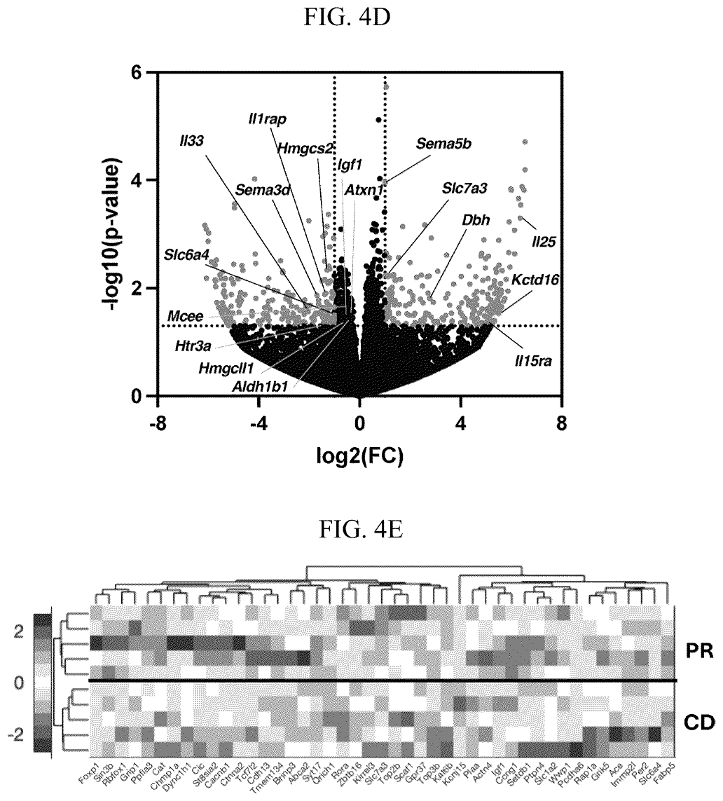

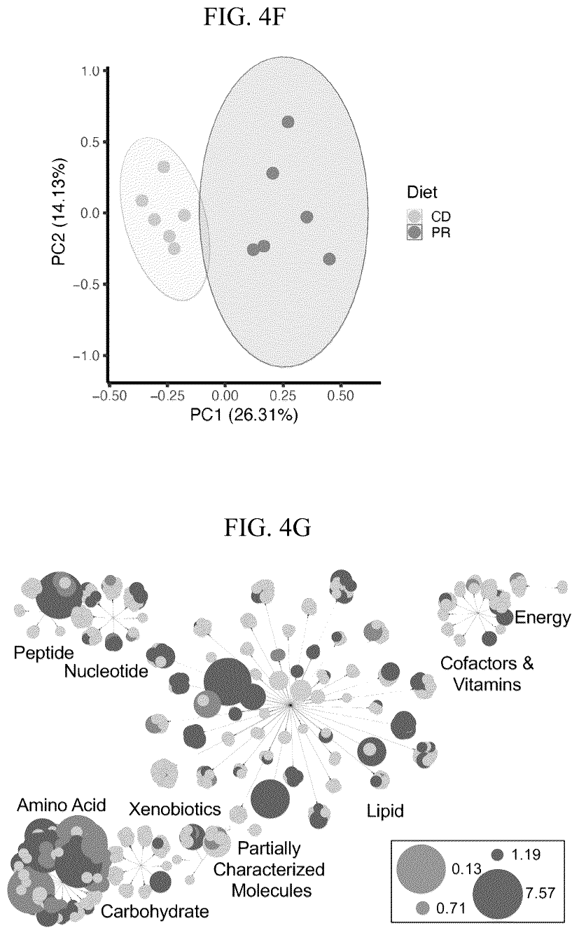

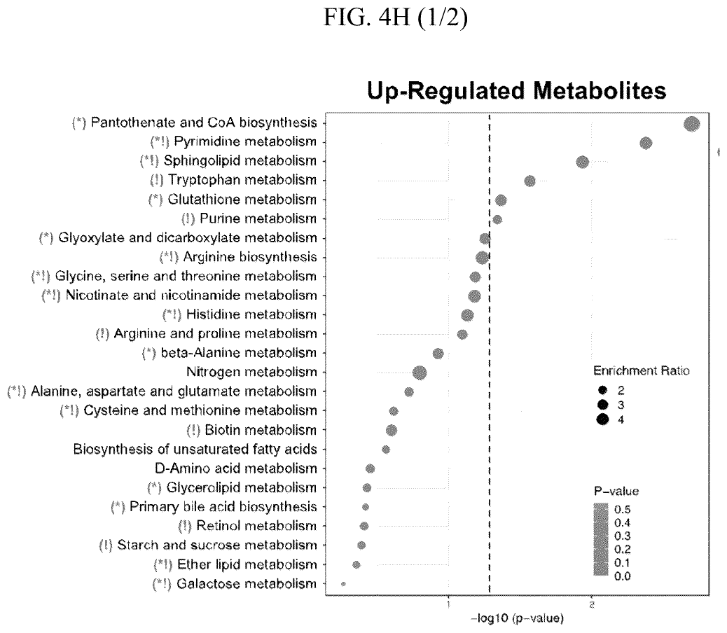

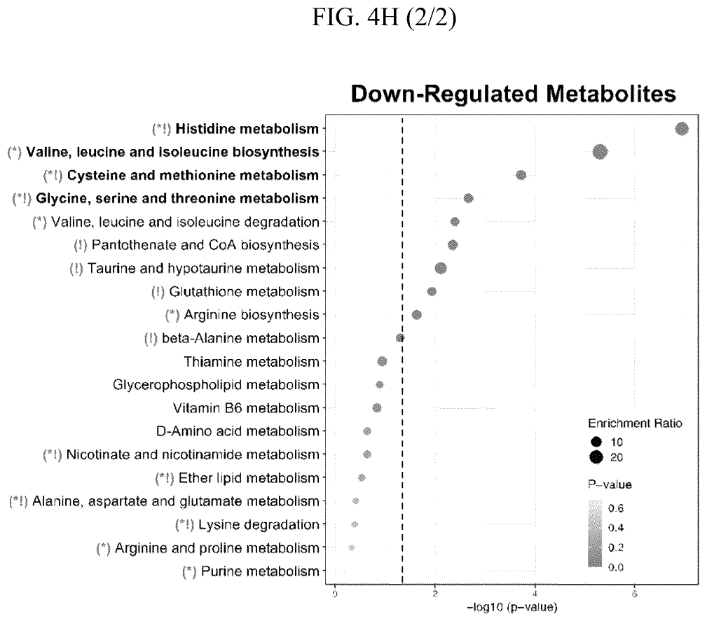

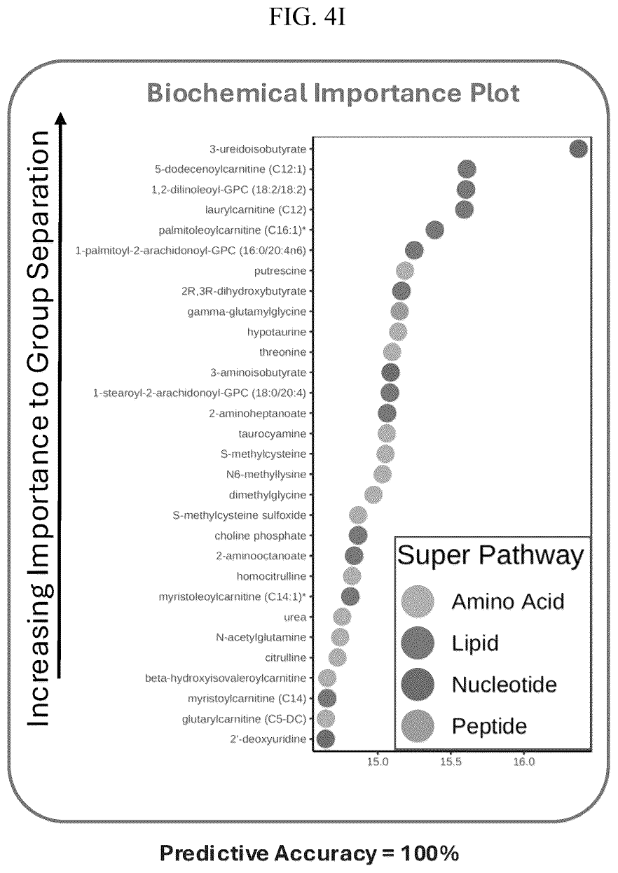

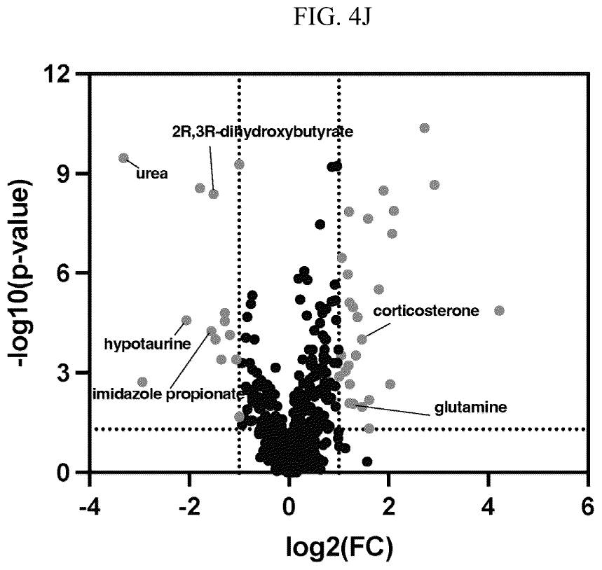

Maternal malnutrition can alter the trajectory of fetal neurodevelopment to result in long-term changes in brain function and behavior. To uncover mechanisms by which maternal protein undernutrition programs adversely affect behavioral outcomes in the offspring, the effects of maternal PR on E18.5 fetal brains by transcriptomic profiling were examined. 505 significantly up-regulated and 423 significantly down-regulated genes in response to maternal PR were identified (FIG. 4A). By gene ontology analysis, the upregulated genes related to biological processes such as neuronal apoptosis (reported to be increased in fetal brain following protein restriction), ion and amino acid transport, and response to growth factor (FIG. 4B), suggesting compensatory mechanisms related to brain sparing. Upregulated genes were mapped to protein networks for cellular amino acid catabolism, H4 histone acetyltransferase, and neuropeptide signaling (FIG. 4C). Downregulated genes related to mapped to protein networks for regulation of TORC1 signaling and ion transmembrane transport, and biological processes such as T-cell differentiation and nervous system development, potentially highlighting or neuroimmune disruptions, as previously implicated in malnourished children. Particular neurobehaviorally-relevant genes were significantly altered in fetal brains from PR dams, including downregulated serotonin transporter (Slc64a) and serotonin receptor (Htr3a), insulin-like growth factor 1 (Igf1), which modulates anxiety and stress response, and Ataxin 1 (Atxn1), related to learning and hippocampal deficits. Conversely, activity-dependent neuroprotective protein (Adnp), implicated in cognitive and social impairments and stress response, and dopamine beta hydroxylase (Dbh), relevant to learning and memory were upregulated in fetal brains from PR dams. 45 of the differentially expressed genes in PR fetal brains (FIG. 4D) aligned with risk genes for autism spectrum disorder (ASD) (FIG. 4E).

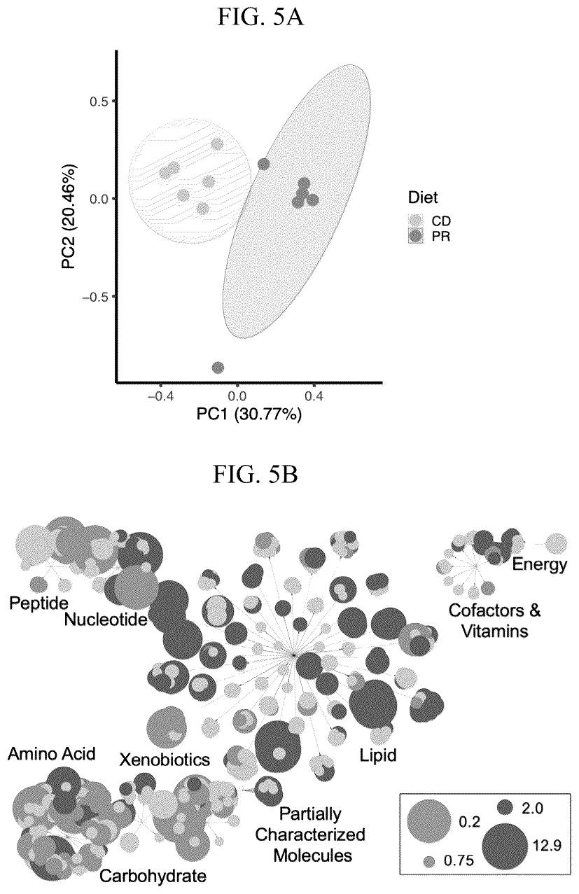

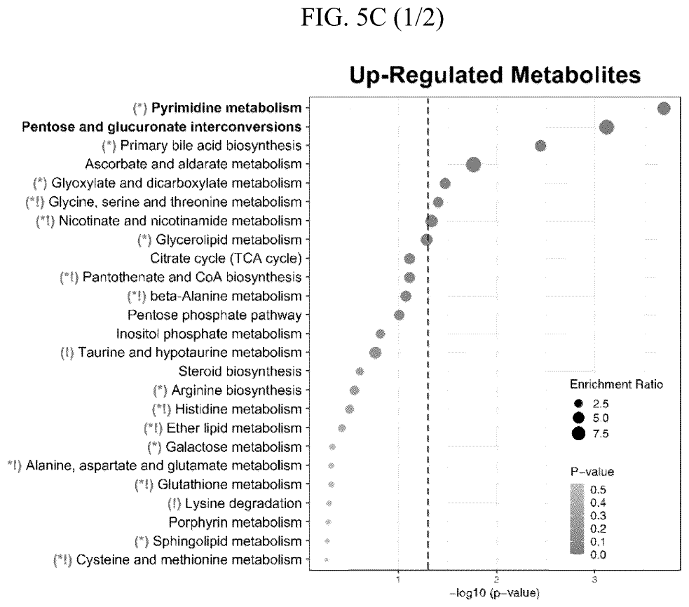

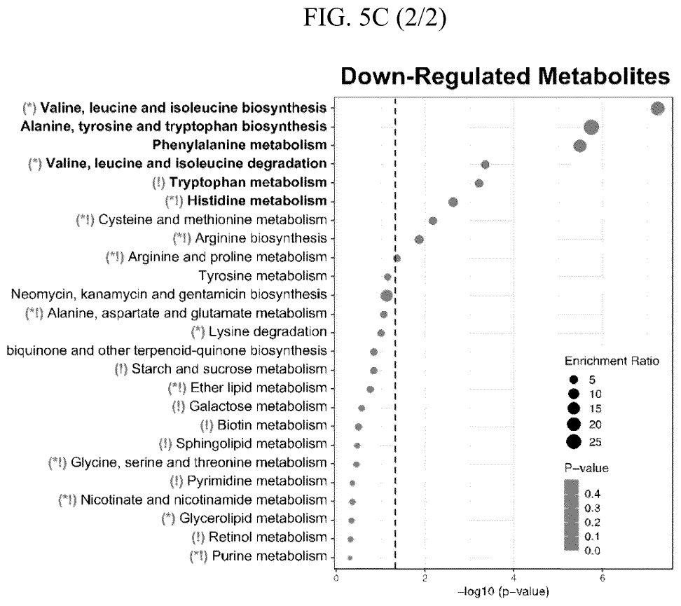

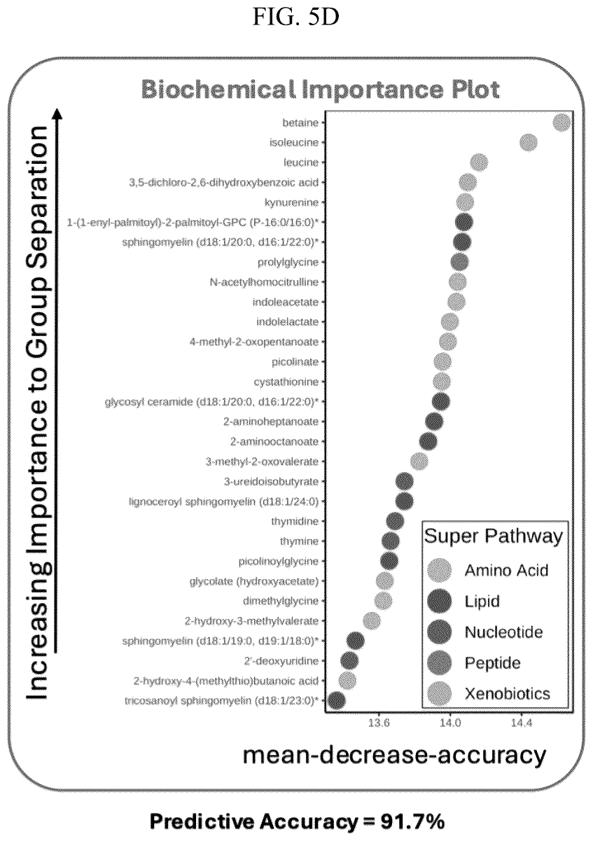

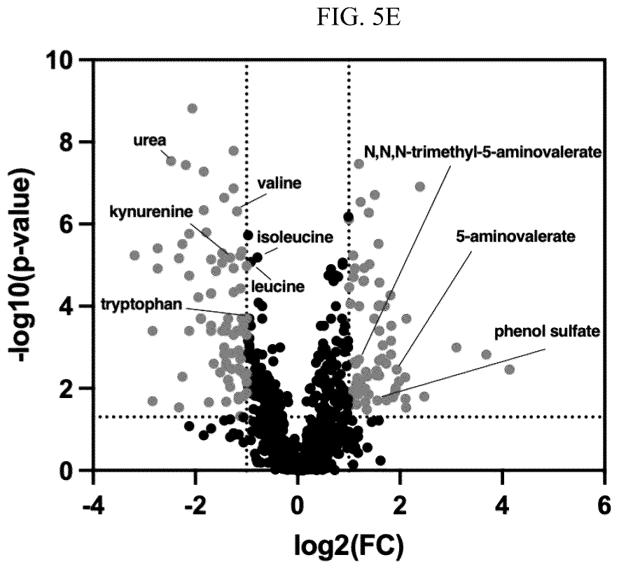

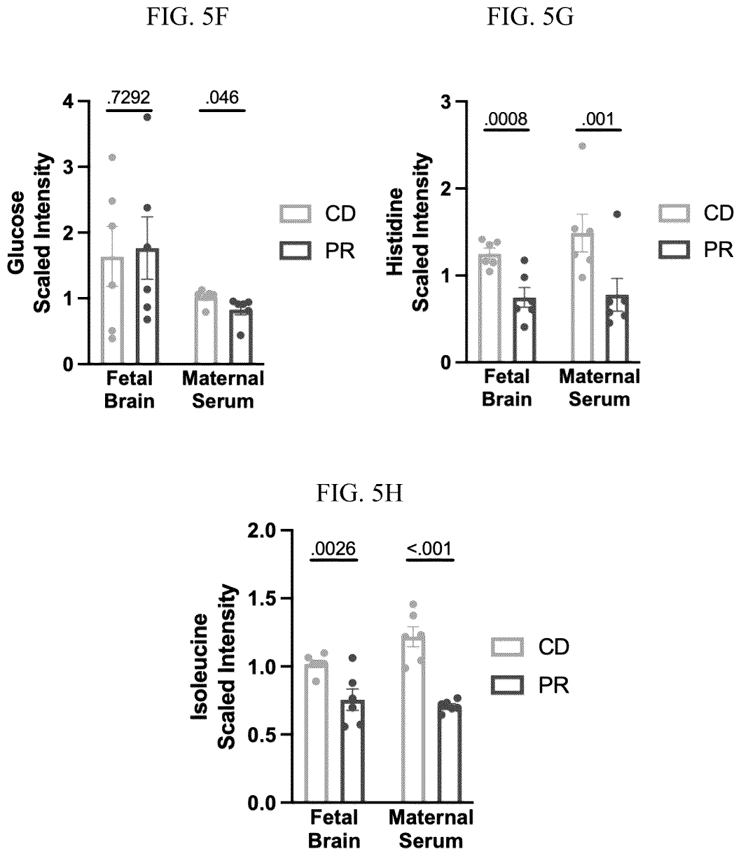

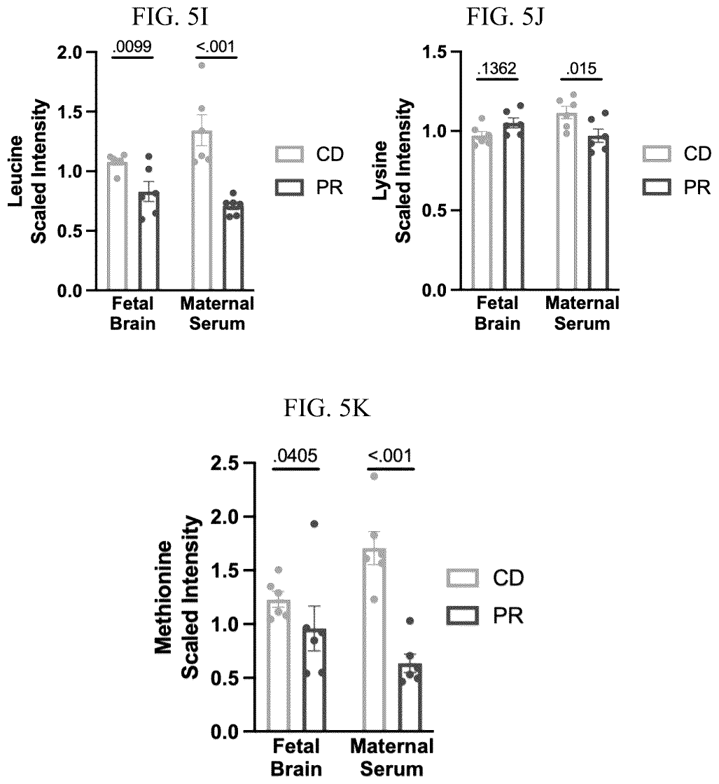

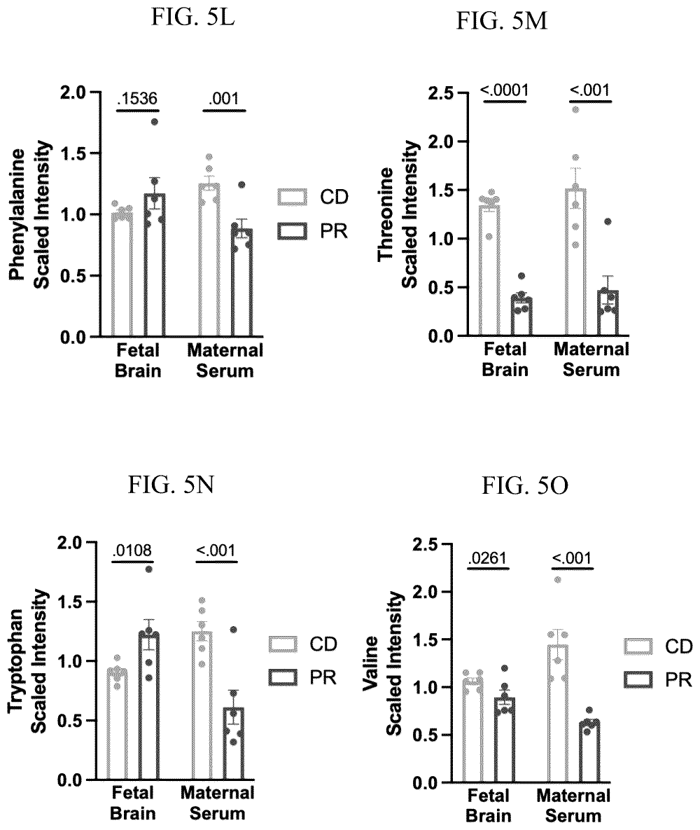

These PR-induced alterations in fetal gene expression corresponded with widespread changes in levels of fetal brain metabolites. Liquid chromatography tandem mass spectrometry-based metabolomic profiling of E18.5 brains yielded detectable levels of 681 identified compounds, spanning amino acid, peptide, carbohydrate, energy, lipid, nucleotide, cofactor and vitamin, and xenobiotic super pathways. By principal component analysis, metabolomic profiles of fetal brains from PR dams were clearly discriminated from CD controls along PC1 (FIG. 4F), with statistically significant alterations in 220 metabolites (Table 3). The most highly affected metabolite classes were amino acids (98) and lipids (84) (FIG. 4G). Of the amino acid-related metabolites that were significantly altered in fetal brains from PR dams relative to CD controls, 50% were increased (and 50% were decreased), suggesting complex regulation of amino acids. By comparison, metabolomics in maternal serum were further profiled, revealing 332 serum metabolites that were statistically significantly altered by PR consumption (FIG. 5A, Table 4). As in fetal brains, the majority were amino acids (107) and lipids (144) (FIG. 5B). However, in contrast to fetal brains, the alterations in amino acid-related metabolites seen in maternal serum were mostly reductions (81.3%, as compared to 50% in fetal brain) (Table 3 and Table 4), which is consistent with low protein intake and evidence of fetal brain sparing. In line with this, all essential amino acids and glucose showed significant reductions in PR maternal serum compared to CD (FIG. 5F-O). However, glucose, lysine, and phenylalanine showed no significant difference between PR and CD fetal brain, and tryptophan showed a significant increase in PR fetal brain compared to CD, further supporting a metabolite-specific brain-sparing phenotype. (FIG. 5F, J, L, N). Without being bound by theory, it is possible that preferential shunting of nutrients to the developing brain may come at the cost of other organ systems and contribute to PR-induced metabolic impairments.

By metabolite set enrichment analysis, fetal brain metabolites that were reduced by maternal PR mapped to 14 metabolic pathways, with histidine metabolism, glycine and serine metabolism, methionine metabolism, and homocysteine degradation persisting after statistical correction (FIG. 4H). Similar to fetal brain metabolites, maternal serum metabolites related to pyrimidine metabolism were upregulated by PR, whereas pathways related to tryptophan metabolism and valine, leucine, and isoleucine degradation were downregulated (FIG. 5C). Random forest analysis identified a top 30 metabolites in fetal brain and maternal serum that returned 100% and 91.7% predictive accuracy, respectively (FIG. 4I, FIG. 5D). The most severely depleted individual metabolites in PR fetal brains included urea (10.1% the levels of CD fetal brains), a product of protein catabolism, and hypotaurine (24.0% the levels of CD fetal brains), a precursor of taurine, which directly supports synaptic formation and transmission in the developing brain (FIG. 4J). Maternal serum also showed reduced urea, in addition to tryptophan and branched-chain amino acids (BCAA) metabolites (FIG. 5E). Conversely, PR fetal brains had a 276.9% increase in corticosterone, which is consistent with increased corticosterone in maternal serum by ELISA (FIG. 1H). Synthetic corticosteroids which mimic glucocorticoid activity during gestation have been previously reported to impair hippocampal synaptic development in primates, and myelination and brain growth in sheep. Furthermore, maternal cortisol has been correlated with sex-specific amygdala connectivity and internalizing symptoms in humans. Taken together, these results indicate that maternal protein undernutrition during pregnancy modifies transcriptional and metabolomic profiles in the late gestational fetal brain, which can contribute to fetal neurodevelopmental programming in ways that interact postnatally with maternal factors to yield behavioral abnormalities in adult offspring.

Transcriptomic and metabolomic profiling of fetal brains of PR-fed dams revealed some possibilities for how PR induces behavioral deficits, and how 10M ameliorates them. For one, tryptophan and tryptophan-related metabolic pathways were significantly downregulated in maternal serum, but significantly upregulated in fetal brain in response to gestational PR. Additionally, the Slc6a4 gene encoding the serotonin transporter and the Htr3a gene encoding a serotonin receptor are significantly decreased in PR fetal brains. Finally, a tryptophan catabolite, 3-indoxyl sulfate, was among the 10M group that normalized behavior in offspring. These findings suggest abnormal tryptophan and serotonin signaling pathways as candidate contributors to subsequent behavioral deficits in offspring. Indeed, tryptophan and serotonin were altered by protein restriction during gestation and lactation in rats, serotonin regulates many neurodevelopmental processes, and altered levels during gestation have been linked to behavioral impairments, including anxiety and cognitive, in offspring. In addition, disruptions to the hypothalamic-pituitary-adrenal axis could be at play, as elevated corticosterone in maternal serum and in fetal brains of protein-restricted dams was observed, the former of which is attenuated by 10M supplementation. Stress during critical periods can be neurotoxic, is known to interact with both maternal microbiome and fetal neurodevelopment, and to precipitate anxiety- and cognitive-related behavioral deficits in offspring. Finally, neuroimmune processes may be disrupted, as downregulated T-cell differentiation in PR fetal brains by transcriptomic pathway analysis was observed. Indeed, T-cell differentiation promoted by maternal gut bacteria has been reported to underlie behavioral deficits in offspring following maternal immune activation.

### Example 3: Murine Maternal Protein Restriction Perturbs Peripartum Physiology

Maternal behavioral and physiological care during early life has profound and persistent effects on offspring neurobehavioral development. Results from cross-fostering experiments indicate that PR-induced alterations in offspring behavior require antenatal exposure of both fetuses and mothers to PR during gestation (FIG. 2). To gain insight into how protein undernutrition during pregnancy impacts maternal health to disrupt offspring behavioral development, first, dams were tested for postpartum behavior related to maternal care. The pup retrieval test is a benchmark behavioral assay that assesses the mother's response to retrieve pups upon their removal from the nest. There were no statistically significant differences between PR and CD dams in their latency to full retrieval of their fostered pups, either PR or CD (FIG. 1M). Consistent with this, there were no differences in pup righting reflex or ultrasonic vocalizations upon maternal separation (FIG. 1N-0). These results suggest no overt difference in pup-directed maternal care across dams previously fed PR or CD during pregnancy.

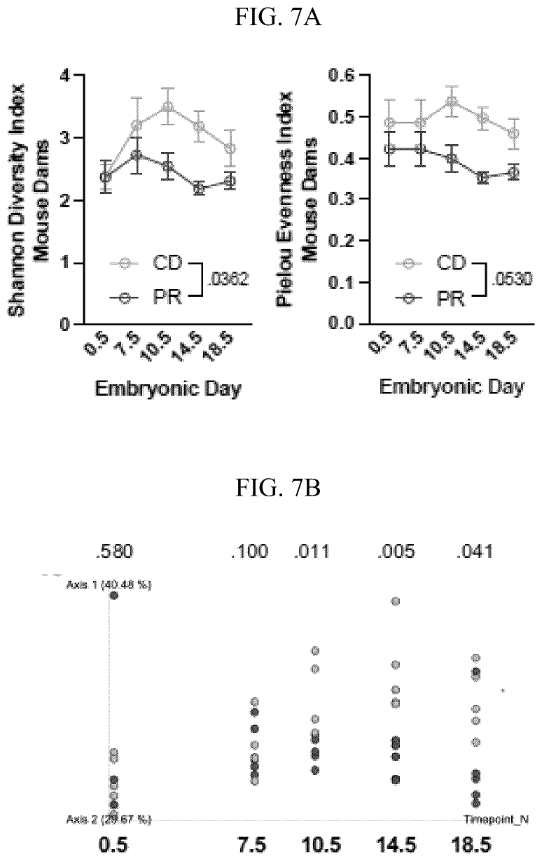

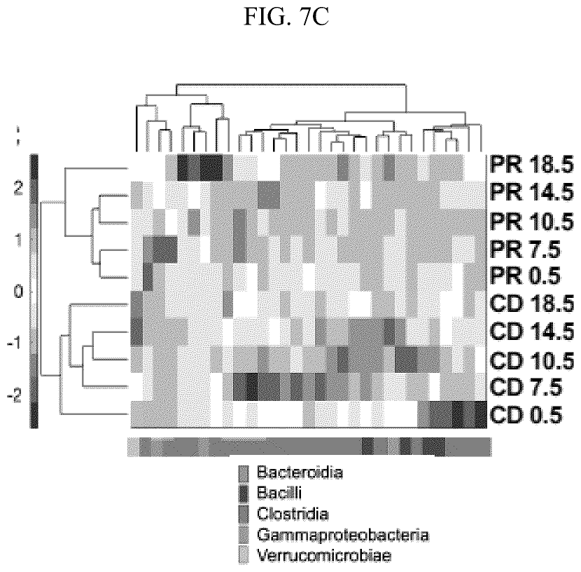

In addition to maternal care, the maternal gut microbiome is increasingly appreciated for its roles in promoting healthy neonatal development by supporting nutrition and immune development. Alterations in the maternal microbiome, observed in various models of malnutrition, can disrupt early neurodevelopment and elicit lasting changes in offspring behavior. To determine effects of protein restriction on the composition of the maternal gut microbiota, 16S rRNA gene sequencing of fecal samples collected over the course of gestation from dams fed PR or CD was performed. Consistent with previous reports, bacterial alpha-diversity increased with pregnancy in CD-fed dams (FIG. 7A). However, this was prevented by PR, as fecal microbiota from PR dams displayed significantly decreased Shannon diversity [a measure of both richness (the number of different species) and evenness (the amount within each species)] and Pielou's evenness as compared to CD controls (FIG. 7A). Principal coordinates analysis of weighed Unifrac distances discriminates microbiota from PR dams away from CD controls by E10.5 (FIG. 7B). Differences in beta diversity were driven by statistically significant alterations in the relative abundances of 31 bacterial taxa (FIG. 7C), which were dominated by Clostridia species. Reductions align with the finding that Clostridia are the main metabolizers of dietary protein and amino acids. Altogether, these results indicate that maternal protein undernutrition during pregnancy reduces diversity of the maternal microbiota, which corresponds with alterations in functional profiles in the fetal brain and neurobehavioral deficits in adult offspring despite being reared on a standard protein diet since birth.

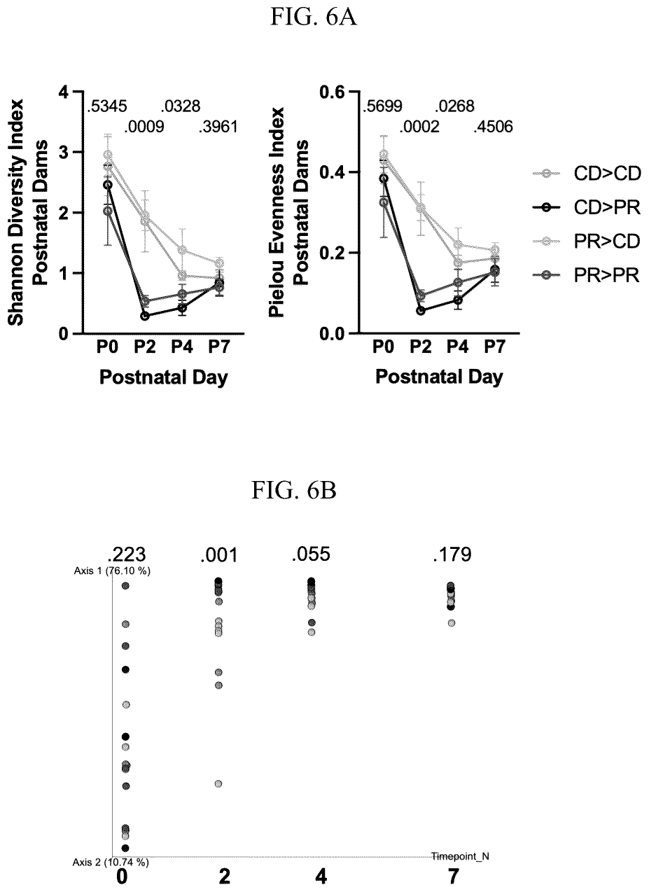

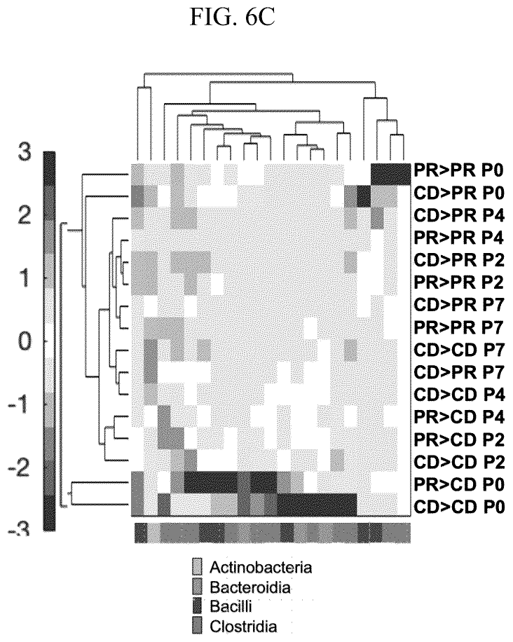

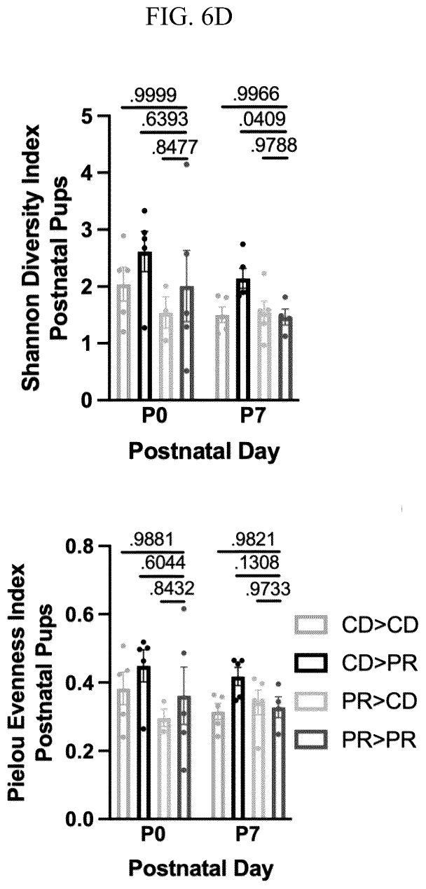

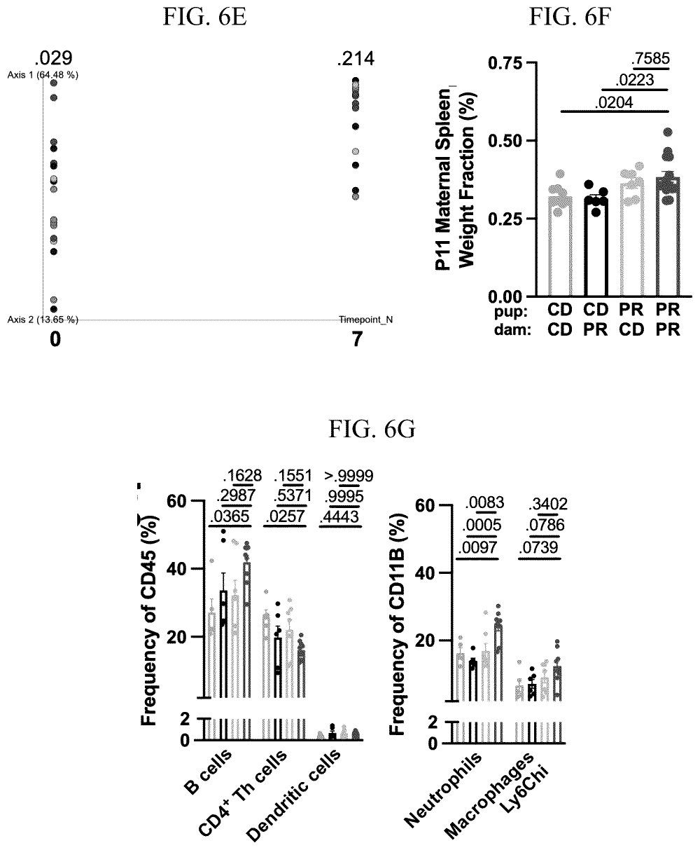

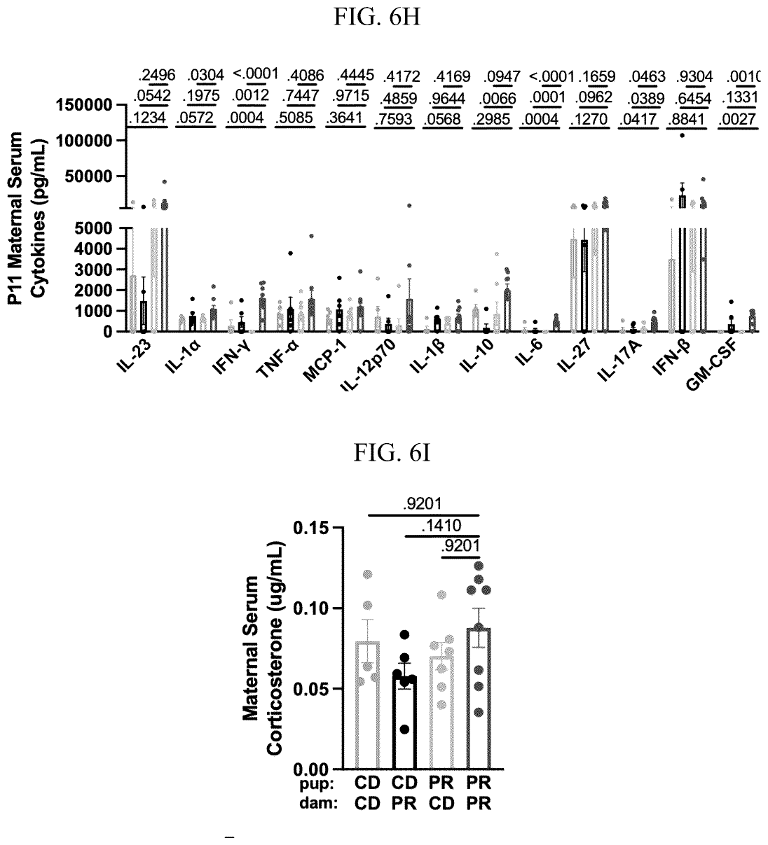

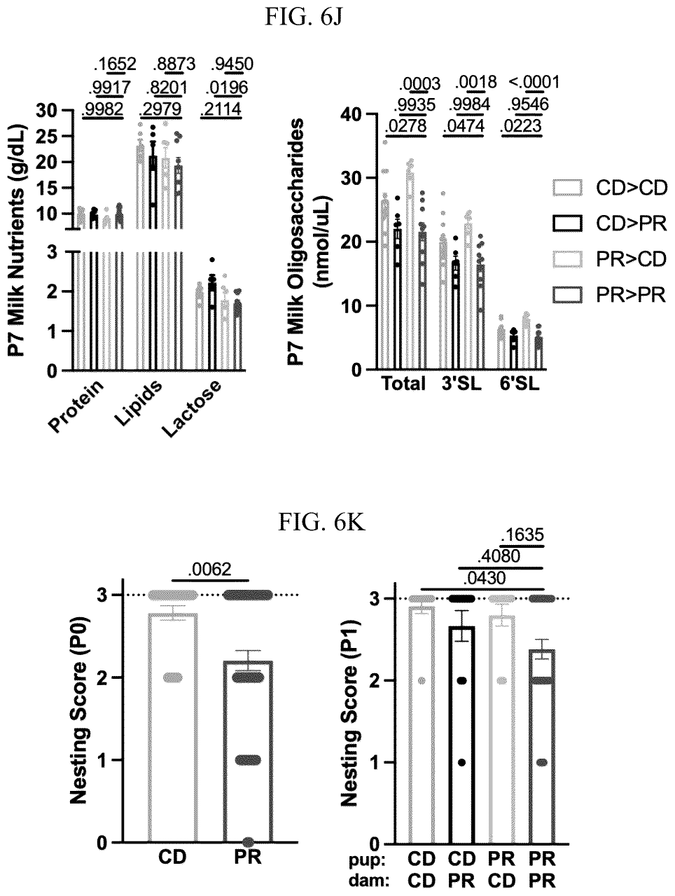

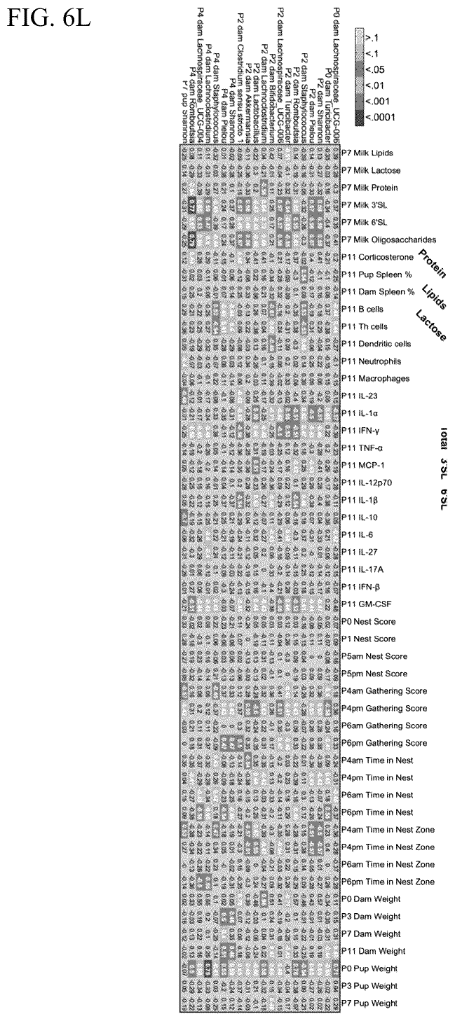

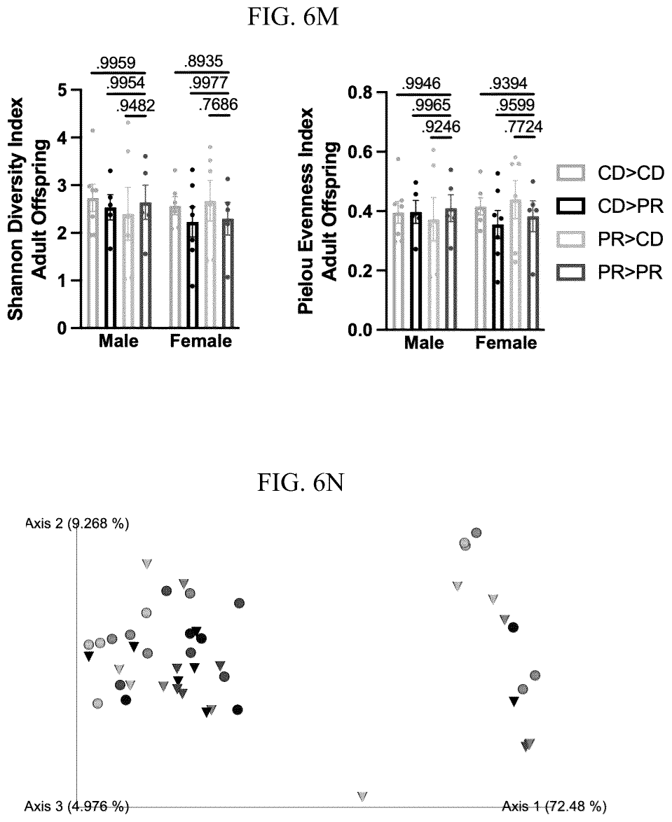

To determine whether these alterations are corrected by switching to CD on P0, dams were profiled during the postpartum period. All groups exhibited reductions in bacterial alpha-diversity following parturition (FIG. 6A), but PR dams displayed more severe reductions in Shannon diversity and Pielou evenness compared to CD dams, regardless of foster pup condition. This was seen on P2 and P4, but not P7, suggesting normalization of the microbiota after one week of consuming CD. Similarly, alterations in beta-diversity were significant at P2, near-significant at P4, and resolved by P7 (FIG. 6B). Differences were driven by 21 taxa (FIG. 6C), which were, similarly to the gestational findings, dominated by Clostridia species. 11 of these taxa, 7 of which were Clostridia, were shared between the pre- and postpartum periods. When analyzed by individual day, differences resolved by P7. These results indicate that PR reduces diversity of the maternal microbiota during pregnancy and the early postpartum period, persisting between 4-7 days after switching to CD. These reductions correspond with alterations in functional profiles in PR fetal brain and neurobehavioral deficits in adult offspring.

To determine whether maternal PR during pregnancy leads to postnatal alterations in microbiota of cross-fostered offspring, pups were profiled during the early postnatal period and adult offspring at the time of behavioral testing. Pups showed no differences in alpha-diversity at P0, but at P7, PR->PR pups had lower Shannon diversity compared to CD->PR pups (FIG. 6D). Pups showed differential beta-diversity based on gestational diet at P0 only (FIG. 6E) but no particular genera survived statistical correction. Adult offspring exhibited no differences across condition or sex (FIG. 6M-N). These findings suggest that diet has a lingering effect on the maternal microbiome that resolves within a week of giving birth, and cross-fostered pups show transient disruptions that resolve prior to adulthood.

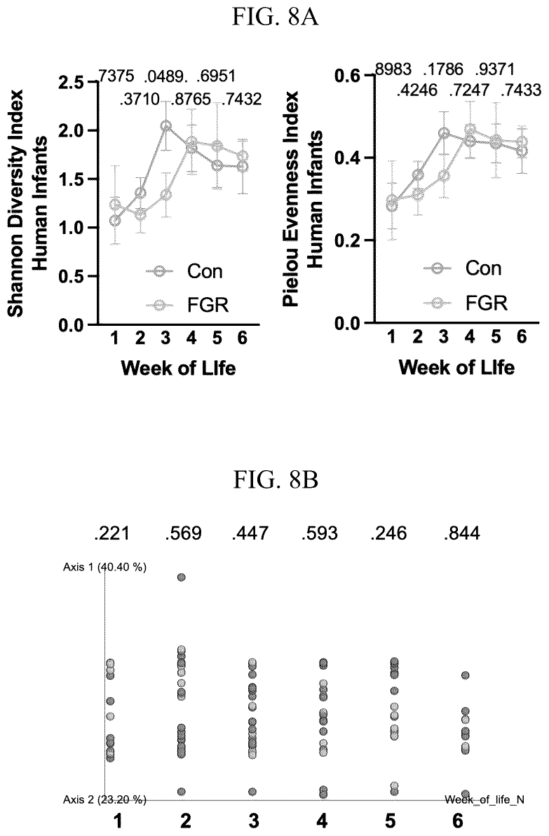

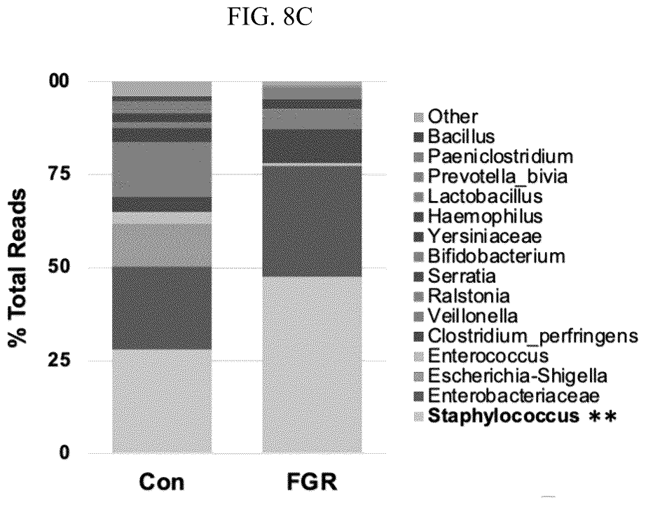

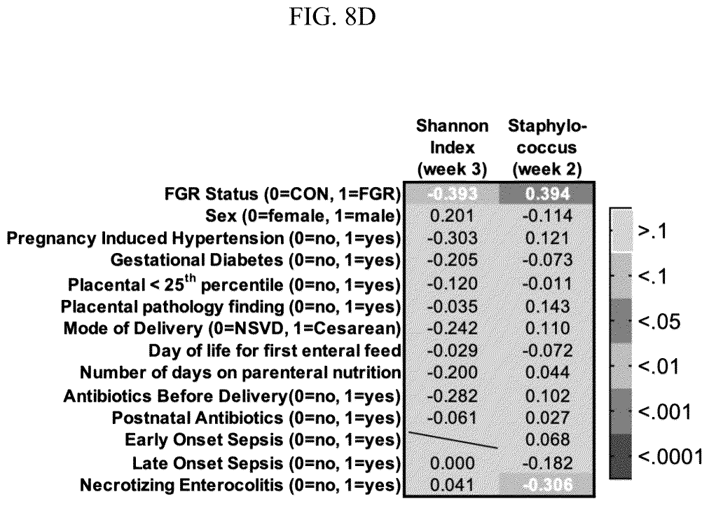

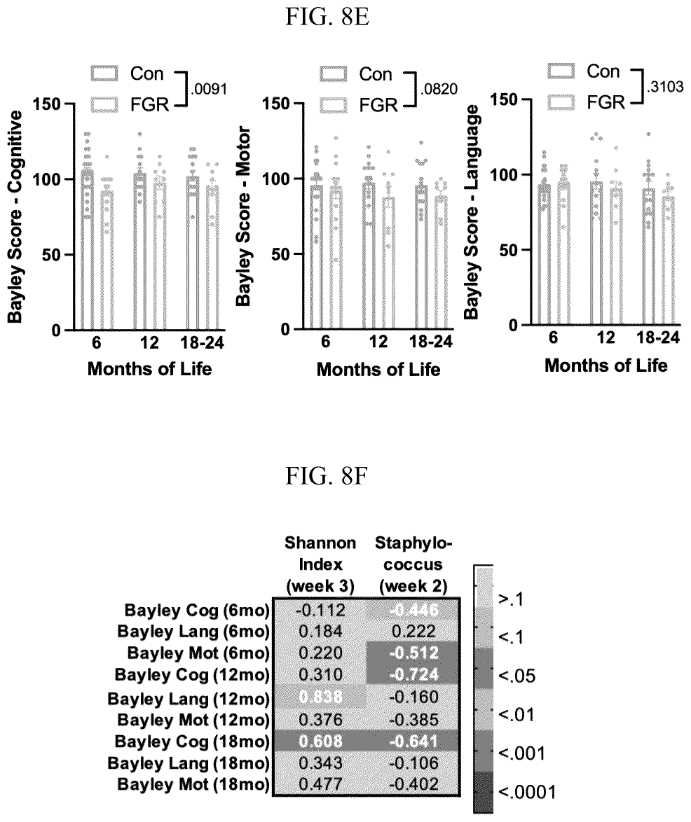

Microorganisms from the maternal microbiota seed the infant gut microbiota at birth, serving as initial colonizers that inform infant metabolic and immune development. In the maternal protein restriction mouse model for FGR, alterations in the maternal microbiota precede the development of neurobehavioral abnormalities in adult offspring. To gain insight into whether similar relationships are seen in a related human condition, associations between the early life microbiome and neurocognitive outcomes in individuals from a cohort of preterm infants with or without FGR were examined. Compared to 37 non-FGR preterm controls, 16 FGR preterm infants exhibited decreased alpha diversity of the fecal microbiota, with reduced Shannon diversity at week 3 of life, but no difference in subsequent weeks (FIG. 8A), suggesting delayed maturation of the gut microbiome. Taxonomic data did not show visible clustering of FGR vs non-FGR samples by principal coordinates analysis (FIG. 8B), indicating no widespread alterations in beta diversity. However, FGR samples exhibited significant alterations in select bacterial taxa, with increased Staphylococcus at 2 weeks of life (FIG. 8C). These alterations in microbiota were not attributable to statistically significant differences in infant sex, pregnancy induced hypertension, mode of delivery, day of life for first enteral feed, number of days on parenteral nutrition, antibiotics before or after delivery, early or late onset sepsis, or necrotizing enterocolitis (the latter showed a negative trending correlation with Staphylococcus) (FIG. 8D). When 28 non-FGR preterm infants were compared to 15 FGR preterm infants in a follow-up across 24 months corrected gestational age, the FGR subset scored significantly lower in the cognitive composite score on the Bayley Scales of Infant and Toddler Development III (BSID III), which is used to assess and diagnose developmental delays (FIG. 8E). Compared to non-FGR preterm controls, FGR preterm infants exhibited no statistical difference in language composite scores and a trending decrease in motor composite scores (FIG. 8E). In the subset of participants with both microbiota and BSID III outcome measures (15 non-FGR and 10 FGR), Shannon diversity at 3 weeks of life correlated positively with the language composite score at 12 months corrected gestational age and the cognitive composite score at 18-24 months corrected gestational age, while Staphylococcus at 2 weeks of life correlated negatively with cognitive composite score at 6, 12, and 18-24 months corrected gestational age, and trended towards a negative correlation with the motor composite score at 6 months corrected gestational age (FIG. 8F). Staphylococcus-dominated microbiota in preterm infants has previously been correlated to developmental delays and poor health outcomes. Overall, these results reveal associations between human FGR, reduced alpha diversity of the early life gut microbiota, and reduced scores in infant neurodevelopmental measures, particularly in the cognitive domain. Despite the heterogenous nature and limited sample size of this human FGR cohort, and the difference in sampling timepoint, these findings mirror our observations from the maternal protein restriction mouse model for FGR.

Measures of maternal stress, immune function, and milk nutritional content in the postpartum period, as aspects of maternal health that have substantial impacts on offspring development were assessed. There was no difference in maternal corticosterone at P11 (FIG. 6I), suggesting that the stress effect seen at E18.5 (FIG. 1H) resolves after switching to CD. In contrast to the decreased spleen size observed at E18.5 (FIG. 1E), PR dams raising PR pups had significantly larger spleens compared to both CD dams raising CD pups, and PR dams raising CD pups, but no difference when compared to CD dams raising PR pups, suggesting a pup-directed effect (FIG. 6F). PR dams raising PR pups showed immune disruption at P11, with increased splenic neutrophils (FIG. 6G), consistent with observations from E18.5 (FIG. 1F). P11 PR dams additionally displayed increased B cells and decreased CD4+ T cells compared to CD dams raising CD pups, and near-significant increased macrophages (FIG. 6F). Additionally, PR dams raising PR pups had elevated serum cytokines at P11, including IFN-γ, IL-6, IL-17A, GM-CSF, IL-1α, and IL-10 (FIG. 6H). These findings suggest an inflammatory state specifically in PR dams raising PR pups. This stands in contrast to the very limited immune phenotype observed at E18.5 (FIG. 1F-G), suggesting a delayed immune activation, known to increase risk for neurodevelopmental disorders, which is impacted by both maternal gestational diet and the gestational dietary condition of the fostered pups.

Beyond gestation, nutrition through maternal milk sets the trajectory for neurodevelopment and later life function. To determine whether gestational PR leads to lasting changes in maternal milk during the postpartum period, macronutrients were profiled in milk from P7 dams. There were no differences in proteins or lipids, but lactose was reduced in PR dams raising PR pups compared to PR dams raising CD pups, suggesting a pup-directed effect (FIG. 6J). Additionally, PR dams raising PR pups had lower levels of milk oligosaccharides, including 3′sialyllactose (3′SL) and 6′SL, compared to CD dams raising CD or PR pups, suggesting a persistent gestational diet effect (FIG. 6J). These findings suggest that despite recovery of dietary protein intake by switching to CD at birth, PR->PR offspring experience persistent malnutrition of select nutrients from maternal milk, which may compound the adverse effects of gestational PR on neurodevelopment.

To investigate relationships between the microbiota and other early postnatal influencers of neurodevelopment, all significant microbial metrics from P0-P7 in dams or pups were correlated to all other postnatal metrics (FIG. 6L). Microbial metrics correlated with milk oligosaccharides, select cytokines, and pup birth weight, suggesting an intimate relationship between the early postnatal microbiome and other physiological systems, all of which may collaborate to influence neurodevelopment of offspring.

### Example 4: Maternal Microbiome Depletion Modifies Protein Restriction

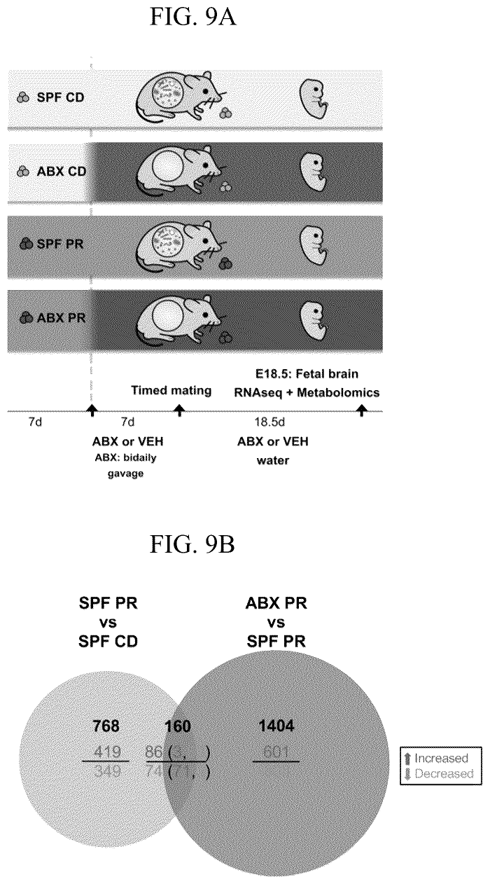

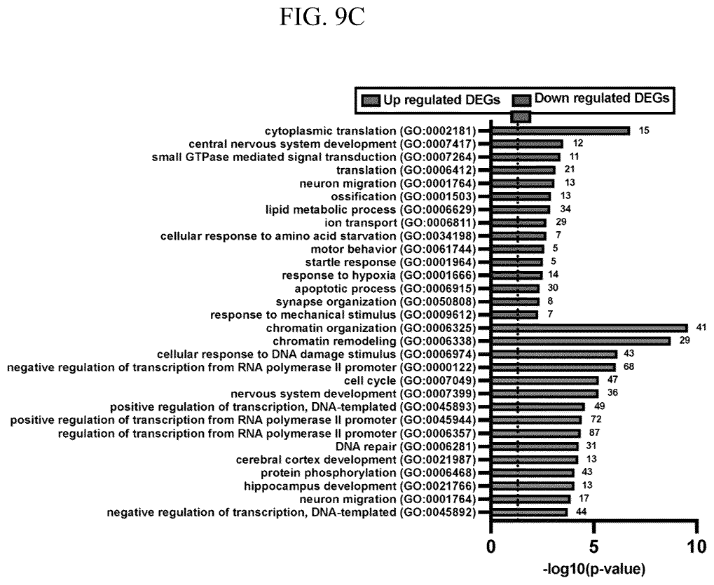

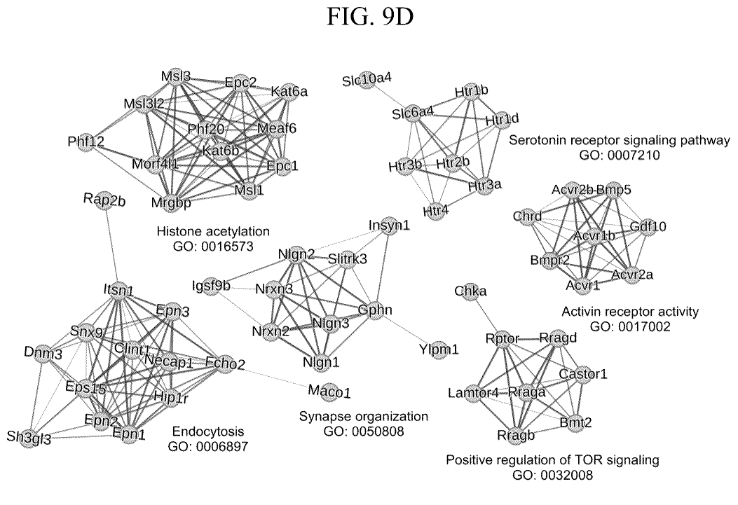

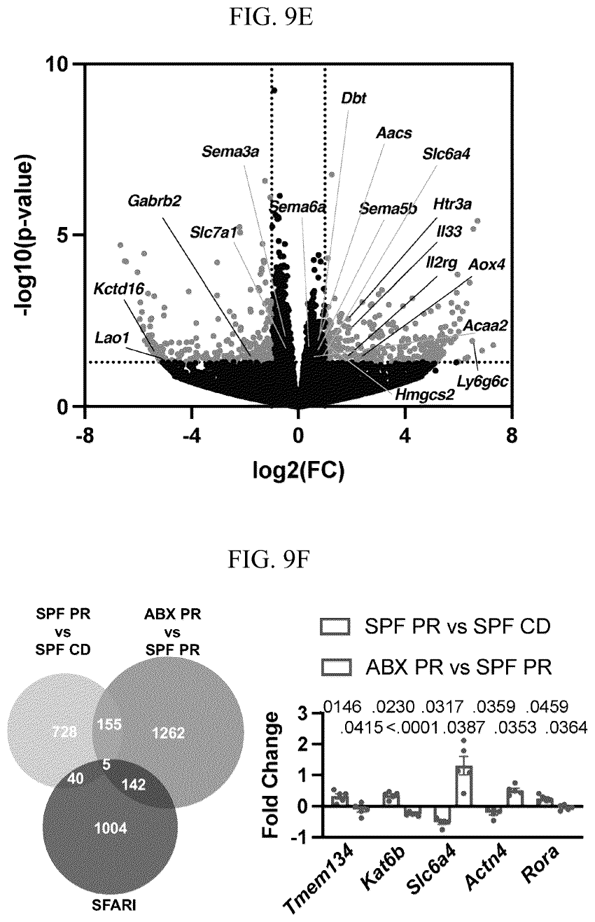

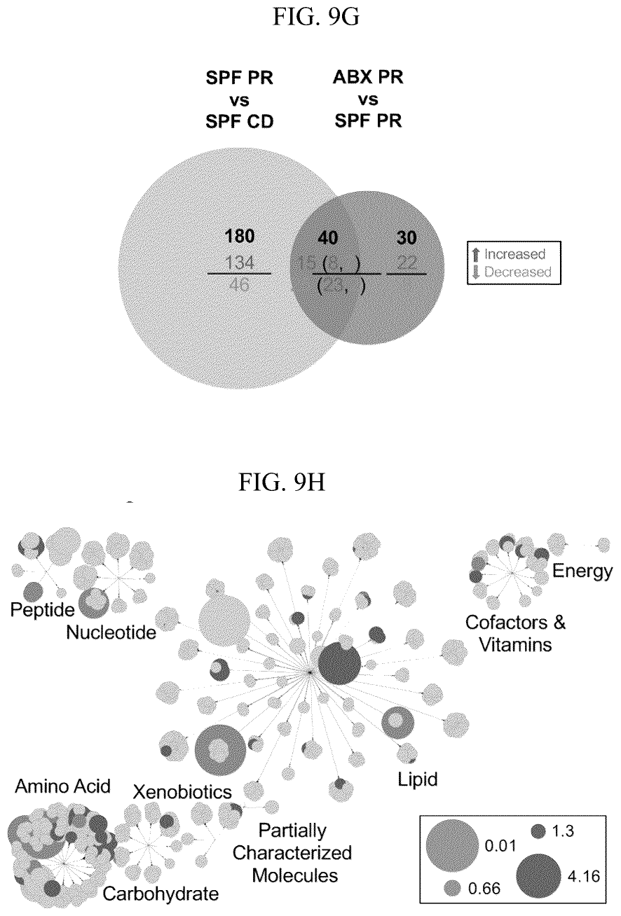

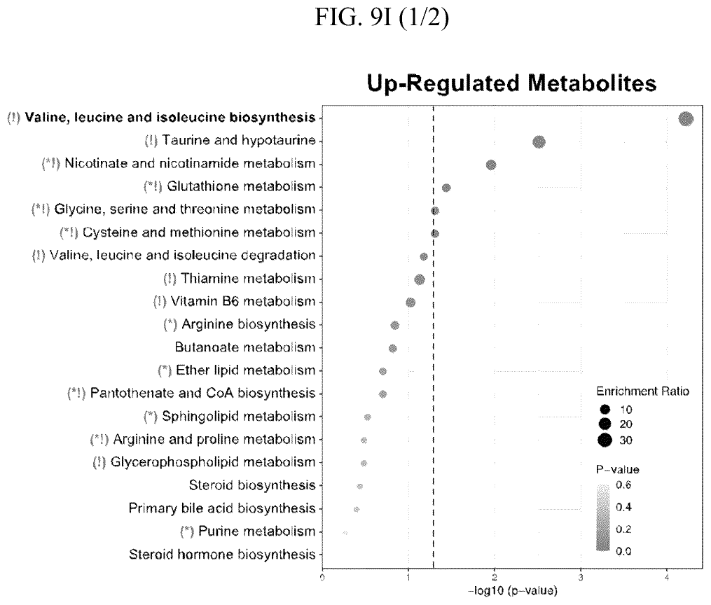

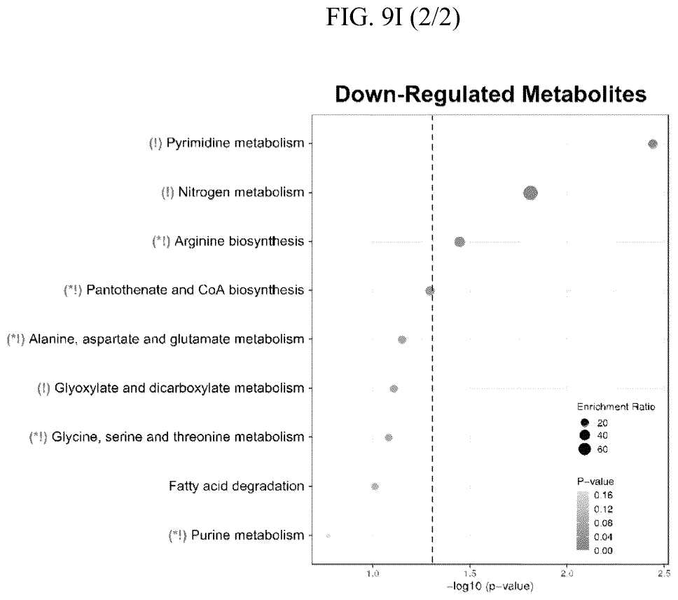

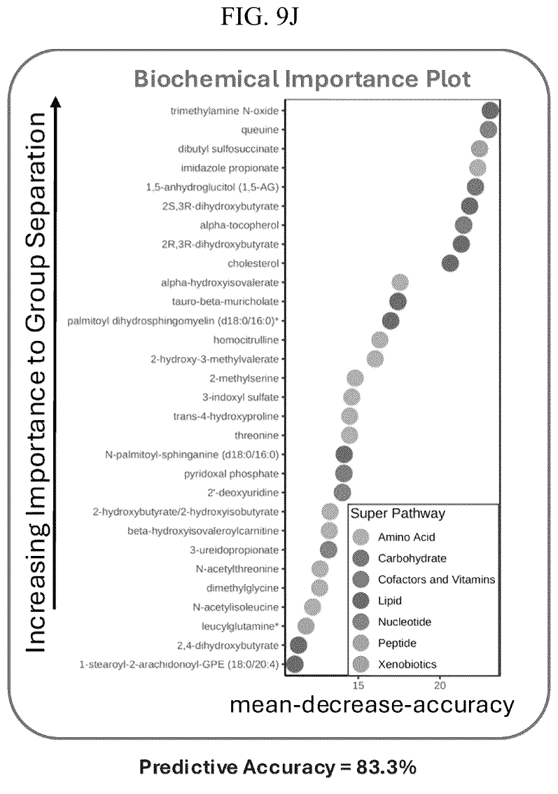

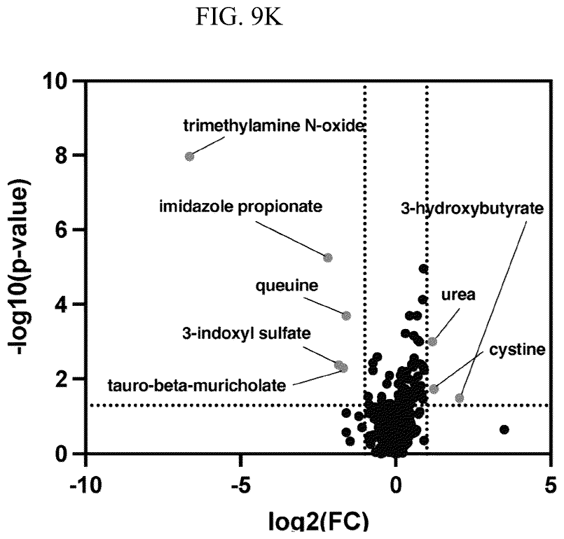

The maternal gut microbiome guides normal fetal neurodevelopment and modifies effects of maternal environmental exposures, such as immune activation, antidepressant treatment, and prenatal stress on the fetal brain. To examine influences of the maternal gut microbiome on fetal neurodevelopmental responses to maternal protein undernutrition, functional signatures in the fetal brain after depletion of the maternal microbiome in PR-fed dams were examined. Dams were treated with a cocktail of broad-spectrum antibiotics (ABX) by oral gavage twice daily for one week before breeding, and subsequently maintained on ABX in water throughout gestation (FIG. 9A). RNA sequencing revealed 1564 genes that were differentially expressed in E18.5 fetal brains from ABX-treated dams fed PR compared to conventional PR controls (specific pathogen free, SPF PR) (FIG. 9B), suggesting widespread fetal brain responses to depletion of the maternal microbiome. Of these, only 160 (10.2%) of the genes differentially expressed by maternal ABX treatment were similarly dysregulated by maternal PR relative to CD controls (FIG. 9B). By gene ontology analysis, fetal brain genes that were upregulated by ABX and PR mapped to pathways related to central nervous system development, neuron migration, synapse organization, and cellular response to amino acid starvation, whereas downregulated genes mapped to pathways including cerebral cortex and hippocampal development, as well as neuron migration (FIG. 9C). The minor overlap between the ABX and PR sets of differentially expressed genes suggests that the maternal microbiome has widespread influence on fetal brain gene expression that modifies responses to maternal protein restriction, but is unlikely to mediate adverse effects of maternal protein restriction.

In addition to modifying the fetal brain transcriptome, depleting the maternal microbiome in protein undernourished dams altered fetal brain metabolomic profiles. E18.5 brains from fetuses of antibiotic-treated PR dams exhibited significant alterations in 70 metabolites relative to SPF PR controls (FIG. 9G, Table 3). Of these, 40 metabolites overlapped with those altered by maternal PR relative to CD controls, reflecting 18.2% of metabolites altered by PR relative to CD (FIG. 9G). Metabolites altered by ABX were primarily amino acids (47.1%) and lipids (28.6%) (FIG. 9H), with significantly reduced metabolites mapping to 6 pathways, including valine, leucine, and isoleucine biosynthesis, and taurine and hypotaurine metabolism (FIG. 9I). Random forest analysis identified a top 30 metabolites with 83.3% predictive accuracy (FIG. 9J). Metabolites regulated in the same direction by both PR and ABX included imidazole propionate and 3-indoxyl sulfate, while urea was significantly increased by ABX, but significantly decreased by PR alone (FIG. 9K). Maternal ABX-induced reductions in fetal brain metabolites related to glutamate metabolism, and glycine and serine metabolism (FIG. 9I), were similarly seen with maternal PR relative to CD (FIG. 4H), suggesting an exacerbating effect of maternal microbiome deficiency on these PR-induced alterations in the fetal brain. This finding is in line with work suggesting a role for the gut microbiome in supplying the host with essential amino acids. Metabolites that were significantly elevated by depletion of the maternal microbiome mapped to 7 pathways, including taurine and hypotaurine metabolism and homocysteine degradation (FIG. 9I). Taken together, these results suggest that the diversity of the maternal microbiome alters the fetal brain in ways that modify effects of maternal protein undernutrition on offspring neurodevelopment

### Example 5: Microbiome-Based Interventions Influence Protein Restriction

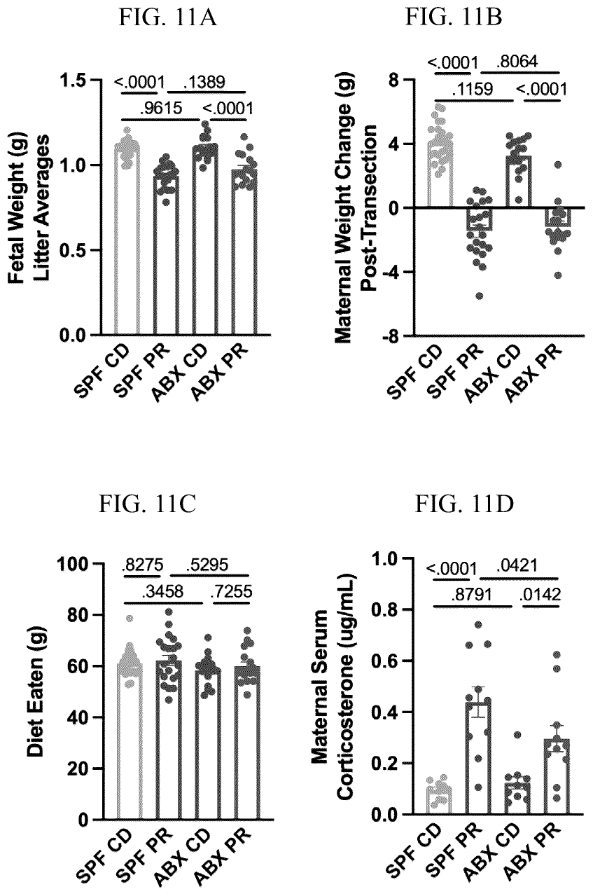

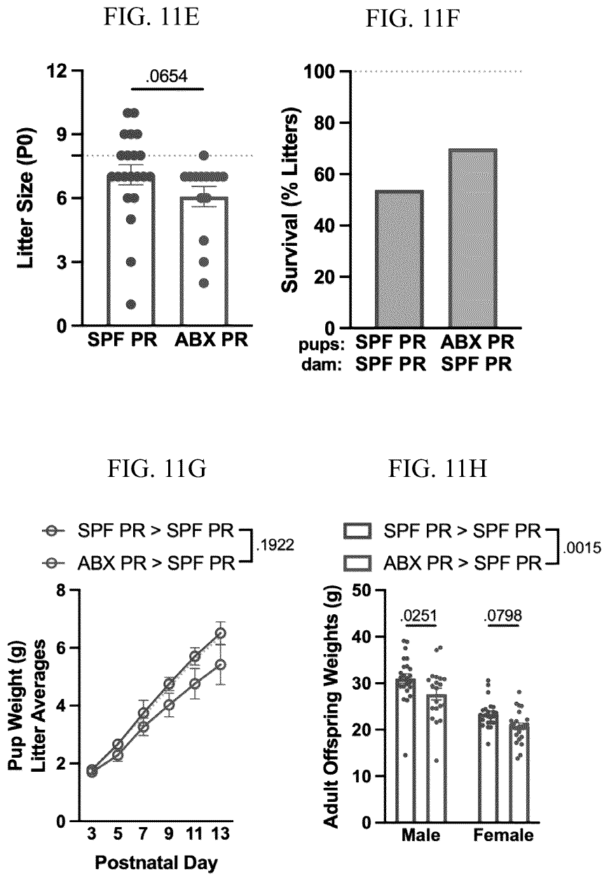

Alterations in the maternal microbiome during pregnancy are increasingly associated with impaired fetal and postnatal development of the offspring, raising the question of whether manipulating the maternal microbiome during pregnancy can change offspring health trajectories. It was found that maternal protein restriction alters maternal health status, including reductions in diversity of the maternal microbiota, which interacts with fetal development to yield abnormal behavior in adult offspring. Following the observation that ABX microbial depletion modifies PR-indued fetal brain signatures, causal effects of the maternal microbiome during pregnancy on offspring developmental responses to maternal protein restriction were assessed. To this end, growth and behavior of offspring reared from ABX-treated and PR-fed dams, fed CD at birth, and cross-fostered to untreated SPF dams fed PR during pregnancy was investigated (FIG. 10A). This experimental design isolates the effect of maternal microbial depletion and PR to the pregnancy period, and evaluates their influences on offspring developmental programming. There was no significant effect of maternal microbial depletion on PR-induced reductions in fetal size, maternal gestational weight loss, or maternal diet consumption (FIG. 11A-C). However, there was a partial attenuation of PR-induced elevation of maternal serum corticosterone levels at E18.5 (FIG. 11D). This corresponded with a trending decrease in litter size (FIG. 11E), despite overall increases in rates of litter survival at weaning (FIG. 11F). Offspring of microbiota-depleted dams fed PR during pregnancy exhibited gradual sub-significant reductions in postnatal weight over the first two weeks of life (FIG. 11G), with statistically significant decreases in weight by adulthood despite being fed CD since birth (FIG. 11H).

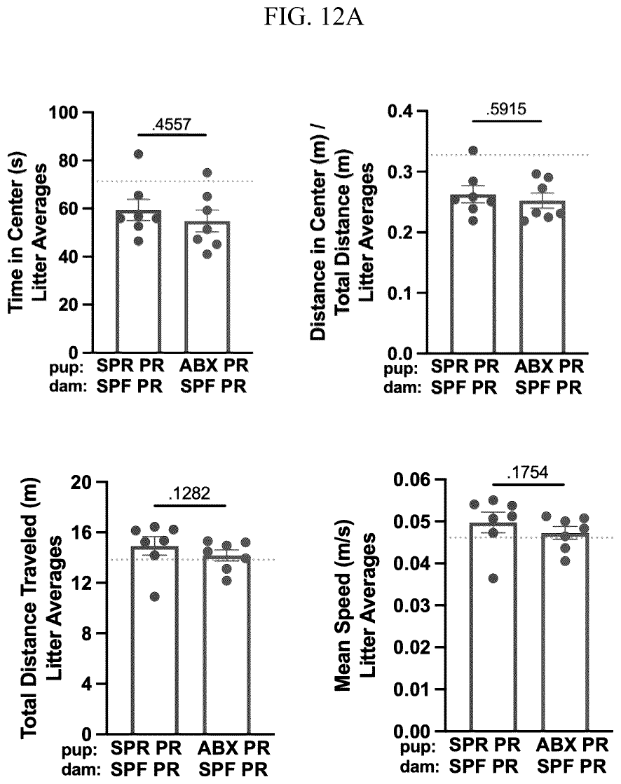

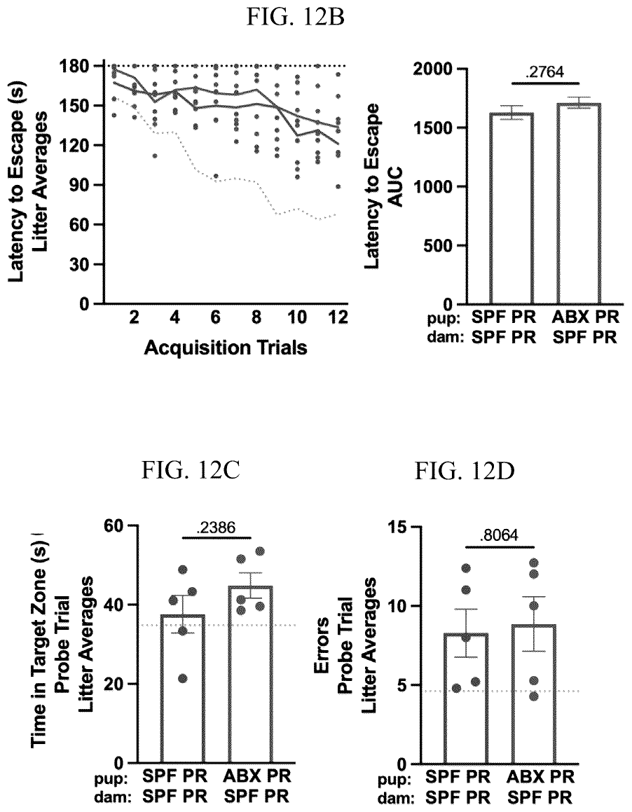

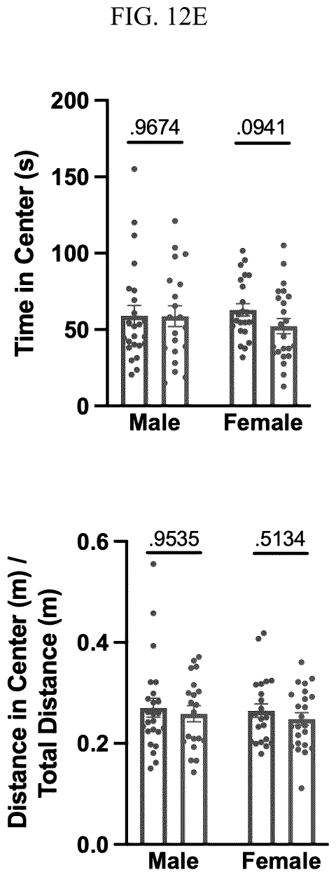

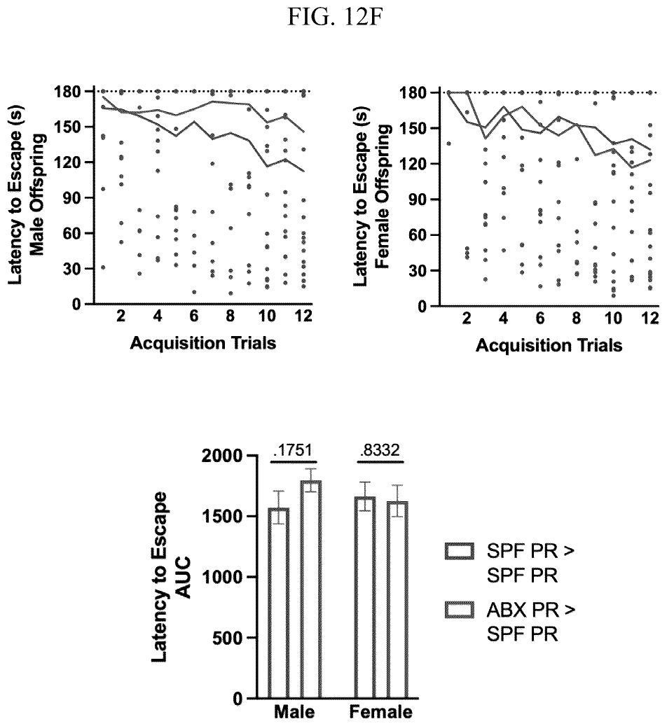

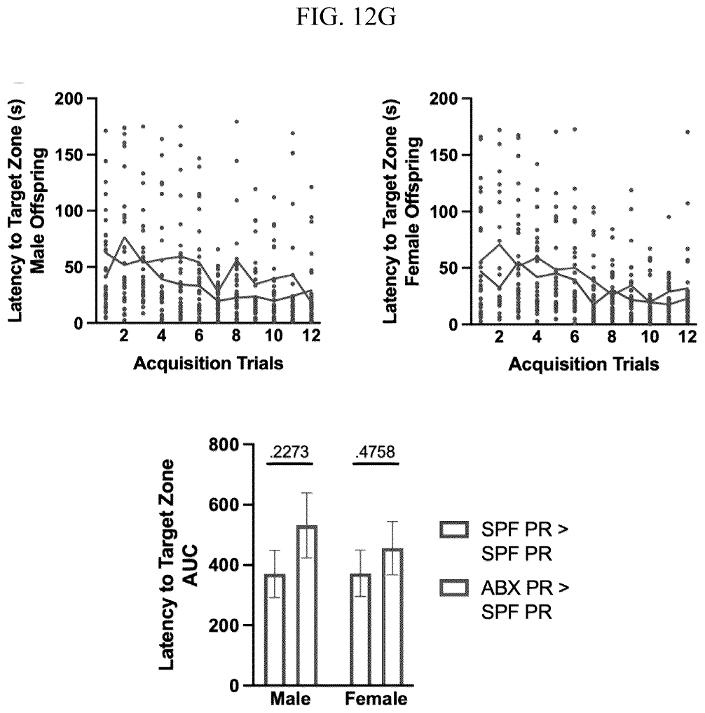

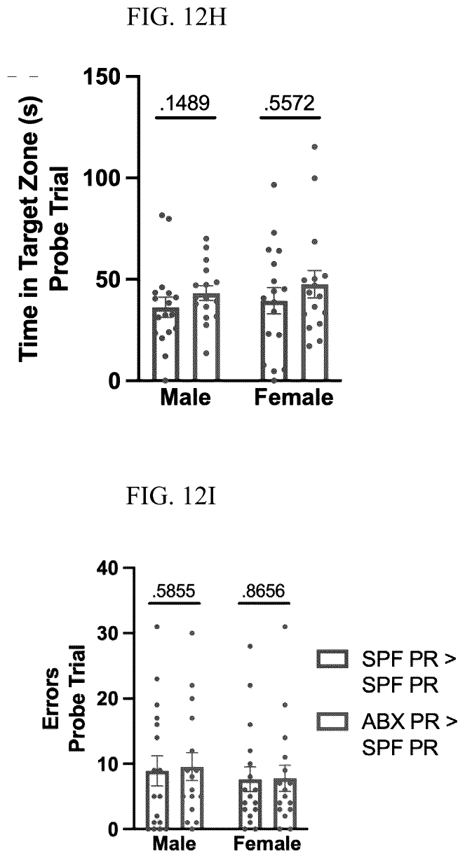

Maternal microbiome depletion and protein restriction during pregnancy yielded adult offspring with anxiety-like behavior that was not statistically different from that seen in control offspring from SPF and PR-fed dams (FIG. 12A, E). However, adult offspring of ABX PR dams exhibited exacerbated learning deficits, with increased latency to target zone during the acquisition phase of the Barnes maze assay relative to cognitively impaired control offspring of SPF dams fed PR (FIG. 10B). In other parameters of the Barnes maze test, offspring of microbiome-depleted and PR-fed dams exhibited deficiencies in performance that were not statistically significantly different from those seen in control offspring from PR-fed SPF dams (FIG. 12B-D). When analyzing data for male and female offspring separately, male offspring were especially affected by maternal microbiome depletion, with more pronounced exacerbation of cognitive behavior, which did not reach statistical significance (FIG. 12F-I). These results indicate that further reducing the diversity of the maternal microbiome, as shaped by protein restriction during pregnancy, yields offspring developmental programs that lead to deficient postnatal weight gain and exacerbated cognitive impairment during adulthood. These findings further suggest that interventions to improve the diversity and function of the maternal microbiome may aid in preventing the adverse effects of maternal protein undernutrition on offspring growth and behavior.

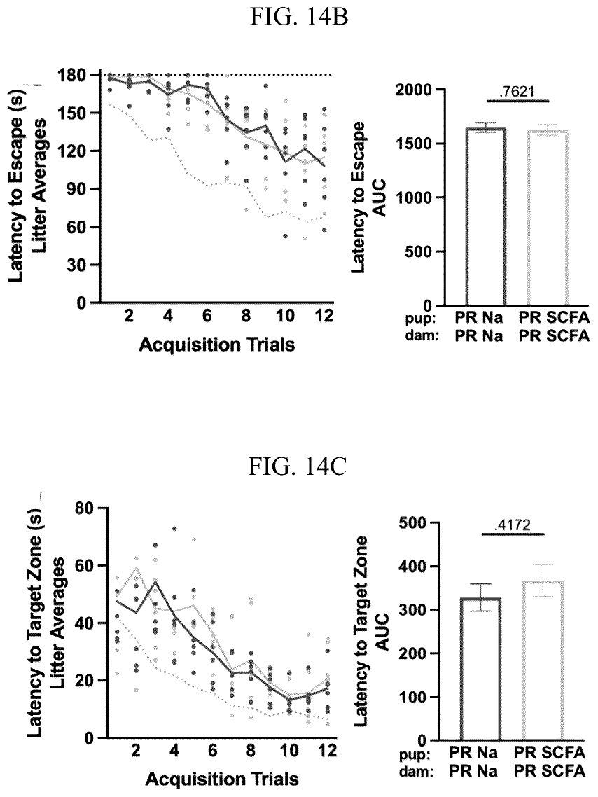

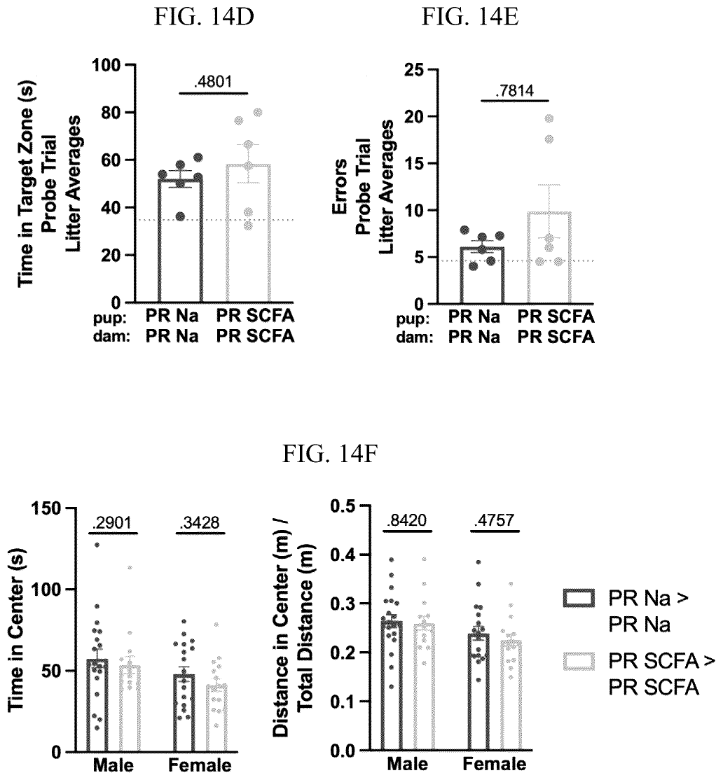

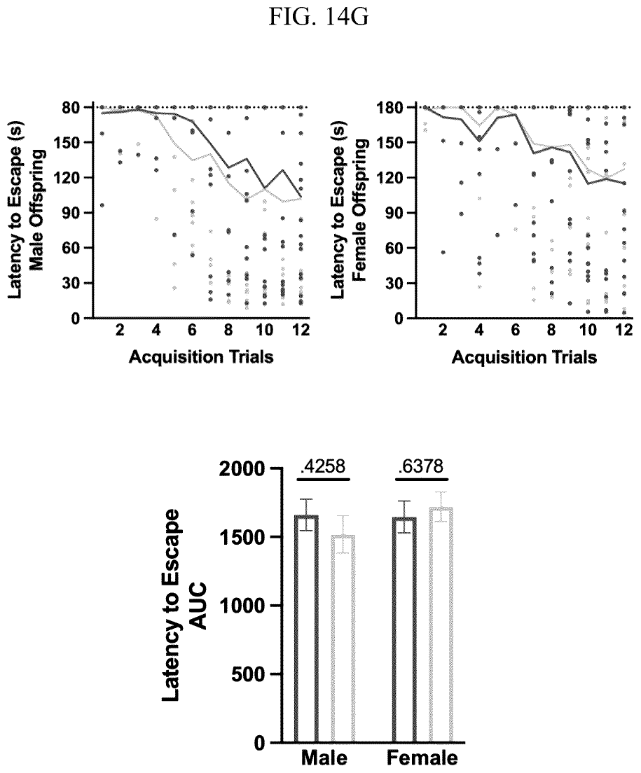

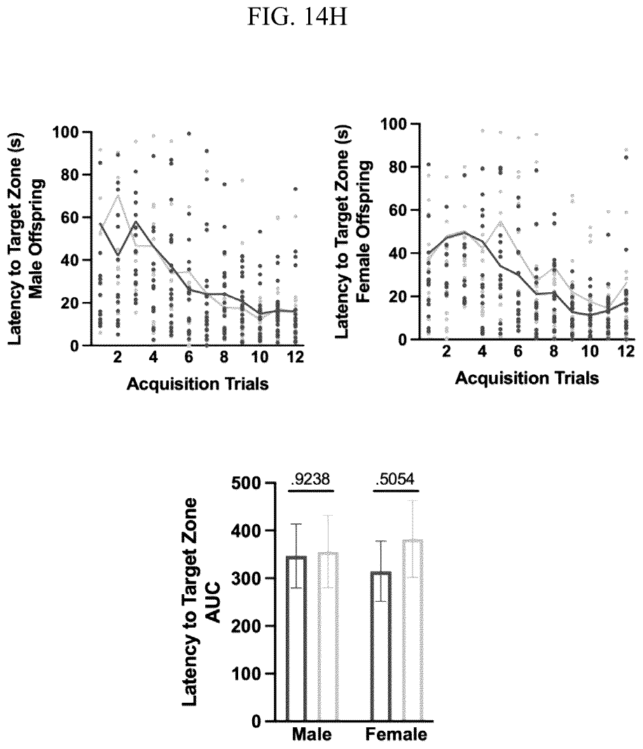

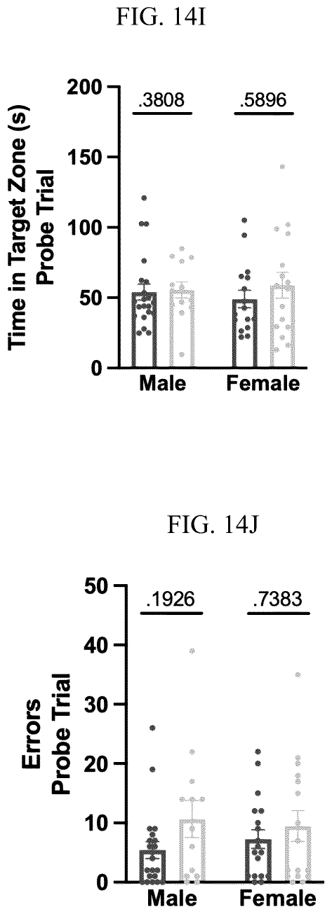

Short chain fatty acids (SCFAs) produced by bacterial fermentation of complex carbohydrates promote normal gastrointestinal and immune function, and their supplementation is reported to counter adverse effects of high fat or low fiber diets on behavioral development. To explore potential microbiome-based interventions for preventing adverse effects of maternal protein undernutrition on offspring growth and behavior, supplementation with SCFAs was first tested, which was previously utilized to prevent placental insufficiencies induced by maternal protein restriction. PR-fed dams were supplemented with a cocktail of SCFAs (acetate, butyrate, and propionate), or a sodium-matched vehicle control, in water throughout gestation (FIG. 13A). There were no significant effects of maternal SCFA supplementation during pregnancy on maternal PR-induced reductions in fetal and maternal weight and elevations in maternal serum corticosterone, despite significant effects of SCFA supplementation on increasing maternal dietary intake (FIG. 13B-I, FIG. 14A-E). Maternal SCFA supplementation had no statistically significant effects on litter size at birth, but modestly increased rates of litter survival postnatally, and promoted a gradual increase in pup weight over the first two weeks of life, which was reversed in adulthood (FIG. 13F-I). Consistent with previous observations (FIG. 2), adult offspring of vehicle-treated and gestational PR-fed dams exhibited anxiety-like behavior in the open field and cognitive impairment in the Barnes maze despite rearing since birth on CD (FIG. 14A-E). There were no statistically significant effects of maternal SCFA supplementation on the behavioral abnormalities induced by maternal PR during pregnancy (FIG. 14A-J). These results indicate that while maternal SCFA supplementation during pregnancy may effectively prevent adverse effects of maternal protein restriction on placental development and promote early postnatal growth (FIG. 13H), it fails to avert diet-induced neurodevelopmental programming of behavioral abnormalities in adult offspring. This aligns with human studies, wherein children subject to early life protein undernutrition remain at risk for mood and neurocognitive disorders despite subsequent nutritional rehabilitation and adequate postnatal growth.

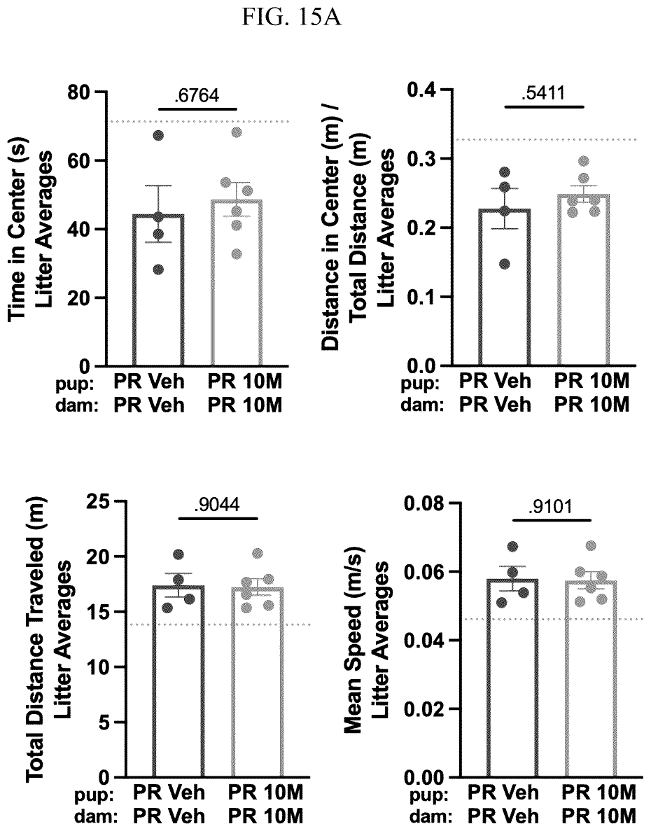

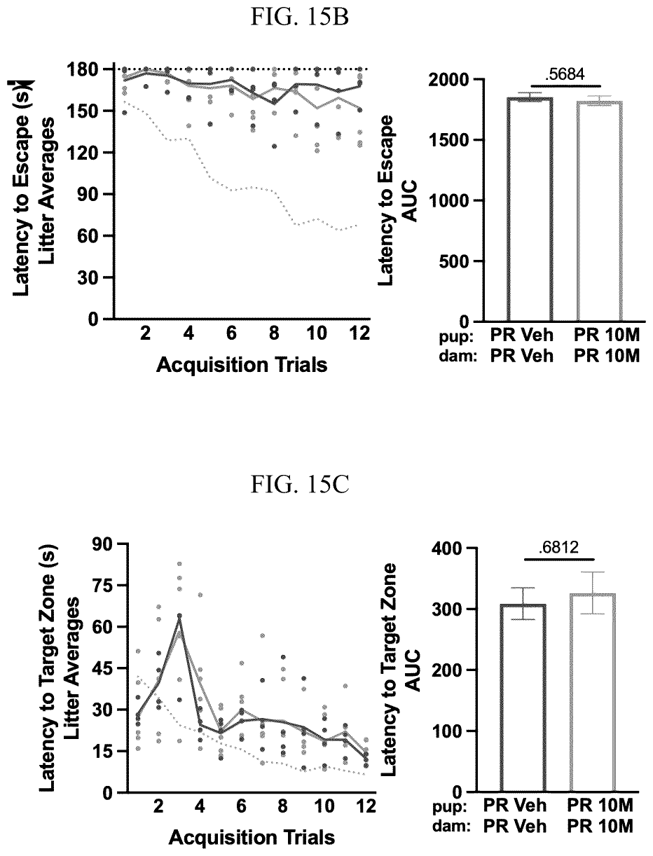

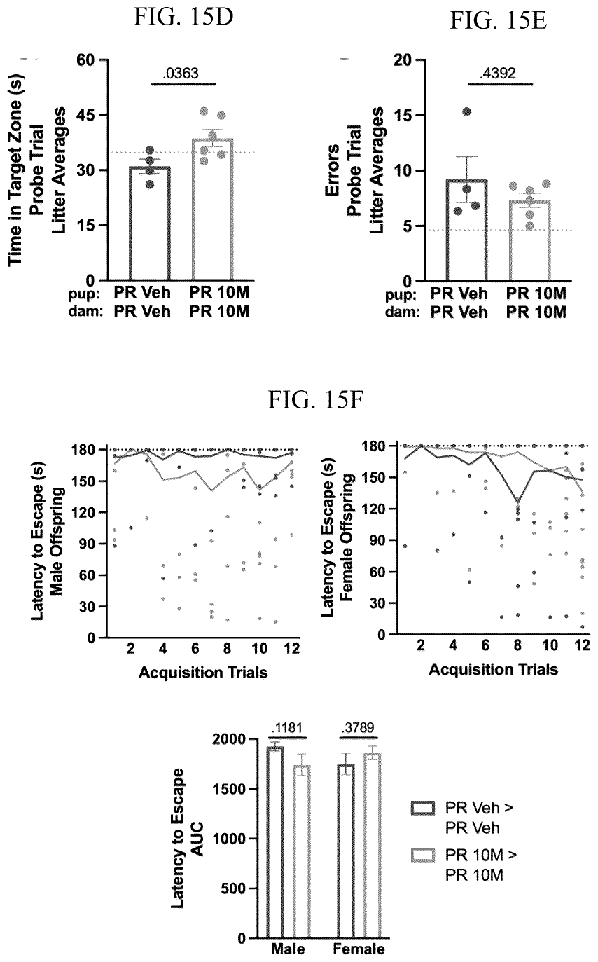

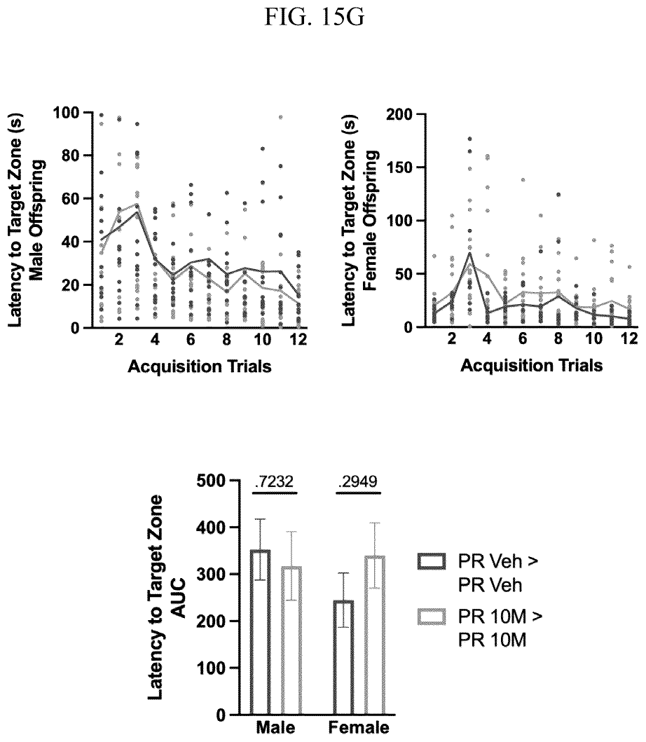

The maternal microbiome regulates numerous metabolites in the maternal blood, fetal blood, and fetal brain, a subset of which aid in guiding normal fetal neurodevelopment. Further, the possibility that microbial metabolites aside from SCFAs could be used to attenuate adverse effects of maternal protein undernutrition on offspring development was explored. To do so, 10 microbially-modulated metabolites (10M: 3-indoxul sulfate, phenylacetylglycine, imidazole propionate, alpha-hydroxyisocaproate, 2-hydroxy-3-methylvalerate, alpha-hydroxyisovalerate, 1-methylhistamine, beta-hydroxyisovalerate, 2R, 3R-dihydroxybutyrate, and N-acetylleucine) that were reduced in the fetal brain by maternal PR, similarly reduced by maternal PR in maternal serum, and additionally altered by maternal ABX treatment, were identified, indicating dependence on the maternal microbiome (FIG. 10C). Pregnant dams were fed PR and treated with 10M, or vehicle control, throughout gestation via a daily subcutaneous injection (FIG. 10D). There were no statistically significant effects of maternal 10M supplementation on PR-induced reductions in fetal weight, maternal weight, and maternal food consumption (FIG. 13J-L). However, maternal 10M supplementation significantly attenuated PR-induced increases in maternal serum corticosterone and drastically increased offspring survival (FIG. 13M,O), with no effect on litter size at birth, pup weight, or adult weight (FIG. 13N, P, Q). Adult offspring of vehicle-treated dams fed PR exhibited anxiety-like behavior in the open field and cognitive behavioral impairment in the Barnes maze (FIG. 15A-E), which reproduced effects of maternal protein undernutrition that were seen in initial PR experiments (FIG. 2) and in vehicle control groups for SCFA treatment (FIG. 14). Adult offspring of 10M-supplemented and PR-fed dams displayed sexually dimorphic behavioral responses relative to control offspring of vehicle-treated and PR-fed dams (FIG. 10E-H, FIG. 15F-G). In particular, female offspring of 10M-supplemented dams exhibited increased time and distance in the center during open field test without any motor effects (FIG. 10E-F, FIG. 15A), suggesting reduced anxiety-like behavior which aligns with the female-bias in anxiety-like behavior seen in response to maternal PR (FIG. 3B). In contrast, male offspring of 10M-supplemented dams exhibited increased time in target zone and decreased errors during the probe phase of Barnes maze (FIG. OG-H), suggesting better 24-hour recall without an effect on learning during the acquisition phase (FIG. 15B-E, FIG. 15F-G). These results indicate that maternal 1M supplementation during pregnancy partially attenuates adverse effects of maternal protein undernutrition on abnormal cognitive and anxiety-related behavior in adult offspring in a sex-specific manner. These sex- and domain-specific improvements are seen in the absence of normalized fetal or postnatal growth, again providing evidence that amelioration of neurobehavioral trajectories can occur independently of restoration of physical growth.

TABLE 3
Untargeted metabolomics of E18.5 fetal brains
Table of statistically significant biochemicals profiled in E18.5 fetal brains
collected from SPF CD, SPF PR, ABX CD, and ABX PR groups.

| | Fold Change | | | | | | |
|---|---|---|---|---|---|---|---|
| | | ABX PR | ABX CD | ABX PR | ANOVA Main Effects | | |
| | SPF PR vs. | vs. ABX | vs. SPF | vs. SPF | | | Treatment:Diet |
| Biochemical Name | SPF CD | CD | CD | PR | Treatment | Diet | interaction |
| glycine | 1.65 ++ | 1.35 ++ | 1.08 | 0.88 | | ** | |
| N-acetylglycine | 1.62 ++ | 1.48 ++ | 1.05 | 0.96 | | ** | |
| sarcosine | 0.55 −− | 0.35 −− | 1.16 | 0.72 | | ** | |
| dimethylglycine | 1.54 ++ | 1.24 ++ | 1.07 | 0.87 −− | | ** | ** |
| serine | 1.23 + | 1.09 | 1.10 | 0.97 | | ** | |
| 2-methylserine | 0.71 −− | 0.84 | 0.56 −− | 0.66 −− | ** | ** | |
| threonine | 0.29 −− | 0.4 −− | 1.35 ++ | 1.83 ++ | ** | ** | * |
| N-acetylthreonine | 0.83 −− | 0.84 −− | 1.13 ++ | 1.14 ++ | ** | ** | |
| allo-threonine | 0.13 −− | 0.17 −− | 1.46 | 1.88 | | ** | |
| N-methylalanine | 0.62 −− | 0.77 −− | 0.78 −− | 0.96 | * | ** | |
| aspartate | 0.85 −− | 0.81 −− | 1.00 | 0.95 | | ** | |
| asparagine | 1.28 + | 1.18 + | 0.97 | 0.90 | | ** | |
| N-acetylasparagine | 1.45 ++ | 1.32 ++ | 0.99 | 0.91 | | ** | |
| hydroxyasparagine** | 1.21 ++ | 1.16 ++ | 1.05 | 1.00 | | ** | |
| glutamine | 2.45 ++ | 2.1 ++ | 0.63 | 0.54 −− | ** | ** | |
| N-acetylglutamine | 1.39 ++ | 1.32 ++ | 1.00 | 0.94 | | ** | |
| 4-hydroxyglutamate | 0.96 | 1.18 | 0.83 − | 1.03 | | | |
| gamma-carboxyglutamate | 0.94 | 0.77 −− | 1.14 | 0.94 | | ** | * |
| glutamate, gamma-methyl ester | 1.14 ++ | 1.13 ++ | 0.99 | 0.97 | | ** | |
| beta-citrylglutamate | 1.04 + | 1.04 + | 1.02 | 1.02 | | ** | |
| gamma-aminobutyrate (GABA) | 1.02 | 1.1 ++ | 0.96 −− | 1.04 + | | ** | ** |
| carboxyethyl-GABA | 1.62 ++ | 1.39 ++ | 1.09 | 0.94 | | ** | |
| histidine | 0.6 −− | 0.68 −− | 0.87 | 0.98 | | ** | |
| 1-methylhistidine | 1.46 + | 1.79 ++ | 0.90 | 1.11 | | ** | |
| 3-methylhistidine | 4.29 ++ | 3.8 ++ | 1.10 | 0.98 | | ** | |
| N-acetylhistidine | 0.72 −− | 0.58 −− | 0.98 | 0.79 | | ** | |
| N-acetyl-1-methylhistidine* | 2.33 ++ | 1.81 ++ | 1.68 ++ | 1.30 | ** | ** | |
| trans-urocanate | 0.66 −− | 0.82 | 1.02 | 1.26 + | | ** | |
| imidazole propionate | 0.34 −− | 0.46 −− | 0.16 −− | 0.22 −− | ** | ** | |
| formiminoglutamate | 0.6 −− | 0.72 − | 0.92 | 1.12 | | ** | |
| imidazole lactate | 0.84 −− | 0.53 −− | 0.88 | 0.55 | | ** | |
| anserine | 1.18 + | 1.44 ++ | 0.95 | 1.16 | | ** | |
| histamine | 0.39 −− | 0.52 −− | 0.88 | 1.18 | | ** | |
| 1-methylhistamine | 0.56 −− | 0.78 −− | 0.88 | 1.21 + | | ** | ** |
| 1-methyl-4-imidazoleacetate | 0.74 −− | 0.90 | 0.95 | 1.16 | | ** | |
| 1-methyl-5-imidazoleacetate | 3.72 ++ | 2.78 ++ | 1.20 | 0.90 | | ** | |
| 1-methyl-5-imidazolelactate | 3 ++ | 2.43 ++ | 1.25 + | 1.01 | | ** | |
| 1-ribosyl-imidazoleacetate* | 0.68 −− | 0.76 −− | 0.90 | 1.01 | | ** | |
| 4-imidazoleacetate | 0.68 −− | 0.58 −− | 0.99 | 0.84 | | ** | |
| N2-acetyllysine | 1.16 + | 1.08 | 1.05 | 0.98 | | ** | |
| N6-acetyllysine | 1.08 | 1.29 ++ | 0.93 | 1.11 | | ** | |
| N6-methyllysine | 1.95 ++ | 1.86 ++ | 1.09 | 1.04 | | ** | |
| N6,N6-dimethyllysine | 1.13 + | 1.05 | 1.01 | 0.94 | | ** | |
| hydroxy-N6,N6,N6-trimethyllysine* | 0.9 − | 0.89 −− | 1.06 | 1.04 | | ** | |
| 5-hydroxylysine | 1.12 + | 1.04 | 1.13 ++ | 1.05 | * | | |
| saccharopine | 1.23 | 1.54 ++ | 0.84 | 1.05 | | ** | |
| 2-aminoadipate | 0.83 −− | 0.85 −− | 1.01 | 1.04 | | ** | |
| glutarylcarnitine (C5-DC) | 2.32 ++ | 2.4 ++ | 0.95 | 0.98 | | ** | |
| pipecolate | 1.10 | 1.42 ++ | 0.95 | 1.23 ++ | | ** | * |
| cadaverine | 0.95 | 1.12 | 1.03 | 1.22 + | | | |
| N-acetyl-cadaverine | 0.86 | 1.16 | 1.03 | 1.38 ++ | * | | |
| 5-aminovalerate | 1.72 + | 1.40 | 1.00 | 0.82 | | ** | |
| N,N,N-trimethyl-5-aminovalerate | 1.51 + | 0.97 | 0.93 | 0.6 −− | ** | | * |
| N-acetyl-2-aminoadipate | 0.99 | 0.77 −− | 1.14 + | 0.88 | | ** | ** |
| phenylalanine | 1.16 | 1.21 ++ | 0.87 | 0.92 | * | ** | |
| N-acetylphenylalanine | 1.4 + | 1.27 + | 0.88 | 0.80 | * | ** | |
| N-acetyltyrosine | 1.52 | 0.64 | 0.77 | 0.33 − | * | | |
| 3-methoxytyrosine | 2.01 ++ | 1.89 ++ | 0.76 | 0.72 | * | ** | |
| O-methyltyrosine | 1.11 + | 0.92 | 1.09 + | 0.91 − | | | ** |
| N-formylphenylalanine | 3.05 + | 1.39 | 1.32 | 0.60 | | * | |
| tryptophan | 1.35 ++ | 1.38 ++ | 0.99 | 1.01 | | ** | |
| N-acetyltryptophan | 1.78 ++ | 1.37 ++ | 1.14 | 0.88 | | ** | |
| C-glycosyltryptophan | 1.08 + | 1.07 | 0.99 | 0.99 | | ** | |
| oxindolylalanine | 1.55 + | 1.31 | 1.07 | 0.91 | | ** | |
| kynurenine | 1.46 ++ | 1.63 ++ | 0.86 | 0.96 | | ** | |
| N-acetylkynurenine (2) | 1.67 ++ | 1.71 ++ | 0.90 | 0.92 | | ** | |
| kynurenate | 1.32 | 1.64 ++ | 0.79 | 0.99 | | ** | |
| 5-hydroxyindoleacetate | 1.38 + | 1.36 + | 1.01 | 0.99 | | ** | |
| indoleacetate | 1.5 ++ | 1.46 ++ | 1.02 | 0.99 | | ** | |
| 3-indoxyl sulfate | 0.66 − | 0.56 | 0.33 −− | 0.28 −− | ** | * | |
| leucine | 0.77 −− | 0.94 | 1.02 | 1.25 ++ | | ** | |
| N-acetylleucine | 0.59 −− | 0.75 −− | 0.98 | 1.24 ++ | | ** | * |
| alpha-hydroxyisocaproate | 0.6 −− | 1.24 | 0.83 | 1.71 ++ | | | ** |
| isovalerate (i5:0) | 1.26 | 0.69 | 2.32 ++ | 1.28 | ** | | |
| isovalerylglycine | 0.56 −− | 0.39 −− | 1.01 | 0.72 | | ** | |
| isovalerylcarnitine (C5) | 0.63 −− | 0.75 | 1.02 | 1.22 | | ** | |
| beta-hydroxyisovalerate | 0.55 −− | 0.76 −− | 1.10 | 1.51 ++ | ** | ** | * |
| beta-hydroxyisovaleroylcarnitine | 0.5 −− | 0.53 −− | 1.12 + | 1.18 ++ | ** | ** | |
| isoleucine | 0.74 −− | 0.88 | 1.02 | 1.21 ++ | | ** | |
| N-acetylisoleucine | 0.77 −− | 0.89 | 1.05 | 1.21 ++ | ** | ** | |
| 2-hydroxy-3-methylvalerate | 0.68 − | 1.09 | 1.16 | 1.88 ++ | ** | | * |
| 2-methylbutyrylcarnitine (C5) | 0.61 −− | 0.80 | 1.08 | 1.39 ++ | * | ** | |
| tiglylcarnitine (C5:1-DC) | 0.41 −− | 0.48 −− | 1.05 | 1.23 | | ** | |
| ethylmalonate | 1.26 + | 0.99 | 1.2 ++ | 0.94 | | ** | ** |
| methylsuccinate | 1.2 + | 1.03 | 1.11 | 0.95 | | | |
| valine | 0.84 −− | 0.83 −− | 1.08 | 1.06 | | ** | |
| 3-methyl-2-oxobutyrate | 1.17 | 0.98 | 1.4 ++ | 1.17 | ** | | |
| alpha-hydroxyisovalerate | 0.68 −− | 1.10 | 0.99 | 1.61 ++ | ** | * | ** |
| isobutyrylcarnitine (C4) | 0.6 −− | 0.68 −− | 1.03 | 1.16 | | ** | |
| 3-hydroxyisobutyrate | 0.44 −− | 0.45 −− | 1.02 | 1.06 | | ** | |
| methionine | 0.78 −− | 0.71 −− | 0.89 | 0.82 | | ** | |
| methionine sulfone | 2.53 ++ | 4.92 ++ | 0.90 | 1.76 ++ | | ** | ** |
| S-adenosylhomocysteine (SAH) | 0.82 −− | 0.92 | 1.01 | 1.13 + | | ** | |
| 5-methylthioribose** | 1.35 + | 1.3 + | 0.96 | 0.93 | | ** | |
| 2,3-dihydroxy-5-methylthio-4- | 0.98 | 0.85 − | 0.99 | 0.85 | | | |
| pentenoate (DMTPA)* | | | | | | | |
| cystathionine | 0.64 −− | 0.48 −− | 1.15 | 0.87 | | ** | |
| cysteine | 0.56 −− | 0.91 | 1.02 | 1.66 ++ | * | ** | * |
| S-methylcysteine | 4.2 ++ | 4.02 ++ | 0.80 | 0.77 | ** | ** | |
| S-methylcysteine sulfoxide | 7.57 ++ | 6.31 ++ | 0.94 | 0.78 | | ** | |
| cystine | 0.54 −− | 0.71 | 1.77 | 2.34 ++ | ** | ** | |
| lanthionine | 0.36 −− | 0.49 −− | 1.32 | 1.8 ++ | ** | ** | |
| hypotaurine | 0.24 −− | 0.35 −− | 1.25 | 1.84 ++ | ** | ** | |
| taurocyamine | 2.08 ++ | 1.82 ++ | 1.35 ++ | 1.18 | ** | ** | |
| 3-sulfo-L-alanine | 0.72 − | 0.96 | 1.02 | 1.36 | | * | |
| urea | 0.1 −− | 0.22 −− | 1.04 | 2.27 ++ | ** | ** | ** |
| ornithine | 1.23 + | 0.93 | 1.12 | 0.85 | | | * |
| 3-amino-2-piperidone | 1.00 | 0.84 − | 1.12 | 0.94 | | | |
| 2-oxoarginine* | 0.68 −− | 0.42 −− | 1.14 | 0.71 | | ** | |
| citrulline | 1.28 + | 1.09 + | 1.07 | 0.91 − | | ** | ** |
| homoarginine | 1.68 ++ | 1.52 ++ | 1.02 | 0.92 | | ** | |
| homocitrulline | 1.91 ++ | 2.72 ++ | 1.00 | 1.43 ++ | ** | ** | ** |
| proline | 1.53 + | 1.3 + | 1.02 | 0.87 | | ** | |
| dimethylarginine (SDMA + ADMA) | 1.18 + | 1.29 ++ | 1.03 | 1.14 | | ** | |
| N-acetylarginine | 0.75 −− | 0.58 −− | 1.34 ++ | 1.04 | | ** | |
| N-acetylcitrulline | 1.37 + | 1.05 | 1.06 | 0.81 | | * | |
| N-delta-acetylornithine | 1.59 ++ | 1.48 ++ | 0.79 | 0.73 | ** | ** | |
| trans-4-hydroxyproline | 1.01 | 1.05 | 1.1 ++ | 1.15 ++ | ** | | |
| N,N,N-trimethyl-alanylproline | 1.05 | 1.11 ++ | 1.00 | 1.05 | | ** | |
| betaine (TMAP) | | | | | | | |
| argininate* | 0.41 −− | 0.48 −− | 1.00 | 1.15 | | ** | |
| guanidinoacetate | 0.82 | 0.67 −− | 1.13 | 0.93 | | ** | |
| creatine phosphate | 1.15 | 0.95 | 1.26 ++ | 1.05 | * | | |
| putrescine | 1.32 + | 1.10 | 1.19 ++ | 0.99 | * | ** | ** |
| N-acetylputrescine | 1.33 + | 1.18 + | 1.22 ++ | 1.08 | ** | ** | |
| spermidine | 1.04 | 1.14 + | 0.98 | 1.08 | | | |
| (N(1) + N(8))-acetylspermidine | 1.23 ++ | 1.32 ++ | 1.05 | 1.13 | | ** | |
| spermine | 0.92 | 1.19 | 0.92 | 1.19 + | | | |
| 4-guanidinobutanoate | 0.58 −− | 0.73 −− | 0.92 | 1.15 | | ** | |
| glutathione, oxidized (GSSG) | 0.73 −− | 0.81 − | 1.07 | 1.19 + | | ** | |
| cysteine-glutathione disulfide | 0.59 −− | 0.78 | 1.24 | 1.64 ++ | ** | ** | |
| S-methylglutathione | 1.37 + | 0.95 | 1.07 | 0.74 −− | | | * |
| cysteinylglycine | 0.68 −− | 0.94 | 1.12 | 1.54 ++ | ** | * | |
| 5-oxoproline | 1.42 ++ | 1.28 ++ | 0.99 | 0.89 | | ** | |
| 2-hydroxybutyrate/2-hydroxyisobutyrate | 0.85 | 1.3 + | 0.99 | 1.53 ++ | * | | * |
| ophthalmate | 2.32 + | 1.66 + | 0.86 | 0.62 | | ** | |
| S-(1,2-dicarboxyethyl)glutathione | 0.75 − | 0.53 −− | 1.09 | 0.76 | | ** | |
| CoA-glutathione* | 0.91 | 0.76 −− | 1.04 | 0.87 | | ** | |
| gamma-glutamylglutamate | 0.76 − | 0.80 | 0.99 | 1.05 | | ** | |
| gamma-glutamylglutamine | 1.38 + | 1.33 + | 1.09 | 1.06 | | ** | |
| gamma-glutamylglycine | 18.63 ++ | 5.29 ++ | 1.54 | 0.44 − | | ** | * |
| gamma-glutamylhistidine | 0.52 −− | 0.48 −− | 1.01 | 0.94 | | ** | |
| gamma-glutamylisoleucine* | 0.97 | 0.67 −− | 1.08 | 0.75 | | ** | |
| gamma-glutamyl-alpha-lysine | 0.91 | 0.73 −− | 1.18 + | 0.95 | | ** | * |
| gamma-glutamyl-epsilon-lysine | 0.93 | 0.82 −− | 1.05 | 0.93 | | ** | |
| gamma-glutamylmethionine | 0.71 −− | 0.61 −− | 0.97 | 0.83 | | ** | |
| gamma-glutamylthreonine | 0.48 −− | 0.47 −− | 1.26 | 1.22 | * | ** | |
| gamma-glutamyltryptophan | 1.24 | 1.44 ++ | 1.02 | 1.18 | | ** | |
| gamma-glutamyltyrosine | 1.23 | 0.46 − | 0.97 | 0.36 | | ** | |
| gamma-glutamylvaline | 1.99 + | 1.06 | 1.03 | 0.55 − | | | |
| gamma-glutamylserine | 1.71 + | 1.19 | 1.21 | 0.84 | | ** | |
| alanylleucine | 0.66 − | 1.53 + | 0.73 | 1.71 ++ | | | ** |
| glycylleucine | 0.92 | 1.36 + | 0.93 | 1.36 | | | |
| isoleucylglycine | 1.11 | 1.6 + | 1.16 | 1.66 + | | | |
| leucylglycine | 0.86 | 1.36 | 0.95 | 1.51 ++ | | | * |
| valylleucine | 0.78 | 1.38 + | 0.98 | 1.73 ++ | ** | | ** |
| leucylglutamine* | 1.02 | 1.52 ++ | 1.22 | 1.83 ++ | ** | | |
| phenylacetylglutamate | 0.95 | 1.39 | 0.37 −− | 0.55 −− | ** | | |
| phenylacetylglycine | 0.74 − | 1.49 ++ | 0.3 −− | 0.6 −− | ** | | ** |
| N,N-dimethyl-pro-pro | 1.07 + | 1.02 | 1.05 | 1.00 | | | |
| 1,5-anhydroglucitol (1,5-AG) | 1.19 ++ | 1.48 ++ | 1.10 | 1.36 ++ | ** | ** | ** |
| fructose 1,6-diphosphate/glucose | 1.05 | 0.73 | 0.93 | 0.65 − | | | |
| 1,6-diphosphate/myo-inositol | | | | | | | |
| diphosphates | | | | | | | |
| pyruvate | 0.88 − | 0.81 −− | 1.03 | 0.95 | | ** | |
| lactate | 0.98 | 1.14 + | 0.93 | 1.08 | | | |
| sedoheptulose-7-phosphate | 1.33 + | 0.84 | 1.28 | 0.81 | | | * |
| arabonate/xylonate | 1.12 + | 1.10 | 1.04 | 1.02 | | ** | |
| maltotetraose | 0.92 | 1.34 + | 0.87 | 1.26 | | | * |
| maltose | 0.94 | 1.6 + | 0.83 | 1.41 | | | |
| sucrose | 2.29 ++ | 2.77 ++ | 0.84 | 1.01 | | ** | |
| mannose | 0.89 | 0.83 | 1.27 + | 1.18 | ** | | |
| UDP-glucose | 0.96 | 0.83 −− | 1.06 | 0.92 | | ** | * |
| UDP-galactose | 0.94 | 0.86 −− | 1.04 | 0.96 | | ** | |
| UDP-glucuronate | 0.92 | 0.83 −− | 1.06 | 0.96 | | ** | |
| UDP-N- | 1.11 + | 0.97 | 1.02 | 0.89 −− | | | |
| acetylglucosamine/galactosamine | | | | | | | |
| N-acetylneuraminate | 0.89 − | 1.02 | 0.87 −− | 1.00 | | | |
| erythronate* | 1.14 + | 1.02 | 1.07 | 0.96 | | | |
| N6-carboxymethyllysine | 0.95 | 0.82 | 1.3 + | 1.12 | ** | | |
| succinylcarnitine (C4-DC) | 0.6 −− | 1.10 | 0.54 −− | 0.99 | ** | | ** |
| malonylcarnitine | 1.5 + | 1.21 + | 1.12 | 0.90 | | ** | |
| acetyl COA | 2.01 | 1.46 | 0.86 | 0.63 − | * | | |
| caproate (6:0) | 0.78 − | 1.04 | 0.85 | 1.13 | | | |
| caprylate (8:0) | 1.13 | 1.02 | 1.3 + | 1.18 | ** | | |
| pelargonate (9:0) | 0.97 | 1.18 | 0.78 −− | 0.95 | * | | |
| caprate (10:0) | 1.17 | 0.76 | 1.46 ++ | 0.95 | | | |
| tetradecadienoate (14:2)* | 1.47 ++ | 1.24 ++ | 1.18 | 0.99 | | ** | |
| eicosapentaenoate (EPA; 20:5n3) | 1.21 | 1.38 + | 1.04 | 1.19 | | * | |
| docosapentaenoate (n3 DPA; 22:5n3) | 1.91 ++ | 2.01 ++ | 1.11 | 1.16 | | ** | |
| docosahexaenoate (DHA; 22:6n3) | 1.30 | 1.42 + | 1.17 | 1.29 | | ** | |
| nisinate (24:6n3) | 1.65 ++ | 1.77 ++ | 1.23 | 1.31 | | ** | |
| linoleate (18:2n6) | 1.29 | 1.49 ++ | 0.99 | 1.14 | | ** | |
| linolenate [alpha or gamma; | 1.46 ++ | 1.52 ++ | 0.97 | 1.01 | | ** | |
| (18:3n3 or 6)] | | | | | | | |
| dihomo-linolenate (20:3n3 or n6) | 1.67 ++ | 1.66 ++ | 1.13 | 1.12 | | ** | |
| docosatrienoate (22:3n6)* | 0.75 − | 0.86 | 0.92 | 1.05 | | * | |
| glutarate (C5-DC) | 1.18 | 1.28 ++ | 1.05 | 1.14 | | ** | |
| 2-aminoheptanoate | 6.59 ++ | 5.49 ++ | 1.07 | 0.89 | | ** | |
| 2-aminooctanoate | 3.49 ++ | 2.32 ++ | 1.34 | 0.89 | | ** | |
| propionylcarnitine (C3) | 0.84 − | 1.04 | 0.98 | 1.22 + | | | |
| hexanoylglycine | 4.09 ++ | 5.53 ++ | 0.94 | 1.27 | | ** | |
| acetylcarnitine (C2) | 1.38 + | 1.29 + | 1.00 | 0.94 | | ** | |
| nonanoylcarnitine (C9) | 0.96 | 1.65 ++ | 0.41 −− | 0.70 | ** | | * |
| laurylcarnitine (C12) | 1.96 ++ | 1.51 ++ | 1.24 | 0.96 | | ** | |
| myristoylcarnitine (C14) | 1.6 ++ | 1.35 ++ | 1.18 | 0.99 | | ** | |
| pentadecanoylcarnitine (C15)* | 1.46 + | 1.3 + | 1.27 | 1.13 | * | ** | |
| palmitoylcarnitine (C16) | 1.38 ++ | 1.31 ++ | 1.11 | 1.06 | | ** | |
| stearoylcarnitine (C18) | 1.39 ++ | 1.36 ++ | 1.14 | 1.12 | * | ** | |
| arachidoylcarnitine (C20)* | 1.3 + | 1.6 ++ | 1.07 | 1.32 ++ | * | ** | |
| cis-4-decenoylcarnitine (C10:1) | 1.7 + | 1.42 | 1.05 | 0.87 | | ** | |
| 5-dodecenoylcarnitine (C12:1) | 1.88 + | 1.44 + | 1.20 | 0.92 | | ** | |
| myristoleoylcarnitine (C14:1)* | 1.99 + | 1.42 + | 1.38 + | 0.98 | | ** | * |
| palmitoleoylcarnitine (C16:1)* | 1.64 ++ | 1.44 ++ | 1.18 | 1.03 | | ** | |
| oleoylcarnitine (C18:1) | 1.42 ++ | 1.42 ++ | 1.03 | 1.03 | | ** | |
| eicosenoylcarnitine (C20:1)* | 1.19 + | 1.35 ++ | 1.03 | 1.16 | | ** | |
| linoleoylcarnitine (C18:2)* | 2.07 ++ | 1.91 ++ | 1.08 | 1.00 | | ** | |
| linolenoylcarnitine (C18:3)* | 2.59 ++ | 2.03 ++ | 1.22 | 0.96 | | ** | |
| dihomo-linoleoylcarnitine (C20:2)* | 1.54 ++ | 1.62 ++ | 1.03 | 1.08 | | ** | |
| arachidonoylcarnitine (C20:4) | 1.69 ++ | 1.46 ++ | 1.29 + | 1.11 | * | ** | |
| docosatrienoylcarnitine (C22:3)* | 1.9 ++ | 1.86 ++ | 1.18 | 1.16 | | ** | |
| adrenoylcarnitine (C22:4)* | 1.93 ++ | 1.67 ++ | 1.35 ++ | 1.17 | ** | ** | |
| docosahexaenoylcarnitine (C22:6)* | 2.2 ++ | 2.12 ++ | 1.38 | 1.33 | ** | ** | |
| (R)-3-hydroxybutyrylcarnitine | 1.34 + | 1.63 ++ | 1.16 | 1.4 + | * | ** | |
| (S)-3-hydroxybutyrylcarnitine | 1.34 ++ | 1.37 ++ | 1.13 | 1.16 + | ** | ** | |
| 3-hydroxyhexanoylcarnitine (1) | 1.38 ++ | 1.49 ++ | 1.02 | 1.10 | | ** | |
| 3-hydroxyoctanoylcarnitine (1) | 1.35 ++ | 1.42 ++ | 0.96 | 1.01 | | ** | |
| 3-hydroxyoctanoylcarnitine (2) | 1.3 ++ | 1.24 ++ | 1.11 | 1.06 | | ** | |
| 3-hydroxypalmitoylcarnitine | 1.63 + | 1.27 + | 1.28 | 1.00 | | ** | |
| 3-hydroxyoleoylcarnitine | 1.44 + | 1.25 | 1.18 | 1.03 | | ** | |
| carnitine | 1.42 ++ | 1.28 ++ | 1.12 | 1.01 | | ** | |
| 3-hydroxybutyrate (BHBA) | 0.67 | 4.1 ++ | 0.68 | 4.16 ++ | | | ** |
| oleoylcholine | 1.33 | 0.81 | 1.71 ++ | 1.04 | * | | * |
| 2-hydroxydecanoate | 0.52 −− | 0.96 | 0.76 | 1.42 | | | |
| 3-hydroxyhexanoate | 1.23 | 1.6 ++ | 1.05 | 1.37 | | ** | |
| 13-HODE + 9-HODE | 1.07 | 2.19 ++ | 0.72 | 1.47 | | | |
| 2S,3R-dihydroxybutyrate | 0.64 −− | 1.04 | 1.01 | 1.63 ++ | ** | ** | ** |
| 2R,3R-dihydroxybutyrate | 0.35 −− | 0.48 −− | 1.33 ++ | 1.86 ++ | ** | ** | ** |
| 2,4-dihydroxybutyrate | 1.33 ++ | 1.45 ++ | 1.17 + | 1.28 ++ | ** | ** | |
| 3,4-dihydroxybutyrate | 1.25 + | 1.14 | 1.03 | 0.93 | | * | |
| palmitoyl ethanolamide | 1.06 | 1.21 ++ | 1.00 | 1.13 + | | ** | |
| stearoyl ethanolamide | 1.13 | 1.2 ++ | 1.02 | 1.08 | | ** | |
| docosahexaenoyl ethanolamide | 0.5 −− | 0.70 | 0.89 | 1.23 | | ** | |
| N-arachidonoyltaurine | 1.76 + | 0.95 | 2.05 ++ | 1.12 | ** | | |
| myo-inositol | 1.05 | 1.11 ++ | 0.94 | 1.00 | | ** | |
| choline phosphate | 1.22 + | 1.03 | 1.08 | 0.92 | | ** | * |
| phosphoethanolamine | 1.07 + | 1.03 | 1.00 | 0.96 | | * | |
| cytidine-5′-diphosphoethanolamine | 0.97 | 0.92 −− | 1.01 | 0.96 | | * | |
| glycerophosphoethanolamine | 0.72 −− | 0.71 −− | 0.89 | 0.88 | * | ** | |
| glycerophosphoserine* | 0.74 −− | 0.96 | 0.79 −− | 1.02 | * | ** | * |
| trimethylamine N-oxide | 1.24 | 0.12 | 0.13 −− | 0.01 −− | ** | | |
| 1-myristoyl-2-arachidonoyl-GPC | 1.29 ++ | 1.09 ++ | 1.18 ++ | 1.00 | ** | ** | ** |
| (14:0/20:4)* | | | | | | | |
| 1-palmitoyl-2-stearoyl-GPC | 1.08 | 1.1 + | 1.00 | 1.02 | | ** | |
| (16:0/18:0) | | | | | | | |
| 1-palmitoyl-2-gamma- | 1.12 + | 1.07 | 0.99 | 0.94 | | ** | |
| linolenoyl-GPC (16:0/18:3n6)* | | | | | | | |
| 1-palmitoyl-2-dihomo- | 1.24 ++ | 1.23 ++ | 1.02 | 1.02 | | ** | |
| linolenoyl-GPC (16:0/20:3n3 or 6)* | | | | | | | |
| 1-palmitoyl-2-arachidonoyl-GPC | 1.14 ++ | 1.07 ++ | 1.09 ++ | 1.02 | ** | ** | ** |
| (16:0/20:4n6) | | | | | | | |
| 1-palmitoyl-2-docosahexaenoyl- | 1.18 + | 1.13 + | 1.16 ++ | 1.11 | ** | ** | |
| GPC (16:0/22:6) | | | | | | | |
| 1-stearoyl-2-arachidonoyl-GPC | 1.17 ++ | 1.11 ++ | 1.13 ++ | 1.06 ++ | ** | ** | |
| (18:0/20:4) | | | | | | | |
| 1-stearoyl-2-docosahexaenoyl- | 1.18 + | 1.09 | 1.16 + | 1.07 | * | * | |
| GPC (18:0/22:6) | | | | | | | |
| 1,2-dioleoyl-GPC (18:1/18:1) | 0.93 − | 0.95 | 1.01 | 1.03 | | ** | |
| 1-oleoyl-2-linoleoyl-GPC | 1.39 ++ | 1.37 ++ | 0.98 | 0.96 | | ** | |
| (18:1/18:2)* | | | | | | | |
| 1-oleoyl-2-docosahexaenoyl- | 1.13 ++ | 1.11 ++ | 1.1 + | 1.09 | ** | ** | |
| GPC (18:1/22:6)* | | | | | | | |
| 1,2-dilinoleoyl-GPC (18:2/18:2) | 2.3 ++ | 1.98 ++ | 1.2 + | 1.03 | | ** | |
| 1-linoleoyl-2-arachidonoyl-GPC | 1.82 ++ | 1.57 ++ | 1.17 ++ | 1.01 | ** | ** | * |
| (18:2/20:4n6)* | | | | | | | |
| 1-palmitoyl-2-oleoyl-GPE | 0.99 | 1.01 | 1.03 | 1.05 + | ** | | |
| (16:0/18:1) | | | | | | | |
| 1-palmitoyl-2-linoleoyl-GPE | 1.04 | 1.13 + | 0.99 | 1.07 | | * | |
| (16:0/18:2) | | | | | | | |
| 1-palmitoyl-2-arachidonoyl-GPE | 1.08 ++ | 1.06 ++ | 1.06 ++ | 1.04 + | ** | ** | |
| (16:0/20:4)* | | | | | | | |
| 1-stearoyl-2-arachidonoyl-GPE | 1.06 ++ | 1.1 ++ | 1.03 | 1.06 ++ | ** | ** | |
| (18:0/20:4) | | | | | | | |
| 1-oleoyl-2-linoleoyl-GPE | 1.31 ++ | 1.69 ++ | 0.97 | 1.25 + | | ** | |
| (18:1/18:2)* | | | | | | | |
| 1-oleoyl-2-docosahexaenoyl- | 1.08 | 1.15 + | 1.05 | 1.12 | | * | |
| GPE (18:1/22:6)* | | | | | | | |
| 1-linoleoyl-2-arachidonoyl-GPE | 1.89 ++ | 1.57 ++ | 1.07 | 0.89 | | ** | |
| (18:2/20:4)* | | | | | | | |
| 1-palmitoyl-2-oleoyl-GPS | 1.09 + | 1.06 | 1.07 | 1.04 | * | ** | |
| (16:0/18:1) | | | | | | | |
| 1-stearoyl-2-oleoyl-GPS | 1.07 | 1.06 | 1.12 ++ | 1.11 + | ** | * | |
| (18:0/18:1) | | | | | | | |
| 1-stearoyl-2-arachidonoyl-GPS | 1.15 ++ | 1.15 ++ | 1.08 + | 1.08 + | ** | ** | |
| (18:0/20:4) | | | | | | | |
| 1-palmitoyl-2-oleoyl-GPG | 0.96 | 0.94 | 1.09 ++ | 1.08 + | ** | * | |
| (16:0/18:1) | | | | | | | |
| 1-stearoyl-2-arachidonoyl-GPI | 1.08 + | 1.02 | 1.18 ++ | 1.11 ++ | ** | * | |
| (18:0/20:4) | | | | | | | |
| 1-oleoyl-2-arachidonoyl-GPI | 1.02 | 0.96 | 1.12 ++ | 1.06 | ** | | |
| (18:1/20:4)* | | | | | | | |
| 1-palmitoleoyl-GPC (16:1)* | 1.05 | 1.04 | 1.11 + | 1.1 + | ** | | |
| 1-stearoyl-GPC (18:0) | 1.13 + | 1.09 | 1.09 | 1.05 | | ** | |
| 1-linoleoyl-GPC (18:2) | 1.24 | 1.46 ++ | 0.89 | 1.05 | | ** | |
| 1-stearoyl-GPE (18:0) | 0.97 | 1.08 ++ | 0.97 | 1.08 ++ | | | ** |
| 1-oleoyl-GPE (18:1) | 0.9 −− | 0.99 | 0.98 | 1.08 | | | |
| 1-(1-enyl-palmitoyl)-2- | 1.12 + | 1.17 ++ | 1.02 | 1.06 | | ** | |
| palmitoyl-GPC (P-16:0/16:0)* | | | | | | | |
| 1-(1-enyl-palmitoyl)-2- | 1.07 | 1.15 + | 0.95 | 1.01 | | * | |
| palmitoleoyl-GPC (P-16:0/16:1)* | | | | | | | |
| 1-(1-enyl-palmitoyl)-2- | 1.08 ++ | 1.06 ++ | 1.06 ++ | 1.04 | ** | ** | |
| arachidonoyl-GPE (P-16:0/20:4)* | | | | | | | |
| 1-(1-enyl-palmitoyl)-2-oleoyl-GPC | 1.05 | 1.23 ++ | 1.00 | 1.16 ++ | | ** | |
| (P-16:0/18:1)* | | | | | | | |
| 1-(1-enyl-stearoyl)-2- | 1.14 ++ | 1.13 ++ | 1.09 ++ | 1.08 + | ** | ** | |
| arachidonoyl-GPE (P-18:0/20:4)* | | | | | | | |
| 1-(1-enyl-palmitoyl)-GPE (P-16:0)* | 0.94 − | 1.03 | 0.98 | 1.07 ++ | | | * |
| glycerol | 1.16 | 1.35 + | 0.94 | 1.10 | | * | |
| 1-linoleoylglycerol (18:2) | 1.64 ++ | 1.61 ++ | 0.97 | 0.95 | | ** | |
| 2-myristoylglycerol (14:0) | 0.89 | 1.07 | 0.75 −− | 0.91 | ** | | |
| 2-palmitoylglycerol (16:0) | 0.85 − | 0.97 | 0.78 −− | 0.89 | ** | | |
| 2-linoleoylglycerol (18:2) | 1.53 ++ | 1.56 ++ | 0.82 | 0.84 | | ** | |
| palmitoyl-oleoyl-glycerol | 1.38 + | 1.18 | 1.31 | 1.12 | | ** | |
| (16:0/18:1) [2]* | | | | | | | |
| palmitoyl-linoleoyl-glycerol | 2.44 ++ | 1.72 ++ | 1.34 + | 0.95 | | ** | |
| (16:0/18:2) [2]* | | | | | | | |
| palmitoyl-arachidonoyl-glycerol | 1.33 + | 1.18 | 1.12 | 1.00 | | ** | |
| (16:0/20:4) [1]* | | | | | | | |
| palmitoyl-docosahexaenoyl- | 1.62 ++ | 1.5 ++ | 1.34 ++ | 1.23 ++ | ** | ** | |
| glycerol (16:0/22:6) [1]* | | | | | | | |
| palmitoyl-docosahexaenoyl- | 1.5 ++ | 1.54 ++ | 1.23 | 1.27 + | ** | ** | |
| glycerol (16:0/22:6) [2]* | | | | | | | |
| oleoyl-linoleoyl-glycerol | 1.64 ++ | 2.63 ++ | 0.7 − | 1.12 | | ** | * |
| (18:1/18:2) [2] | | | | | | | |
| stearoyl-docosahexaenoyl- | 0.95 | 1.01 | 1.15 | 1.22 + | ** | | |
| glycerol (18:0/22:6) [1]* | | | | | | | |
| stearoyl-docosahexaenoyl- | 1.23 | 1.29 ++ | 1.07 | 1.11 | | ** | |
| glycerol (18:0/22:6) [2]* | | | | | | | |
| phytosphingosine | 1.17 + | 1.10 | 0.99 | 0.93 | | ** | |
| N-palmitoyl-sphinganine | 0.79 −− | 0.96 | 1.03 | 1.26 ++ | ** | ** | * |
| (d18:0/16:0) | | | | | | | |
| N-stearoyl-sphinganine | 0.79 −− | 0.93 | 1.00 | 1.17 + | | ** | |
| (d18:0/18:0)* | | | | | | | |
| N-stearoyl-sphingadienine | 1.03 | 1.16 ++ | 1.01 | 1.14 ++ | * | * | |
| (d18:2/18:0)* | | | | | | | |
| ceramide (d18:1/17:0, | 0.66 −− | 0.84 | 0.85 | 1.08 | | ** | |
| d17:1/18:0)* | | | | | | | |
| ceramide (d18:2/24:1, | 1.23 ++ | 1.4 ++ | 0.99 | 1.13 | | ** | |
| d18:1/24:2)* | | | | | | | |
| glycosyl-N-palmitoyl- | 1.10 | 0.93 | 1.17 ++ | 0.99 | | | * |
| sphingosine (d18:1/16:0) | | | | | | | |
| glycosyl-N-stearoyl-sphingosine | 1.15 + | 1.07 | 1.04 | 0.96 | | ** | |
| (d18:1/18:0) | | | | | | | |
| palmitoyl dihydrosphingomyelin | 0.96 | 1.00 | 1.11 + | 1.15 ++ | ** | | |
| (d18:0/16:0)* | | | | | | | |
| sphingomyelin (d18:0/18:0, | 0.97 | 0.98 | 1.12 + | 1.13 + | ** | | |
| d19:0/17:0)* | | | | | | | |
| sphingomyelin (d18:0/20:0, | 0.86 | 1.06 | 1.17 | 1.44 ++ | ** | | |
| d16:0/22:0)* | | | | | | | |
| palmitoyl sphingomyelin | 1.14 + | 1.07 | 1.06 | 0.99 | | ** | |
| (d18:1/16:0) | | | | | | | |
| stearoyl sphingomyelin | 1.1 ++ | 1.12 ++ | 0.99 | 1.00 | | ** | |
| (d18:1/18:0) | | | | | | | |
| sphingomyelin (d18:1/14:0, | 1.13 + | 1.1 + | 0.99 | 0.97 | | ** | |
| d16:1/16:0)* | | | | | | | |
| sphingomyelin (d18:2/16:0, | 1.17 + | 1.06 | 1.10 | 1.00 | | ** | |
| d18:1/16:1)* | | | | | | | |
| sphingomyelin (d18:1/20:0, | 1.13 + | 1.11 | 1.05 | 1.04 | | ** | |
| d16:1/22:0)* | | | | | | | |
| sphingomyelin (d18:1/24:1, | 1.13 + | 1.13 + | 1.03 | 1.03 | | ** | |
| d18:2/24:0)* | | | | | | | |
| sphingomyelin (d18:2/24:1, | 1.67 ++ | 1.48 ++ | 1.12 | 1.00 | | ** | |
| d18:1/24:2)* | | | | | | | |
| sphingosine | 1.12 + | 1.09 + | 1.02 | 0.99 | | ** | |
| eicosanoylsphingosine (d20:1)* | 1.46 + | 1.07 | 1.25 | 0.91 | | * | |
| desmosterol | 1.1 + | 0.98 | 1.12 ++ | 1.00 | * | | * |
| cholesterol | 1.08 + | 1.1 + | 1.1 ++ | 1.12 ++ | ** | ** | |
| cholesterol sulfate | 1.09 | 0.74 −− | 1.25 + | 0.85 | | | ** |
| 4-cholesten-3-one | 1.02 | 0.85 −− | 1.06 | 0.88 −− | | * | ** |
| corticosterone | 2.76 ++ | 2.8 ++ | 1.13 | 1.15 | | ** | |
| taurocholate | 1.37 | 0.88 | 1.10 | 0.7 − | | | |
| tauro-beta-muricholate | 3.05 + | 0.83 | 1.15 | 0.31 −− | ** | * | ** |
| AICA ribonucleotide | 1.09 | 0.89 | 1.62 ++ | 1.33 | ** | | |
| urate | 1.63 ++ | 2.35 ++ | 0.96 | 1.39 | | ** | |
| allantoin | 1.21 ++ | 1.19 ++ | 1.06 | 1.04 | | ** | |
| allantoic acid | 1.17 ++ | 1.2 ++ | 1.05 | 1.08 | | ** | |
| adenosine 3′,5′-cyclic | 1.2 + | 0.94 | 1.05 | 0.82 − | | | * |
| monophosphate (cAMP) | | | | | | | |
| adenylosuccinate | 1.17 | 0.71 − | 1.39 | 0.84 | | | |
| adenine | 1.13 | 0.89 | 1.21 ++ | 0.95 | | | ** |
| N6-carbamoylthreonyladenosine | 1.07 + | 0.94 | 1.12 ++ | 0.98 | | | ** |
| 2′-deoxyadenosine 5′- | 0.97 | 0.63 −− | 1.09 | 0.70 | | * | |
| monophosphate | | | | | | | |
| methylthioadenosine sulfoxide | 1.26 + | 1.21 | 0.99 | 0.95 | | ** | |
| guanosine 5′-diphosphate (GDP) | 1.02 | 0.67 −− | 1.10 | 0.72 − | | | * |
| guanosine 5′-monophosphate (5′-GMP) | 0.92 − | 1.02 | 0.94 | 1.04 | | | |
| guanine | 1.06 | 1.18 | 1.10 | 1.22 + | ** | | |
| 7-methylguanine | 0.94 | 0.99 | 0.93 − | 0.97 | * | | |
| N2,N2-dimethylguanosine | 1.02 | 0.85 −− | 1.23 ++ | 1.02 | * | | |
| 2′-deoxyguanosine | 1.18 ++ | 1.19 ++ | 1.07 | 1.07 | | ** | |
| queuine | 0.99 | 0.41 −− | 0.82 | 0.33 −− | ** | ** | ** |
| orotidine | 0.91 −− | 0.85 −− | 1.05 | 0.98 | | ** | |
| uridine 5′-triphosphate (UTP) | 1.13 | 0.58 −− | 1.33 | 0.68 | | | * |
| uridine 5′-diphosphate (UDP) | 0.89 | 0.68 −− | 1.06 | 0.81 | | ** | |
| uridine-2′,3′-cyclic | 1.03 | 1.59 ++ | 0.87 | 1.34 + | | * | * |
| monophosphate | | | | | | | |
| uracil | 0.91 | 1.54 | 1.01 | 1.71 + | | | |
| 5-methyluridine (ribothymidine) | 1.16 + | 0.99 | 1.22 ++ | 1.04 | ** | | |
| 5,6-dihydrouracil | 1.56 ++ | 2.38 ++ | 0.84 | 1.28 + | | ** | ** |
| 2′-deoxyuridine | 1.54 + | 1.15 + | 1.10 | 0.82 −− | | ** | ** |
| 3-ureidoisobutyrate | 2.27 ++ | 2.41 ++ | 1.21 | 1.28 + | ** | ** | |
| 3-ureidopropionate | 1.54 ++ | 1.71 ++ | 1.04 | 1.15 + | | ** | |
| beta-alanine | 1.12 | 0.95 | 1.18 + | 1.00 | | | |
| 3-(3-amino-3- | 0.97 | 0.89 − | 1.11 | 1.01 | | | |
| carboxypropyl)uridine* | | | | | | | |
| cytidine triphosphate | 1.02 | 0.63 − | 1.21 | 0.75 | | | |
| cytidine diphosphate | 0.95 | 0.6 −− | 1.11 | 0.71 | | * | |
| cytidine 2′,3′-cyclic | 1.03 | 1.31 ++ | 0.91 | 1.15 | | * | * |
| monophosphate | | | | | | | |
| cytidine | 1.06 | 0.93 | 1.22 ++ | 1.07 | ** | | |
| 3-methylcytidine | 0.89 | 0.75 −− | 1.08 | 0.91 | | ** | |
| 5-methylcytidine | 0.96 | 0.77 −− | 1.13 | 0.90 | | ** | |
| 2′-O-methylcytidine | 0.86 −− | 0.92 | 1.06 | 1.14 + | * | ** | |
| 5-methyl-2′-deoxycytidine | 0.75 −− | 0.75 −− | 1.00 | 1.00 | | ** | |
| thymidine | 1.36 + | 1.12 | 1.10 | 0.91 | | ** | * |
| 5,6-dihydrothymine | 1.59 ++ | 1.36 ++ | 0.96 | 0.82 − | | ** | |
| 3-aminoisobutyrate | 0.6 −− | 0.51 −− | 1.09 | 0.93 | | ** | |
| quinolinate | 1.73 ++ | 1.88 ++ | 0.92 | 1.00 | | ** | |
| nicotinamide ribonucleotide | 1.49 + | 1.32 | 1.38 + | 1.23 | ** | ** | |
| (NMN) | | | | | | | |
| 1-methylnicotinamide | 1.81 ++ | 1.85 ++ | 1.02 | 1.04 | | ** | |
| trigonelline (N′- | 0.88 | 1.62 ++ | 0.6 −− | 1.10 | * | * | ** |
| methylnicotinate) | | | | | | | |
| N1-Methyl-2-pyridone-5- | 1.05 | 1.42 ++ | 0.99 | 1.34 ++ | * | ** | * |
| carboxamide | | | | | | | |
| N1-Methyl-4-pyridone-3- | 0.90 | 1.34 ++ | 1.00 | 1.48 ++ | ** | | ** |
| carboxamide | | | | | | | |
| flavin mononucleotide (FMN) | 0.98 | 0.87 − | 1.04 | 0.92 | | | |
| pantothenate | 1.14 + | 1.04 | 1.11 ++ | 1.01 | * | ** | |
| pantetheine | 1.01 | 1.29 + | 0.91 | 1.16 | | | |
| 3′-dephosphocoenzyme A | 1.31 + | 1.17 | 1.08 | 0.96 | | ** | |
| 3′-dephospho-acetyl-CoA | 1.98 + | 1.59 | 0.91 | 0.73 | | * | |
| coenzyme A | 1.18 | 0.82 | 1.05 | 0.73 −− | | | * |
| ascorbic acid 2-sulfate | 1.22 | 1.4 ++ | 1.13 | 1.3 ++ | ** | ** | |
| alpha-tocopherol | 1.02 | 0.99 | 1.29 ++ | 1.24 ++ | ** | | |
| biotin | 1.79 + | 1.17 | 1.08 | 0.7 − | | ** | |
| 5-methyltetrahydrofolate | 0.95 | 1.25 + | 0.77 −− | 1.01 | | | |
| (5MeTHF) | | | | | | | |
| heme | 0.97 | 0.69 −− | 1.06 | 0.76 − | | | |
| bilirubin (Z,Z) | 0.85 | 0.71 −− | 1.06 | 0.88 | | ** | |
| biliverdin | 1.09 | 0.86 | 1.32 + | 1.03 | | | |
| retinol (Vitamin A) | 1.59 + | 1.19 + | 1.14 | 0.85 − | | ** | ** |
| pyridoxamine phosphate | 0.91 | 1.01 | 0.88 − | 0.97 | | | |
| pyridoxal phosphate | 0.71 −− | 1.05 | 1.02 | 1.5 ++ | ** | ** | ** |
| pyridoxal | 1.28 | 0.84 | 1.54 ++ | 1.01 | | | * |
| 3-formylindole | 1.56 ++ | 1.59 ++ | 0.95 | 0.96 | | ** | |
| ergothioneine | 0.68 −− | 0.77 | 1.14 | 1.29 | | ** | |
| erythritol | 1.15 + | 1.07 | 1.07 | 0.99 | | ** | |
| mannonate* | 1.03 | 0.77 −− | 1.27 ++ | 0.95 | | * | ** |
| N-oxalyl glycine (NOG) | 2.77 + | 1.31 | 1.29 | 0.61 | | ** | |
| O-sulfo-L-tyrosine | 1.18 + | 0.97 | 1.10 | 0.90 | | | * |
| S-(3-hydroxypropyl)mercapturic | 1.68 ++ | 1.66 ++ | 1.00 | 0.99 | | ** | |
| acid (HPMA) | | | | | | | |
| perfluorooctanesulfonate (PFOS) | 1.39 + | 1.06 | 1.54 ++ | 1.18 | ** | | |
| triethanolamine | 0.87 | 1.2 + | 0.89 | 1.23 + | | | * |
| thioproline | 0.59 −− | 0.95 | 0.84 | 1.35 | | * | |
| dibutyl sulfosuccinate | 0.7 −− | 0.58 −− | 2.04 ++ | 1.69 ++ | ** | ** | |
| glutamine_degradant* | 1.33 | 1.12 | 0.86 | 0.72 − | * | | |

SPF, specific pathogen free;
CD control diet;
PR, protein restricted;
ABX, antibiotic
“++” indicates that mean values are significantly (p < 0.05) higher for that comparison.
“+” indicates that mean values are significantly (0.05 < p < 0.1) higher for that comparison.
“−−” indicates that mean values are significantly (p < 0.05) lower for that comparison.
“−” indicates that mean values are significantly (0.05 < p < 0.1) lower for that comparison.

TABLE 4
Untargeted metabolomics of maternal serum
Table of statistically significant biochemicals profiled in maternal
serum collected from SPF CD, SPF PR, ABX CD, and ABX PR groups.

| | Fold Change | | | | | | |
|---|---|---|---|---|---|---|---|
| | | ABX PR | ABX CD | ABX PR | ANOVA Main Effects | | |
| | SPF PR vs. | vs. ABX | vs. SPF | vs. SPF | | | Treatment:Diet |
| Biochemical Name | SPF CD | CD | CD | PR | Treatment | Diet | interaction |
| N-acetylglycine | 1.99 ++ | 1.71 ++ | 1.08 | 1.22 | | ** | |
| dimethylglycine | 1.23 ++ | 1.96 ++ | 0.98 | 0.97 | | ** | |
| betaine | 2.47 ++ | 1.22 | 1.15 | 0.68 −− | | ** | ** |
| serine | 0.77 − | 1.11 | 0.76 −− | 1.10 | | | * |
| N-acetylserine | 1.01 | 1.19 ++ | 1.00 | 1.18 ++ | | * | |
| threonine | 0.31 −− | 0.42 −− | 0.92 | 1.24 | | ** | |
| N-acetylthreonine | 0.76 −− | 0.81 − | 1.10 | 1.17 | | ** | |
| allo-threonine | 0.57 | 0.3 −− | 1.85 ++ | 0.96 | | ** | * |
| alanine | 0.77 −− | 1.22 | 0.86 | 1.36 ++ | | | ** |
| N-acetylalanine | 1.33 + | 1.24 ++ | 1.07 | 1.12 | | ** | |
| N,N-dimethylalanine | 1.47 | 2.27 + | 0.95 | 1.46 | | ** | |
| asparagine | 0.6 −− | 0.95 | 0.86 | 1.35 | | * | |
| N-acetylasparagine | 0.56 −− | 0.64 − | 1.14 | 1.31 | | ** | |
| hydroxyasparagine** | 1.11 | 1.28 ++ | 1.04 | 1.2 + | | ** | |
| glutamate | 0.71 − | 0.98 | 0.77 | 1.06 | | | |
| alpha-ketoglutaramate* | 0.87 | 1.79 ++ | 0.35 −− | 0.71 | ** | | |
| N-acetylglutamate | 0.56 −− | 0.76 | 0.82 | 1.11 | | ** | |
| 4-hydroxyglutamate | 0.66 −− | 1.38 | 0.62 −− | 1.29 | | | ** |
| beta-citrylglutamate | 0.62 − | 1.28 | 0.64 | 1.31 | | | * |
| S-1-pyrroline-5-carboxylate | 0.58 −− | 0.95 | 0.66 −− | 1.07 | | * | * |
| histidine | 0.52 −− | 0.72 − | 0.80 | 1.10 | | ** | |
| 1-methylhistidine | 0.56 −− | 0.95 | 0.97 | 1.65 ++ | | ** | * |
| 3-methylhistidine | 1.47 + | 1.9 ++ | 0.88 | 1.26 | | ** | |
| N-acetylhistidine | 0.33 −− | 0.4 −− | 1.08 | 1.3 + | | ** | |
| trans-urocanate | 0.54 | 1.09 | 0.43 −− | 0.87 | * | | |
| imidazole propionate | 0.32 −− | 0.29 − | 0.08 −− | 0.08 −− | ** | ** | |
| imidazole lactate | 0.46 −− | 0.57 − | 1.22 | 1.51 ++ | ** | ** | |
| anserine | 0.85 | 1.97 ++ | 0.58 − | 1.35 | | | * |
| histamine | 0.55 −− | 1.05 | 0.82 | 1.57 ++ | | ** | ** |
| 1-methylhistamine | 0.65 − | 1.12 | 0.84 | 1.44 + | | | * |
| 1-methyl-4-imidazoleacetate | 0.64 −− | 1.04 | 1.00 | 1.63 ++ | * | | * |
| 1-methyl-5-imidazoleacetate | 1.22 + | 2.32 ++ | 0.59 −− | 0.88 | * | ** | * |
| 1-methyl-5-imidazolelactate | 3.83 ++ | 2.13 ++ | 1.26 | 1.42 | ** | ** | |
| 4-imidazoleacetate | 0.47 −− | 1.00 | 0.78 | 1.64 ++ | | ** | ** |
| N-acetylhistamine | 0.45 −− | 0.81 | 0.63 − | 1.14 | | ** | * |
| lysine | 0.87 −− | 1.11 + | 0.81 −− | 1.04 | ** | | ** |
| N6-acetyllysine | 0.93 | 1.19 | 1.02 | 1.3 + | | | |
| N2-acetyl,N6,N6-dimethyllysine | 2.46 ++ | 1.09 | 1.99 + | 0.85 | | ** | * |
| N2,N6-diacetyllysine | 1.02 | 0.88 | 1.29 ++ | 1.11 | ** | | |
| N6-methyllysine | 1.14 | 1.68 ++ | 0.82 −− | 1.21 ++ | | ** | ** |
| N6,N6-dimethyllysine | 1.35 ++ | 1.44 ++ | 0.95 | 1.10 | | ** | |
| hydroxy-N6,N6,N6- | 1.02 | 1.49 ++ | 0.82 | 1.19 | | ** | ** |
| trimethyllysine* | | | | | | | |
| fructosyllysine | 0.69 −− | 0.90 | 0.96 | 1.25 + | | ** | |
| 2-aminoadipate | 0.36 −− | 0.38 −− | 0.87 | 0.93 | | ** | |
| 2-oxoadipate | 0.23 −− | 0.29 −− | 0.94 | 1.21 | | ** | |
| glutarylcarnitine (C5-DC) | 1.73 ++ | 1.35 | 1.41 | 1.13 | | * | |
| pipecolate | 0.89 | 1.62 ++ | 1.03 | 1.88 ++ | ** | ** | ** |
| 6-oxopiperidine-2-carboxylate | 0.62 −− | 0.69 −− | 1.04 | 1.15 | | ** | |
| 5-aminovalerate | 1.44 ++ | 1.94 | 0.26 −− | 0.13 −− | ** | ** | |
| N,N-dimethyl-5-aminovalerate | 0.80 | 1.12 | 1.03 | 1.44 ++ | * | | |
| N,N,N-trimethyl-5- | 1.49 ++ | 1.08 | 0.93 | 0.44 −− | ** | ** | ** |
| aminovalerate | | | | | | | |
| N-acetyl-2-aminoadipate | 0.42 −− | 0.27 −− | 1.17 | 0.75 | | ** | |
| phenylalanine | 0.71 −− | 0.90 | 0.82 − | 1.05 | | ** | * |
| N-acetylphenylalanine | 0.52 −− | 0.5 −− | 1.14 | 1.11 | | ** | |
| phenylpyruvate | 0.55 −− | 0.80 | 0.85 | 1.23 | | ** | |
| phenyllactate (PLA) | 0.38 −− | 1.02 | 0.52 − | 1.41 | | ** | ** |
| phenylacetate | 0.68 −− | 1.00 | 0.28 −− | 0.41 | ** | ** | ** |
| 2-hydroxyphenylacetate | 0.59 −− | 0.66 −− | 1.18 | 1.32 | * | ** | |
| 4-hydroxyphenylacetate | 0.51 −− | 1.19 | 0.3 −− | 0.70 | ** | | ** |
| tyrosine | 0.51 −− | 0.61 −− | 0.70 | 0.83 | | ** | |
| N-acetyltyrosine | 0.54 | 0.43 −− | 0.83 | 0.65 | | ** | |
| 4-hydroxyphenylpyruvate | 0.39 −− | 0.77 | 0.43 − | 0.84 | | ** | |
| 3-(4-hydroxyphenyl)lactate | 0.72 −− | 0.88 | 0.84 | 1.01 | | ** | |
| phenol sulfate | 1.68 ++ | 1.17 | 0.15 −− | 0.06 −− | ** | * | |
| p-cresol glucuronide* | 3.07 | 1.00 | 0.19 −− | 0.06 −− | ** | | |
| 4-hydroxyphenylacetate sulfate | 0.51 −− | 0.6 −− | 0.18 −− | 0.21 −− | ** | ** | |
| thyroxine | 1.85 + | 1.01 | 1.07 | 0.76 | | | |
| tryptophan | 0.49 −− | 0.72 − | 0.85 | 1.26 + | | ** | * |
| N-acetyltryptophan | 0.59 −− | 0.43 −− | 1.45 + | 1.06 | | ** | |
| oxindolylalanine | 0.38 −− | 0.73 | 0.96 | 1.84 ++ | * | ** | * |
| kynurenine | 0.4 −− | 1.11 | 0.65 −− | 1.81 ++ | | ** | ** |
| N-acetylkynurenine (2) | 0.75 | 0.72 − | 1.37 | 1.31 | | ** | |
| kynurenate | 0.59 −− | 1.01 | 0.97 | 1.66 ++ | * | ** | * |
| N-formylanthranilic acid | 0.46 −− | 0.53 −− | 0.58 − | 0.66 | ** | ** | |
| anthranilate | 0.53 −− | 0.69 | 0.55 −− | 0.72 | ** | ** | |
| xanthurenate | 0.11 −− | 0.28 −− | 0.91 | 2.3 + | | ** | |
| picolinate | 0.24 −− | 0.66 −− | 0.68 −− | 1.89 ++ | | ** | ** |
| 5-hydroxyindoleacetate | 0.72 −− | 0.92 | 1.02 | 1.31 ++ | ** | ** | * |
| indolelactate | 0.47 −− | 1.12 | 0.80 | 1.93 ++ | ** | ** | ** |
| indoleacetate | 0.52 −− | 0.85 | 0.91 | 1.48 ++ | | ** | ** |
| indoleacrylate | 0.37 − | 1.00 | 0.06 −− | 0.15 −− | ** | | |
| indolepropionate | 0.68 | 1.00 | 0.02 −− | 0.03 | ** | | |
| indole-3-carboxylate | 0.79 −− | 0.75 −− | 1.02 | 0.96 | | ** | |
| indoleacetylglycine | 0.62 −− | 0.83 | 0.96 | 1.29 | | ** | |
| 3-indoxyl sulfate | 0.55 −− | 0.38 −− | 0.01 −− | 0 −− | ** | ** | |
| leucine | 0.53 −− | 0.74 −− | 0.82 | 1.16 | | ** | * |
| N-acetylleucine | 0.28 −− | 0.41 −− | 1.10 | 1.61 ++ | ** | ** | |
| 4-methyl-2-oxopentanoate | 0.5 −− | 0.75 −− | 0.87 | 1.32 | | ** | |
| alpha-hydroxyisocaproate | 0.39 −− | 1.17 | 0.56 −− | 1.67 ++ | | ** | ** |
| isovalerate (15:0) | 0.37 −− | 0.86 | 0.72 | 1.68 | | ** | |
| isovalerylglycine | 0.41 −− | 1.02 | 0.70 | 1.75 + | | ** | ** |
| 3-methylcrotonylglycine | 0.52 −− | 0.88 | 0.91 | 1.54 + | | ** | |
| beta-hydroxyisovalerate | 0.63 −− | 1.04 | 0.84 | 1.39 ++ | | * | ** |
| beta-hydroxyisovaleroylcarnitine | 0.65 −− | 0.52 −− | 1.08 | 0.86 | | ** | |
| 3-methylglutaconate | 0.48 −− | 0.63 −− | 0.88 | 1.15 | | ** | |
| isoleucine | 0.58 −− | 0.67 −− | 0.95 | 1.10 | | ** | |
| N-acetylisoleucine | 0.47 −− | 0.56 −− | 1.09 | 1.29 | | ** | |
| 3-methyl-2-oxovalerate | 0.5 − | 0.65 −− | 0.95 | 1.25 | | ** | |
| 2-hydroxy-3-methylvalerate | 0.31 −− | 1.16 | 0.74 | 2.8 ++ | ** | ** | ** |
| tigloylglycine | 0.73 | 0.68 − | 1.20 | 1.12 | | * | |
| 3-hydroxy-2-ethylpropionate | 0.84 | 0.93 | 1.52 ++ | 1.69 ++ | ** | | |
| valine | 0.44 −− | 0.64 −− | 0.81 | 1.20 | | ** | ** |
| N-acetylvaline | 0.6 −− | 0.72 −− | 1.00 | 1.20 | | ** | |
| 3-methyl-2-oxobutyrate | 0.73 − | 0.7 −− | 1.34 + | 1.30 | ** | ** | |
| alpha-hydroxyisovalerate | 0.59 −− | 1.07 | 0.98 | 1.79 ++ | ** | ** | ** |
| 3-hydroxyisobutyrate | 0.36 −− | 0.73 − | 0.90 | 1.82 ++ | ** | ** | ** |
| 2,3-dihydroxy-2-methylbutyrate | 1.77 + | 1.99 ++ | 1.04 | 1.41 + | | ** | |
| methionine | 0.37 −− | 0.65 −− | 0.71 −− | 1.25 + | | ** | ** |
| N-acetylmethionine | 0.46 −− | 0.44 −− | 1.19 | 1.15 | | ** | |
| N-formylmethionine | 0.79 −− | 1.02 | 0.78 −− | 1.01 | | | * |
| S-methylmethionine | 1.35 | 2 ++ | 0.53 −− | 0.79 | ** | ** | |
| methionine sulfone | 2.62 ++ | 2.16 ++ | 1.59 ++ | 2.32 ++ | ** | ** | ** |
| methionine sulfoxide | 0.42 −− | 0.75 − | 0.72 −− | 1.29 + | | ** | ** |
| N-acetylmethionine sulfoxide | 0.73 −− | 0.58 −− | 1.22 | 0.97 | | ** | |
| 5-methylthioribose** | 1.69 ++ | 1.26 + | 1.13 | 1.06 | | ** | |
| 2,3-dihydroxy-5-methylthio-4- | 0.88 −− | 0.97 | 1.01 | 1.11 + | | ** | |
| pentenoate (DMTPA)* | | | | | | | |
| 2-hydroxy-4- | 0.28 −− | 0.55 −− | 0.68 −− | 1.33 ++ | | ** | ** |
| (methylthio)butanoic acid | | | | | | | |
| cystathionine | 0.51 −− | 1.13 | 0.7 −− | 1.55 ++ | | ** | ** |
| alpha-ketobutyrate | 0.93 | 0.71 −− | 1.29 ++ | 0.99 | ** | ** | ** |
| cysteine | 0.65 − | 1.00 | 0.91 | 1.40 | | | |
| S-methylcysteine | 1.19 ++ | 2.43 ++ | 0.72 − | 1.04 | | ** | |
| S-methylcysteine sulfoxide | 1.41 ++ | 3.79 ++ | 0.81 | 1.24 | | ** | * |
| cysteine s-sulfate | 0.48 − | 0.37 −− | 1.91 | 1.49 | ** | ** | |
| cystine | 0.39 −− | 1.03 | 0.52 −− | 1.38 | | ** | ** |
| cysteine sulfinic acid | 0.27 −− | 0.53 −− | 0.63 | 1.21 | | ** | |
| hypotaurine | 1.85 + | 0.24 −− | 0.89 | 0.12 | | ** | |
| taurocyamine | 1.37 ++ | 1.56 ++ | 1.44 ++ | 1.56 ++ | ** | ** | |
| arginine | 1.35 ++ | 0.80 | 1.6 ++ | 0.92 | | | ** |
| urea | 0.18 −− | 0.2 −− | 0.98 | 1.13 | | ** | |
| ornithine | 0.58 −− | 1.03 | 0.47 −− | 0.83 | ** | ** | ** |
| 2-oxoarginine* | 0.89 | 0.75 | 1.10 | 0.92 | | ** | |
| homoarginine | 0.99 | 1.38 ++ | 0.76 −− | 1.06 | | ** | ** |
| homocitrulline | 0.64 −− | 1.20 | 1.07 | 1.99 ++ | ** | | ** |
| proline | 0.48 −− | 0.93 | 0.68 −− | 1.31 | | ** | ** |
| dimethylarginine (SDMA + ADMA) | 0.94 | 1.15 + | 0.88 − | 1.08 | | | * |
| N-acetylarginine | 0.71 | 0.65 − | 1.08 | 0.97 | | ** | |
| N-acetylcitrulline | 0.81 − | 0.57 −− | 1.36 ++ | 0.96 | | ** | * |
| N-acetylproline | 0.59 −− | 0.94 | 0.86 | 1.37 | | | |
| N-delta-acetylornithine | 0.80 | 1.31 | 0.61 −− | 1.01 | | | |
| N-alpha-acetylornithine | 0.99 | 1.68 ++ | 0.81 | 1.38 | | ** | |
| N2,N5-diacetylornithine | 0.72 | 0.61 − | 1.46 | 1.24 | | * | |
| trans-4-hydroxyproline | 1.08 | 1.67 ++ | 0.78 | 1.21 | | ** | ** |
| pro-hydroxy-pro | 3 + | 1.28 + | 1.25 | 1.22 | ** | ** | |
| N-methylproline | 1.17 | 0.76 | 1.13 | 0.73 −− | | | * |
| N,N,N-trimethyl-alanylproline | 1.41 | 2.17 ++ | 0.79 | 1.22 | | ** | |
| betaine (TMAP) | | | | | | | |
| argininate* | 0.56 −− | 0.46 −− | 1.76 ++ | 1.45 | ** | ** | |
| N-acetylhomocitrulline | 0.22 −− | 0.47 −− | 0.97 | 2.07 ++ | ** | ** | ** |
| guanidinoacetate | 1.40 | 0.4 −− | 1.19 | 0.34 −− | | ** | ** |
| creatine | 0.92 | 1.48 ++ | 0.71 − | 1.15 | | | * |
| creatinine | 17.63 ++ | 1.34 ++ | 0.97 | 1.09 | | ** | |
| creatine phosphate | 1.14 | 0.84 | 2.71 ++ | 1.99 − | ** | | |
| putrescine | 1.18 | 1.68 ++ | 0.80 | 1.13 | | * | |
| N-acetylputrescine | 1.09 | 1.39 − | 0.98 | 1.25 | | * | |
| spermidine | 1.11 | 1.77 ++ | 0.80 | 1.29 | | * | |
| (N(1) + N(8))-acetylspermidine | 2.14 | 2.44 + | 1.27 | 1.45 | * | ** | |
| spermine | 0.99 | 2.24 + | 0.65 | 1.48 | | | |
| 5-methylthioadenosine (MTA) | 0.64 − | 0.73 | 0.94 | 1.07 | | ** | |
| 4-acetamidobutanoate | 1.17 | 1.31 ++ | 1.13 | 1.27 | ** | ** | |
| 1-methylguanidine | 1.03 | 1.59 ++ | 1.22 | 1.87 ++ | ** | ** | * |
| glutathione, oxidized (GSSG) | 2.5 ++ | 11.1 ++ | 1.87 | 1.18 | | ** | |
| cysteine-glutathione disulfide | 1.47 + | 2.24 ++ | 1.27 | 1.54 | | ** | |
| S-methylglutathione | 1.46 ++ | 1.46 | 1.10 | 0.65 − | | ** | |
| 5-oxoproline | 0.90 | 1.16 | 0.54 −− | 0.70 | ** | | |
| 2-aminobutyrate | 0.53 −− | 0.7 − | 0.88 | 1.16 | | ** | |
| 2-hydroxybutyrate/2- | 0.83 | 0.88 | 1.31 | 1.37 + | ** | | |
| hydroxyisobutyrate | | | | | | | |
| ophthalmate | 1.46 ++ | 1.11 | 1.36 | 0.87 | | * | |
| gamma-glutamylalanine | 0.75 | 0.41 − | 1.44 | 0.79 | | ** | |
| gamma-glutamylglutamate | 0.57 | 0.36 − | 0.30 | 0.19 | ** | ** | |
| gamma-glutamylglutamine | 0.64 | 0.56 −− | 0.76 | 0.67 | | ** | |
| gamma-glutamylglycine | 1.47 | 0.74 | 0.66 | 0.33 −− | ** | | |
| gamma-glutamylhistidine | 0.47 −− | 0.49 −− | 0.77 | 0.79 | | ** | |
| gamma-glutamylisoleucine* | 0.37 −− | 0.55 −− | 0.58 | 0.87 | | ** | |
| gamma-glutamylleucine | 0.36 −− | 0.55 −− | 0.61 | 0.94 | | ** | |
| gamma-glutamyl-alpha-lysine | 0.67 −− | 0.82 | 0.77 | 0.94 | | ** | |
| gamma-glutamyl-epsilon-lysine | 0.71 − | 1.03 | 0.76 − | 1.10 | | | |
| gamma-glutamylmethionine | 0.14 − | 0.16 −− | 0.94 | 1.02 | | ** | |
| gamma-glutamylphenylalanine | 0.72 − | 0.72 −− | 0.94 | 0.95 | | ** | |
| gamma-glutamylthreonine | 0.29 −− | 0.33 −− | 1.14 | 1.29 | * | ** | |
| gamma-glutamyltryptophan | 0.75 − | 0.66 −− | 1.05 | 0.92 | | ** | |
| gamma-glutamyltyrosine | 0.49 −− | 0.42 −− | 0.62 | 0.53 | | ** | |
| gamma-glutamylvaline | 0.42 −− | 0.54 −− | 0.69 | 0.88 | | ** | |
| gamma-glutamylserine | 0.69 − | 0.87 | 0.69 | 0.88 | * | * | |
| gamma-glutamylcitrulline* | 0.45 | 0.25 −− | 1.08 | 0.60 | | ** | |
| gamma-glutamyl-2-aminobutyrate | 0.4 − | 0.34 | 0.96 | 0.81 | | ** | |
| glycylleucine | 0.65 | 1.04 | 0.35 −− | 0.56 | ** | | |
| prolylglycine | 0.5 −− | 0.58 −− | 1.05 | 1.20 | | ** | |
| valylleucine | 0.23 − | 1.36 | 0.13 −− | 0.75 | ** | | |
| glu-gly-asn-val** | 0.59 | 0.44 | 0.3 − | 0.22 | * | * | |
| phenylacetylglutamine | 0.56 −− | 0.95 | 0.52 −− | 0.88 | ** | ** | ** |
| phenylacetylglycine | 0.66 −− | 1.27 | 0.19 −− | 0.36 −− | ** | | ** |
| N,N-dimethyl-pro-pro | 1.82 + | 1.12 | 1.40 | 1.06 | | ** | |
| 1,5-anhydroglucitol (1,5-AG) | 1.11 | 1.42 ++ | 1.07 | 1.36 | ** | ** | |
| glucose | 0.81 −− | 0.85 | 1.15 | 1.2 + | ** | ** | |
| dihydroxyacetone phosphate (DHAP) | 0.31 − | 0.43 | 1.66 | 2.31 + | ** | ** | |
| 3-phosphoglycerate | 1.16 | 2.06 ++ | 1.03 | 1.82 | | ** | |
| pyruvate | 0.75 − | 0.7 −− | 1.17 | 1.10 | | ** | |
| lactate | 2.31 ++ | 0.89 | 1.23 ++ | 0.81 − | | | ** |
| glycerate | 2.52 ++ | 1.36 ++ | 0.98 | 0.91 | | ** | |
| ribose 1-phosphate | 0.64 − | 4.5 ++ | 0.52 | 3.67 ++ | | | ** |
| sedoheptulose-7-phosphate | 0.48 | 1.58 | 0.78 | 2.6 ++ | | | * |
| ribitol | 1.15 | 1.19 ++ | 0.90 | 0.93 | | ** | |
| xylose | 1.4 ++ | 1.56 + | 1.18 | 0.73 | | ** | |
| arabitol/xylitol | 3.01 ++ | 1.7 ++ | 1.01 | 1.23 | | ** | |
| arabonate/xylonate | 1.46 ++ | 1.28 ++ | 1.13 | 1.15 | * | ** | |
| sedoheptulose | 0.60 | 1.38 | 0.39 −− | 0.90 | ** | | * |
| lyxonate | 0.88 | 0.6 −− | 1.37 + | 0.93 | | ** | |
| sucrose | 0.91 | 10.48 ++ | 0.49 − | 5.64 ++ | | ** | ** |
| fructose | 0.38 −− | 4.44 ++ | 0.32 −− | 3.8 ++ | | | ** |
| mannitol/sorbitol | 0.79 | 1.08 | 1.10 | 1.5 + | | | |
| mannose | 0.93 | 0.56 −− | 1.21 | 0.74 | | ** | * |
| 2-ketogulonate | 1.53 ++ | 2.35 ++ | 0.94 | 0.73 | | ** | |
| galactonate | 1.45 ++ | 1.7 ++ | 0.87 | 0.96 | | ** | |
| glucuronate | 1.92 ++ | 1.20 | 1.22 + | 1.01 | | ** | |
| N-acetyl-glucosamine 1- | 0.99 | 1.74 + | 0.84 | 1.47 + | | | * |
| phosphate | | | | | | | |
| N-acetylneuraminate | 1.09 | 1.36 ++ | 0.86 | 1.07 | | ** | |
| N-acetylglucosaminylasparagine | 1.29 + | 1.05 | 1.9 ++ | 1.33 | ** | | |
| N6-carboxymethyllysine | 0.58 | 0.97 | 1.02 | 1.7 ++ | * | | |
| citrate | 1.26 ++ | 1.42 ++ | 1.19 ++ | 1.23 ++ | ** | ** | |
| aconitate [cis or trans] | 1.68 ++ | 1.2 ++ | 1.21 ++ | 1.15 | ** | ** | |
| isocitrate | 3.77 ++ | 1.42 ++ | 1.31 ++ | 1.10 | ** | ** | |
| isocitric lactone | 1.81 ++ | 1.05 | 1.48 ++ | 1.07 | * | ** | |
| alpha-ketoglutarate | 0.78 | 0.65 − | 1.64 ++ | 1.37 | ** | ** | |
| succinylcarnitine (C4-DC) | 2.51 ++ | 2.66 ++ | 1.06 | 1.46 | | ** | |
| citraconate/glutaconate | 0.96 | 1.25 | 1.09 | 1.42 + | * | | |
| acetylphosphate | 0.42 | 1.23 | 0.70 | 2.04 + | | | |
| phosphate | 2.91 ++ | 1.09 | 1.19 + | 0.92 | | ** | * |
| malonylcarnitine | 1.87 ++ | 2.54 ++ | 0.95 | 0.96 | | ** | |
| malonate | 1.14 | 1.35 ++ | 0.95 | 1.12 | | ** | |
| butyrate/isobutyrate (4:0) | 1.20 | 1.05 | 1.35 + | 1.18 | * | | |
| caproate (6:0) | 1.13 | 0.91 | 1.47 + | 1.18 | * | | |
| (2 or 3)-decenoate (10:1n7 or n8) | 2.22 ++ | 0.66 − | 2.14 ++ | 0.61 − | | | ** |
| 10-undecenoate (11:1n1) | 0.46 −− | 0.09 −− | 2.55 ++ | 0.48 | | ** | ** |
| myristate (14:0) | 1.40 | 0.5 − | 1.62 | 0.58 | | | ** |
| pentadecanoate (15:0) | 1.06 | 0.71 − | 1.17 | 0.78 | | | |
| palmitate (16:0) | 1.07 | 0.58 −− | 1.32 | 0.71 | | | * |
| margarate (17:0) | 1.07 | 0.74 −− | 1.16 | 0.79 | | | * |
| stearate (18:0) | 1.20 | 0.63 −− | 1.24 | 0.65 − | | | * |
| nonadecanoate (19:0) | 1.08 | 0.72 − | 1.14 | 0.76 − | | | * |
| arachidate (20:0) | 2.25 ++ | 1.52 ++ | 0.92 | 0.75 − | | ** | |
| myristoleate (14:1n5) | 2.24 ++ | 0.52 | 2.12 ++ | 0.5 − | | | ** |
| palmitoleate (16:1n7) | 1.04 | 0.25 −− | 1.80 | 0.43 | | | ** |
| 10-heptadecenoate (17:1n7) | 0.97 | 0.45 −− | 1.47 | 0.68 | | * | * |
| eicosenoate (20:1) | 1.62 + | 0.82 | 1.08 | 0.48 −− | | | |
| erucate (22:1n9) | 1.94 ++ | 1.17 | 0.87 | 0.56 −− | | ** | |
| tetradecadienoate (14:2)* | 2.04 ++ | 0.44 −− | 2.62 ++ | 0.52 −− | | | ** |
| eicosapentaenoate (EPA; 20:5n3) | 0.88 | 0.31 −− | 1.81 + | 0.65 | | ** | * |
| heneicosapentaenoate (21:5n3) | 1.18 | 0.25 −− | 2.31 ++ | 0.48 | | ** | ** |
| docosapentaenoate (n3 DPA; 22:5n3) | 1.41 | 0.52 −− | 2.63 ++ | 0.97 | ** | | ** |
| docosahexaenoate (DHA; 22:6n3) | 0.93 | 0.46 −− | 1.6 + | 0.79 | | ** | |
| docosatrienoate (22:3n3) | 0.78 | 0.45 −− | 1.31 | 0.75 | | ** | |
| nisinate (24:6n3) | 0.27 −− | 0.14 −− | 2.25 ++ | 1.17 | ** | ** | |
| hexadecadienoate (16:2n6) | 1.71 ++ | 1.25 | 1.61 + | 0.90 | | ** | |
| linoleate (18:2n6) | 1.36 + | 0.88 | 1.46 | 0.80 | | | |
| linolenate [alpha or gamma; | 1.25 | 0.51 − | 1.95 | 0.80 | | | * |
| (18:3n3 or 6)] | | | | | | | |
| dihomo-linolenate (20:3n3 or n6) | 1.12 | 0.52 −− | 1.8 ++ | 0.83 | | | ** |
| arachidonate (20:4n6) | 0.79 | 0.42 −− | 1.22 | 0.66 | | ** | |
| docosatrienoate (22:3n6)* | 0.48 −− | 0.23 −− | 1.33 | 0.64 | | ** | |
| adrenate (22:4n6) | 0.84 | 0.39 −− | 1.86 ++ | 0.85 | | ** | ** |
| docosapentaenoate (n6 DPA; 22:5n6) | 0.61 − | 0.24 −− | 1.67 | 0.65 | | ** | |
| docosadienoate (22:2n6) | 2.29 ++ | 0.88 | 1.27 | 0.73 | | | * |
| mead acid (20:3n9) | 0.63 | 0.23 −− | 1.60 | 0.59 | | ** | * |
| citronellic acid | 0.53 −− | 0.3 −− | 1.35 | 0.78 | | ** | |
| (14 or 15)-methylpalmitate | 0.96 | 0.56 −− | 1.37 + | 0.79 | | ** | ** |
| (a17:0 or i17:0) | | | | | | | |
| 2-hydroxyadipate | 0.58 −− | 0.53 −− | 0.85 | 0.78 | | ** | |
| maleate | 1.68 ++ | 1.40 | 1.27 | 1.04 | | ** | |
| heptenedioate (C7:1-DC)* | 0.45 −− | 0.3 −− | 1.66 | 1.11 | | ** | |
| decadienedioic acid (C10:2-DC)** | 1.50 | 0.51 − | 3.15 + | 1.07 | | | * |
| dodecenedioate (C12:1-DC)* | 1.47 | 1.92 ++ | 0.97 | 1.26 | | ** | |
| dodecadienoate (12:2)* | 2.21 ++ | 0.61 | 2.18 ++ | 0.65 | | | ** |
| tetradecadienedioate (C14:2-DC)* | 1.63 | 0.35 −− | 1.89 | 0.41 −− | | | ** |
| hexadecanedioate (C16-DC) | 1.57 | 0.36 −− | 1.86 | 0.43 −− | | | ** |
| hexadecenedioate (C16:1-DC)* | 1.75 | 0.29 −− | 1.96 + | 0.32 −− | | * | ** |
| heptadecanedioate (C17-DC) | 1.42 | 0.58 −− | 1.33 | 0.55 −− | | | ** |
| octadecanedioate (C18-DC) | 1.56 | 0.44 −− | 1.59 | 0.45 −− | | | ** |
| octadecenedioate (C18:1-DC) | 1.63 | 0.33 −− | 1.71 | 0.34 −− | | | ** |
| octadecadienedioate (C18:2-DC)* | 1.77 | 0.5 −− | 1.28 | 0.36 −− | * | | ** |
| nonadecanedioate (C19-DC) | 1.08 | 0.52 −− | 0.86 | 0.41 −− | ** | * | * |
| eicosanedioate (C20-DC) | 1.25 | 0.68 − | 1.34 | 0.73 | | | ** |
| 2-aminoheptanoate | 3.25 ++ | 4.89 ++ | 0.78 − | 1.54 ++ | | ** | ** |
| 2-aminooctanoate | 1.35 ++ | 1.31 + | 1.59 + | 1.07 | * | ** | |
| N-acetyl-2-aminooctanoate* | 1.58 ++ | 0.97 | 2.29 ++ | 1.19 | ** | ** | * |
| butyrylcarnitine (C4) | 3.94 ++ | 1.12 | 1.26 | 0.84 | | ** | |
| methylmalonate (MMA) | 0.79 −− | 1.03 | 0.86 −− | 1.12 + | | ** | ** |
| isocaproylglycine | 1.46 | 0.62 | 2.06 + | 0.87 | | | |
| hexanoylglycine | 3.64 ++ | 1.47 | 1.90 | 0.71 | | ** | |
| 4-methylhexanoylglycine | 1.62 | 0.56 − | 1.67 | 0.57 | | | ** |
| trans-2-hexenoylglycine | 12.89 ++ | 1.21 | 1.24 | 0.52 −− | | ** | * |
| cis-3,4- | 1.38 | 0.56 −− | 2.15 ++ | 0.87 | ** | | ** |
| methyleneheptanoylglycine | | | | | | | |
| trans-3,4- | 0.91 | 0.28 −− | 2.17 ++ | 0.66 | | ** | ** |
| methyleneheptanoylglycine | | | | | | | |
| N-octanoylglycine | 5.55 ++ | 1.12 | 2.29 + | 0.71 | | ** | |
| 3-hydroxyoctanoylglycine | 1.44 ++ | 1.77 | 3.2 + | 0.44 | | ** | * |
| 2-butenoylglycine | 1.58 ++ | 1.36 | 2.02 | 0.50 | | ** | |
| picolinoylglycine | 0.42 −− | 0.5 −− | 0.98 | 1.16 | | ** | |
| acetylcarnitine (C2) | 3.01 ++ | 1.45 ++ | 1.21 | 1.11 | | ** | |
| hexanoylcarnitine (C6) | 3.02 ++ | 1.09 | 1.98 ++ | 0.67 − | | ** | ** |
| octanoylcarnitine (C8) | 3.03 ++ | 1.03 | 1.92 ++ | 0.66 − | | ** | ** |
| cis-3,4- | 3.33 ++ | 1.07 | 1.32 | 0.97 | | * | |
| methyleneheptanoylcarnitine | | | | | | | |
| decanoylcarnitine (C10) | 2.99 ++ | 0.73 | 2.1 ++ | 0.43 −− | | * | ** |
| laurylcarnitine (C12) | 1.65 ++ | 0.66 − | 1.93 ++ | 0.57 −− | | | ** |
| myristoylcarnitine (C14) | 1.6 + | 0.57 −− | 1.72 ++ | 0.54 −− | | | ** |
| palmitoylcarnitine (C16) | 1.21 | 0.54 −− | 1.49 + | 0.67 | | * | ** |
| stearoylcarnitine (C18) | 1.86 + | 1.02 | 1.14 | 0.86 | | | |
| cis-4-decenoylcarnitine (C10:1) | 2.82 ++ | 1.20 | 1.62 ++ | 0.64 −− | | ** | ** |
| undecenoylcarnitine (C11:1) | 1.33 | 0.29 −− | 2.09 | 0.46 −− | | ** | ** |
| 5-dodecenoylcarnitine (C12:1) | 1.95 ++ | 0.75 − | 1.37 | 0.62 −− | | | ** |
| myristoleoylcarnitine (C14:1)* | 1.98 ++ | 0.62 −− | 1.75 ++ | 0.55 −− | | | ** |
| palmitoleoylcarnitine (C16:1)* | 1.54 | 0.46 −− | 1.88 ++ | 0.56 − | | | ** |
| oleoylcarnitine (C18:1) | 1.32 | 0.58 −− | 1.61 + | 0.71 | | | ** |
| linoleoylcarnitine (C18:2)* | 2.61 ++ | 0.74 − | 1.92 ++ | 0.72 − | | | ** |
| linolenoylcarnitine (C18:3)* | 1.51 | 0.48 −− | 1.88 . | 0.59 −− | | | ** |
| arachidonoylcarnitine (C20:4) | 1.38 | 0.46 −− | 1.79 ++ | 0.6 −− | | * | ** |
| dihomo-linolenoylcarnitine | 2.27 | 0.41 −− | 3.82 ++ | 0.70 | ** | | ** |
| (C20:3n3 or 6)* | | | | | | | |
| docosahexaenoylcarnitine | 2.73 + | 1.18 | 1.14 | 1.08 | | * | |
| (C22:6)* | | | | | | | |
| adipoylcarnitine (C6-DC) | 1.51 + | 2.58 ++ | 0.72 | 1.02 | | ** | |
| pimeloylcarnitine/3- | 1.30 | 3.69 ++ | 0.56 | 1.57 | | ** | |
| methyladipoylcarnitine (C7-DC) | | | | | | | |
| (R)-3-hydroxybutyrylcarnitine | 4.34 ++ | 1.84 | 1.78 | 0.99 | | * | |
| (S)-3-hydroxybutyrylcarnitine | 1.65 ++ | 1.78 | 1.37 | 0.81 | | ** | |
| 3-hydroxyhexanoylcarnitine (1) | 1.87 ++ | 1.59 + | 1.27 | 1.22 | | ** | |
| 3-hydroxyoctanoylcarnitine (1) | 4.36 ++ | 1.24 | 1.89 ++ | 0.83 | | ** | ** |
| 3-hydroxyoctanoylcarnitine (2) | 1.58 ++ | 1.10 | 1.71 | 1.01 | | ** | |
| 3-hydroxydecanoylcarnitine | 3.52 ++ | 1.13 | 2.41 ++ | 0.63 | | ** | ** |
| 3-hydroxyoleoylcarnitine | 1.42 ++ | 0.97 | 1.8 + | 0.67 | | | ** |
| carnitine | 2.27 ++ | 1.74 ++ | 0.90 | 1.00 | | ** | |
| 4-hydroxybutyrate (GHB) | 3.13 ++ | 0.87 | 2.09 ++ | 0.52 −− | | ** | ** |
| alpha-hydroxycaproate | 0.98 | 0.98 | 1.29 ++ | 1.29 + | ** | | |
| 2-hydroxyoctanoate | 2.1 ++ | 0.80 | 1.6 ++ | 0.90 | | | ** |
| 2-hydroxylaurate | 1.48 | 0.59 − | 1.88 ++ | 0.75 | | | ** |
| 2-hydroxypalmitate | 1.09 | 0.46 −− | 1.45 | 0.61 | | ** | ** |
| 2-hydroxystearate | 1.42 | 0.55 − | 1.38 | 0.54 | | | |
| 3-hydroxyhexanoate | 1.91 | 0.81 | 2.35 + | 1.00 | | | |
| 3-hydroxyoctanoate | 1.98 ++ | 0.51 −− | 2.37 ++ | 0.58 − | | | ** |
| 3-hydroxydecanoate | 2.05 ++ | 0.48 −− | 2.71 | 0.66 | | | ** |
| 3-hydroxysebacate | 1.98 ++ | 1.19 | 1.51 | 0.58 −− | | ** | ** |
| 3-hydroxylaurate | 1.37 ++ | 0.39 −− | 2.93 ++ | 0.55 | | | ** |
| 3-hydroxymyristate | 1.53 + | 0.35 −− | 2.32 ++ | 0.51 −− | | | ** |
| 3-hydroxypalmitate | 1.29 | 0.42 −− | 1.32 | 0.43 | | * | |
| 3-hydroxystearate | 1.34 | 0.53 −− | 1.58 + | 0.62 | | | * |
| 5-hydroxyhexanoate | 1.44 | 1.37 + | 1.16 | 1.11 | | ** | |
| 16-hydroxypalmitate | 1.32 + | 0.54 −− | 1.6 ++ | 0.56 − | | | ** |
| 12,13-DiHOME | 1.18 ++ | 0.85 | 2.02 ++ | 0.57 | | * | ** |
| 9,10-DiHOME | 2.2 ++ | 1.23 | 1.83 + | 1.00 | | ** | |
| 2S,3R-dihydroxybutyrate | 1.03 | 0.96 | 1.5 + | 1.39 | ** | | |
| 2R,3R-dihydroxybutyrate | 0.7 −− | 0.59 −− | 1.68 ++ | 1.4 ++ | ** | ** | |
| 5-HETE | 0.46 −− | 0.65 | 0.38 − | 0.54 | * | ** | |
| 12-HHTrE | 0.53 | 1.73 + | 0.56 −− | 1.83 | | | ** |
| oleoyl ethanolamide | 1.20 | 0.72 −− | 1.14 | 0.68 −− | | | ** |
| stearoyl ethanolamide | 1.78 ++ | 0.82 | 1.31 ++ | 0.79 | | | ** |
| N-myristoyltaurine* | 1.56 | 0.41 −− | 1.78 + | 0.47 − | | | ** |
| N-oleoyltaurine | 1.37 | 0.54 −− | 1.19 | 0.47 −− | * | | ** |
| N-stearoyltaurine | 1.20 | 0.81 − | 1.08 | 0.72 −− | | | * |
| N-palmitoyltaurine | 0.82 − | 0.32 −− | 1.42 | 0.55 | | ** | ** |
| N-linoleoyltaurine* | 1.7 ++ | 0.61 −− | 1.56 ++ | 0.54 −− | | | ** |
| linoleoyl ethanolamide | 1.29 ++ | 0.71 − | 1.43 ++ | 0.59 −− | | | ** |
| choline phosphate | 1.22 | 1.80 | 1.81 ++ | 2.67 ++ | ** | * | |
| phosphoethanolamine | 0.95 | 1.07 | 2.02 + | 2.26 + | ** | | |
| glycerophosphoserine* | 0.85 | 1.08 | 1.06 | 1.33 + | | | |
| trimethylamine N-oxide | 1.71 | 1.56 | 0.01 −− | 0.01 −− | ** | * | |
| 1-myristoyl-2-palmitoyl-GPC | 1.35 | 0.58 | 2.53 ++ | 1.09 | | | * |
| (14:0/16:0) | | | | | | | |
| 1-myristoyl-2-arachidonoyl- | 1.30 | 0.47 −− | 2.64 ++ | 0.95 | ** | | ** |
| GPC (14:0/20:4)* | | | | | | | |
| 1,2-dipalmitoyl-GPC (16:0/16:0) | 1.13 | 0.87 | 1.47 ++ | 1.13 | ** | | |
| 1-palmitoyl-2-palmitoleoyl-GPC | 0.90 | 0.43 −− | 2.03 ++ | 0.97 | ** | ** | ** |
| (16:0/16:1)* | | | | | | | |
| 1-palmitoyl-2-stearoyl-GPC | 1.16 | 0.73 − | 1.65 ++ | 1.04 | ** | | * |
| (16:0/18:0) | | | | | | | |
| 1-palmitoyl-2-oleoyl-GPC | 0.88 | 0.66 −− | 1.18 + | 0.88 | | ** | ** |
| (16:0/18:1) | | | | | | | |
| 1-palmitoyl-2-linoleoyl-GPC | 1.83 ++ | 1.10 | 1.26 ++ | 1.02 | * | ** | |
| (16:0/18:2) | | | | | | | |
| 1-palmitoyl-2-gamma- | 1.15 | 0.51 −− | 2.28 ++ | 1.00 | ** | ** | ** |
| linolenoyl-GPC (16:0/18:3n6)* | | | | | | | |
| 1-palmitoyl-2-dihomo- | 1.25 | 0.6 −− | 1.67 ++ | 0.80 | | | ** |
| linolenoyl-GPC (16:0/20:3n3 or 6)* | | | | | | | |
| 1-palmitoyl-2-arachidonoyl- | 0.92 | 0.66 −− | 1.46 ++ | 1.04 | ** | ** | ** |
| GPC (16:0/20:4n6) | | | | | | | |
| 1-palmitoyl-2-docosahexaenoyl- | 0.76 −− | 0.71 −− | 1.41 ++ | 1.33 ++ | ** | ** | |
| GPC (16:0/22:6) | | | | | | | |
| 1-palmitoleoyl-2-linoleoyl-GPC | 1.29 ++ | 0.75 − | 2.05 ++ | 1.05 | ** | | ** |
| (16:1/18:2)* | | | | | | | |
| 1-palmitoleoy1-2-linolenoyl- | 0.89 | 0.21 −− | 3.54 ++ | 0.82 | ** | ** | ** |
| GPC (16:1/18:3)* | | | | | | | |
| 1-stearoyl-2-oleoyl-GPC | 0.78 | 0.43 −− | 1.57 ++ | 0.86 | | ** | ** |
| (18:0/18:1) | | | | | | | |
| 1-stearoyl-2-linoleoyl-GPC | 1.28 ++ | 0.95 | 1.35 ++ | 0.99 | ** | | ** |
| (18:0/18:2)* | | | | | | | |
| 1-stearoyl-2-arachidonoyl-GPC | 0.83 − | 0.59 −− | 1.43 | 1.02 | ** | ** | ** |
| (18:0/20:4) | | | | | | | |
| 1-stearoyl-2-docosahexaenoyl- | 0.82 | 0.72 −− | 1.49 ++ | 1.32 ++ | ** | ** | |
| GPC (18:0/22:6) | | | | | | | |
| 1-oleoyl-2-docosahexaenoyl- | 0.52 −− | 0.41 −− | 1.51 ++ | 1.19 | ** | ** | |
| GPC (18:1/22:6)* | | | | | | | |
| 1-linoleoyl-2-arachidonoyl-GPC | 0.93 | 0.36 −− | 2.3 ++ | 0.88 | ** | ** | ** |
| (18:2/20:4n6)* | | | | | | | |
| 1,2-dipalmitoyl-GPE | 1.17 | 0.88 | 1.7 ++ | 1.28 | ** | | |
| (16:0/16:0)* | | | | | | | |
| 1-palmitoyl-2-oleoyl-GPE | 1.42 + | 1.04 | 1.42 ++ | 1.14 | ** | | |
| (16:0/18:1) | | | | | | | |
| 1-palmitoyl-2-linoleoyl-GPE | 1.54 ++ | 2.39 ++ | 1.11 | 1.20 | | ** | |
| (16:0/18:2) | | | | | | | |
| 1-palmitoyl-2-arachidonoyl-GPE | 1.12 | 0.88 | 1.58 ++ | 1.25 + | ** | | |
| (16:0/20:4)* | | | | | | | |
| 1-palmitoyl-2-docosahexaenoyl- | 1.22 | 1.38 | 1.55 | 1.76 ++ | ** | | |
| GPE (16:0/22:6)* | | | | | | | |
| 1-stearoyl-2-linoleoyl-GPE | 1.79 ++ | 1.34 | 1.31 | 1.14 | | ** | |
| (18:0/18:2)* | | | | | | | |
| 1-stearoyl-2-arachidonoyl-GPE | 1.11 | 0.93 | 1.48 ++ | 1.23 + | ** | | |
| (18:0/20:4) | | | | | | | |
| 1-stearoyl-2-docosahexaenoyl- | 1.38 | 2.18 ++ | 1.10 | 1.74 ++ | ** | ** | ** |
| GPE (18:0/22:6)* | | | | | | | |
| 1-oleoyl-2-linoleoyl-GPE | 2.1 + | 1.76 ++ | 1.03 | 1.21 | | ** | |
| (18:1/18:2)* | | | | | | | |
| 1-oleoyl-2-arachidonoyl-GPE | 1.04 | 0.89 | 1.48 ++ | 1.27 + | ** | | |
| (18:1/20:4)* | | | | | | | |
| 1-oleoyl-2-docosahexaenoyl- | 0.95 | 1.14 | 1.36 + | 1.63 ++ | ** | | |
| GPE (18:1/22:6)* | | | | | | | |
| 1,2-dilinoleoyl-GPE | 1.48 | 2.34 ++ | 0.95 | 1.50 | | ** | |
| (18:2/18:2)* | | | | | | | |
| 1-linoleoyl-2-arachidonoyl-GPE | 1.28 | 0.94 | 1.81 ++ | 1.32 | ** | | |
| (18:2/20:4)* | | | | | | | |
| 1-palmitoyl-2-linoleoyl-GPI | 1.31 | 0.78 | 1.67 ++ | 1.00 | | | |
| (16:0/18:2) | | | | | | | |
| 1-palmitoyl-2-arachidonoyl-GPI | 0.61 −− | 0.29 −− | 1.99 ++ | 0.95 | ** | ** | ** |
| (16:0/20:4)* | | | | | | | |
| 1-stearoyl-2-arachidonoyl-GPI | 0.74 −− | 0.54 −− | 1.33 ++ | 0.97 | * | ** | |
| (18:0/20:4) | | | | | | | |
| 1-oleoyl-2-arachidonoyl-GPI | 0.38 −− | 0.23 −− | 1.58 | 0.98 | | ** | |
| (18:1/20:4)* | | | | | | | |
| 1-palmitoyl-GPA (16:0) | 0.55 − | 0.70 | 0.71 | 0.91 | | ** | |
| 1-palmitoyl-GPC (16:0) | 0.66 −− | 0.5 −− | 1.19 | 0.91 | | ** | |
| 2-palmitoyl-GPC (16:0)* | 0.57 −− | 0.39 −− | 1.17 | 0.79 | | ** | |
| 1-palmitoleoyl-GPC (16:1)* | 0.44 −− | 0.24 −− | 1.37 | 0.76 | | ** | |
| 2-palmitoleoyl-GPC (16:1)* | 0.5 −− | 0.11 −− | 1.77 + | 0.39 | | ** | ** |
| 1-stearoyl-GPC (18:0) | 0.67 −− | 0.47 −− | 1.23 | 0.86 | | ** | |
| 1-oleoyl-GPC (18:1) | 0.61 −− | 0.33 −− | 1.15 | 0.63 | | ** | |
| 1-linoleoyl-GPC (18:2) | 0.94 | 0.56 −− | 1.26 | 0.75 | | ** | |
| 1-linolenoyl-GPC (18:3)* | 0.61 −− | 0.18 −− | 1.82 ++ | 0.55 − | | ** | ** |
| 1-arachidonoyl-GPC (20:4n6)* | 0.62 −− | 0.29 −− | 1.11 | 0.52 | | ** | |
| 1-lignoceroyl-GPC (24:0) | 1.01 | 0.62 −− | 1.54 ++ | 0.95 | * | ** | * |
| 1-palmitoyl-GPE (16:0) | 0.62 −− | 0.82 − | 1.06 | 1.4 ++ | ** | ** | * |
| 1-stearoyl-GPE (18:0) | 0.77 −− | 0.78 −− | 1.14 | 1.16 | * | ** | |
| 2-stearoyl-GPE (18:0)* | 0.77 − | 0.64 −− | 1.12 | 0.94 | | ** | |
| 1-oleoyl-GPE (18:1) | 0.64 −− | 0.65 −− | 0.97 | 0.98 | | ** | |
| 1-arachidonoyl-GPE (20:4n6)* | 0.67 −− | 0.4 −− | 1.17 | 0.7 − | | ** | * |
| 1-palmitoyl-GPG (16:0)* | 0.5 − | 0.29 −− | 1.17 | 0.67 | | ** | |
| 1-stearoyl-GPG (18:0) | 0.23 −− | 0.23 −− | 0.67 | 0.68 | ** | ** | |
| 1-oleoyl-GPG (18:1)* | 0.68 −− | 0.47 −− | 1.03 | 0.70 | | ** | |
| 1-linoleoyl-GPG (18:2)* | 0.67 −− | 0.31 −− | 1.30 | 0.60 | | ** | |
| 1-palmitoyl-GPI (16:0) | 0.5 −− | 0.34 −− | 0.99 | 0.67 | | ** | |
| 1-stearoyl-GPI (18:0) | 0.61 −− | 0.7 −− | 0.82 | 0.94 | | ** | |
| 1-oleoyl-GPI (18:1) | 0.53 −− | 0.71 | 0.84 | 1.13 | | ** | |
| 1-linoleoyl-GPI (18:2)* | 1.06 | 0.47 −− | 1.19 | 0.53 | | ** | |
| 1-arachidonoyl-GPI (20:4)* | 0.63 −− | 0.26 −− | 1.28 | 0.53 | | ** | |
| 1-(1-enyl-palmitoyl)-2-linoleoyl- | 1.45 | 0.49 − | 1.84 + | 0.62 | | | * |
| GPE (P-16:0/18:2)* | | | | | | | |
| 1-(1-enyl-palmitoyl)-2- | 2.17 ++ | 1.00 | 1.4 ++ | 0.97 | | ** | * |
| palmitoyl-GPC (P-16:0/16:0)* | | | | | | | |
| 1-(1-enyl-palmitoyl)-2-oleoyl- | 1.79 ++ | 0.98 | 1.18 + | 0.90 | | | ** |
| GPC (P-16:0/18:1)* | | | | | | | |
| 1-(1-enyl-stearoyl)-2-linoleoyl- | 1.13 | 0.80 | 1.5 ++ | 1.06 | * | | |
| GPE (P-18:0/18:2)* | | | | | | | |
| 1-(1-enyl-palmitoyl)-2- | 0.93 | 0.65 −− | 1.06 | 0.74 | | ** | |
| arachidonoyl-GPC (P-16:0/20:4)* | | | | | | | |
| 1-(1-enyl-palmitoyl)-2-linoleoyl- | 1.55 | 0.62 − | 1.20 | 0.48 −− | | | ** |
| GPC (P-16:0/18:2)* | | | | | | | |
| 1-(1-enyl-stearoyl)-2- | 1.05 | 0.75 −− | 1.38 ++ | 0.99 | * | | |
| arachidonoyl-GPE (P-18:0/20:4)* | | | | | | | |
| 1-(1-enyl-palmitoyl)-GPC (P-16:0)* | 0.8 − | 0.61 −− | 0.99 | 0.76 | | ** | |
| 1-(1-enyl-palmitoyl)-GPE (P-16:0)* | 0.62 − | 0.78 | 0.96 | 1.20 | | ** | |
| glycerol | 1.52 ++ | 1.07 | 1.48 ++ | 0.80 | | ** | ** |
| glycerol 3-phosphate | 0.93 | 1.05 | 1.13 | 1.28 + | ** | | |
| glycerophosphoglycerol | 0.66 − | 0.66 | 1.01 | 1.02 | | ** | |
| 1-palmitoylglycerol (16:0) | 0.67 −− | 0.82 | 0.95 | 1.16 | | ** | |
| 1-palmitoleoylglycerol (16:1)* | 0.45 −− | 0.4 −− | 1.21 | 1.07 | | ** | |
| 1-linolenoylglycerol (18:3) | 0.78 | 0.66 −− | 1.31 + | 1.09 | * | ** | |
| 1-arachidonylglycerol (20:4) | 0.62 − | 0.64 − | 0.98 | 1.01 | | ** | |
| 1-docosahexaenoylglycerol (22:6) | 0.56 −− | 0.63 −− | 1.47 + | 1.66 ++ | ** | ** | |
| 2-palmitoylglycerol (16:0) | 0.59 −− | 0.91 | 0.94 | 1.45 + | | * | * |
| 2-linoleoylglycerol (18:2) | 1.00 | 0.68 − | 1.47 + | 0.99 | | | |
| 2-docosahexaenoylglycerol (22:6)* | 0.66 | 1.26 | 1.26 | 2.43 ++ | ** | | |
| palmitoleoyl-linoleoyl-glycerol | 0.96 | 0.63 | 2.32 + | 1.51 | | | |
| (16:1/18:2) [1]* | | | | | | | |
| palmitoyl-arachidonoyl-glycerol | 1.25 | 0.89 | 1.76 ++ | 1.26 | * | | |
| (16:0/20:4) [2]* | | | | | | | |
| palmitoleoyl-arachidonoyl- | 1.93 | 1.45 | 2.39 ++ | 1.79 + | ** | | |
| glycerol (16:1/20:4) [2]* | | | | | | | |
| palmitoyl-docosahexaenoyl- | 0.86 | 1.11 | 1.37 | 1.78 ++ | ** | | |
| glycerol (16:0/22:6) [1]* | | | | | | | |
| stearoyl-linoleoyl-glycerol | 1.46 + | 3.8 ++ | 1.00 | 1.81 + | | ** | |
| (18:0/18:2) [1]* | | | | | | | |
| stearoyl-linoleoyl-glycerol | 1.86 ++ | 1.98 | 2.11 | 0.96 | | * | |
| (18:0/18:2) [2]* | | | | | | | |
| oleoyl-oleoyl-glycerol | 0.56 | 0.51 − | 1.05 | 0.96 | | * | |
| (18:1/18:1) [2]* | | | | | | | |
| linoleoyl-linoleoyl-glycerol | 2.27 | 2.65 + | 1.50 | 1.75 | | ** | |
| (18:2/18:2) [1]* | | | | | | | |
| linoleoyl-linoleoyl-glycerol | 2.17 | 2.55 + | 1.38 | 1.62 | | ** | |
| (18:2/18:2) [2]* | | | | | | | |
| linoleoyl-linolenoyl-glycerol | 1.27 + | 1.72 | 2.23 | 1.40 | | * | |
| (18:2/18:3) [1]* | | | | | | | |
| linoleoyl-linolenoyl-glycerol | 1.64 ++ | 2.47 ++ | 2.51 + | 1.64 + | ** | ** | |
| (18:2/18:3) [2] | | | | | | | |
| oleoyl-arachidonoyl-glycerol | 1.06 | 0.68 − | 1.86 ++ | 1.19 | ** | | |
| (18:1/20:4) [1]* | | | | | | | |
| oleoyl-arachidonoyl-glycerol | 1.01 | 0.58 −− | 2.13 ++ | 1.23 | ** | * | * |
| (18:1/20:4) [2]* | | | | | | | |
| linoleoyl-arachidonoyl-glycerol | 1.96 ++ | 1.00 | 2.39 ++ | 1.28 | ** | | |
| (18:2/20:4) [1]* | | | | | | | |
| linoleoyl-arachidonoyl-glycerol | 1.53 ++ | 0.91 | 2.41 ++ | 1.32 + | ** | | * |
| (18:2/20:4) [2]* | | | | | | | |
| sphinganine-1-phosphate | 0.62 −− | 0.5 −− | 1.26 | 1.01 | | ** | |
| N-palmitoyl-phytosphingosine | 1.96 ++ | 1.42 | 1.22 | 0.97 | | ** | |
| (t18:0/16:0) | | | | | | | |
| ceramide (d18:1/20:0, | 1.84 ++ | 1.59 | 1.37 | 1.12 | | ** | |
| d16:1/22:0, d20:1/18:0)* | | | | | | | |
| glycosyl-N-palmitoyl- | 0.91 | 0.84 − | 1.12 | 1.03 | | * | |
| sphingosine (d18:1/16:0) | | | | | | | |
| glycosyl-N-stearoyl-sphingosine | 8.58 ++ | 1.47 ++ | 1.32 ++ | 1.06 | * | ** | |
| (d18:1/18:0) | | | | | | | |
| glycosyl ceramide (d18:1/20:0, | 2.85 ++ | 1.47 ++ | 1.69 ++ | 0.94 | ** | ** | ** |
| d16:1/22:0)* | | | | | | | |
| glycosyl ceramide (d18:1/23:1, | 1.42 ++ | 0.82 | 1.44 + | 0.66 −− | | | ** |
| d17:1/24:1) | | | | | | | |
| glycosyl ceramide (d18:2/24:1, | 1.68 ++ | 1.06 | 1.27 ++ | 0.88 | | ** | ** |
| d18:1/24:2)* | | | | | | | |
| lactosyl-N-palmitoyl- | 1.50 | 0.88 | 1.74 + | 1.03 | | | |
| sphingosine (d18:1/16:0) | | | | | | | |
| palmitoyl dihydrosphingomyelin | 1.13 ++ | 0.84 −− | 1.47 ++ | 0.97 | ** | | ** |
| (d18:0/16:0)* | | | | | | | |
| behenoyl dihydrosphingomyelin | 3.55 ++ | 0.81 | 3.42 ++ | 0.88 | ** | * | ** |
| (d18:0/22:0)* | | | | | | | |
| sphingomyelin (d18:0/18:0, | 2 ++ | 1.31 ++ | 1.88 ++ | 0.87 | ** | ** | ** |
| d19:0/17:0)* | | | | | | | |
| palmitoyl sphingomyelin | 1.57 ++ | 0.98 | 1.18 ++ | 1.02 | ** | | ** |
| (d18:1/16:0) | | | | | | | |
| hydroxypalmitoyl sphingomyelin | 1.49 + | 1.14 | 0.95 | 0.85 | | * | |
| (d18:1/16:0(OH))** | | | | | | | |
| stearoyl sphingomyelin | 2.99 ++ | 1.73 ++ | 1.11 | 1.05 | | ** | |
| (d18:1/18:0) | | | | | | | |
| behenoyl sphingomyelin | 1.63 ++ | 1.00 | 1.94 ++ | 0.97 | ** | ** | ** |
| (d18:1/22:0)* | | | | | | | |
| tricosanoyl sphingomyelin | 1.43 ++ | 0.75 −− | 1.95 ++ | 0.93 | ** | | ** |
| (d18:1/23:0)* | | | | | | | |
| lignoceroyl sphingomyelin | 1.52 ++ | 0.91 | 1.79 ++ | 1.00 | ** | ** | ** |
| (d18:1/24:0) | | | | | | | |
| sphingomyelin (d18:2/18:1)* | 1.89 ++ | 0.65 −− | 1.51 ++ | 0.69 −− | | | ** |
| sphingomyelin (d18:2/23:1)* | 1.38 ++ | 0.68 −− | 2 ++ | 0.71 − | * | | ** |
| sphingomyelin (d18:2/24:2)* | 1.68 ++ | 1.11 | 1.34 ++ | 0.89 | | ** | ** |
| sphingomyelin (d18:1/14:0, | 0.77 | 0.46 −− | 1.64 ++ | 0.98 | ** | ** | ** |
| d16:1/16:0)* | | | | | | | |
| sphingomyelin (d18:2/14:0, | 0.97 | 0.64 −− | 1.51 ++ | 1.00 | ** | ** | ** |
| d18:1/14:1)* | | | | | | | |
| sphingomyelin (d17:1/16:0, | 1.02 | 0.67 −− | 1.54 ++ | 1.01 | ** | ** | ** |
| d18:1/15:0, d16:1/17:0)* | | | | | | | |
| sphingomyelin (d18:2/16:0, | 1.46 ++ | 0.71 −− | 1.35 ++ | 0.81 −− | | * | ** |
| d18:1/16:1)* | | | | | | | |
| sphingomyelin (d18:1/17:0, | 2.13 ++ | 0.99 | 1.5 ++ | 0.97 | ** | ** | ** |
| d17:1/18:0, d19:1/16:0) | | | | | | | |
| sphingomyelin (d18:1/18:1, | 1.84 ++ | 1.11 | 1.11 | 0.73 −− | | ** | ** |
| d18:2/18:0) | | | | | | | |
| sphingomyelin (d18:1/19:0, | 2.29 ++ | 1.18 | 1.78 ++ | 0.99 | ** | ** | ** |
| d19:1/18:0)* | | | | | | | |
| sphingomyelin (d18:1/20:0, | 2.12 ++ | 1.24 ++ | 1.79 ++ | 0.96 | ** | ** | ** |
| d16:1/22:0)* | | | | | | | |
| sphingomyelin (d18:1/20:1, | 2.36 ++ | 1.49 ++ | 1.14 | 0.78 | | ** | * |
| d18:2/20:0)* | | | | | | | |
| sphingomyelin (d18:1/21:0, | 1.4 ++ | 0.82 | 2.31 ++ | 0.89 | ** | ** | ** |
| d17:1/22:0, d16:1/23:0)* | | | | | | | |
| sphingomyelin (d18:1/22:1, | 1.69 ++ | 0.86 | 1.6 ++ | 0.75 −− | | ** | ** |
| d18:2/22:0, d16:1/24:1)* | | | | | | | |
| sphingomyelin (d18:1/22:2, | 1.57 ++ | 1.14 | 1.4 ++ | 0.95 | | ** | ** |
| d18:2/22:1, d16:1/24:2)* | | | | | | | |
| sphingomyelin (d18:2/23:0, | 1.32 ++ | 0.74 −− | 1.57 ++ | 0.81 −− | ** | | ** |
| d18:1/23:1, d17:1/24:1)* | | | | | | | |
| sphingomyelin (d18:1/24:1, | 3.54 ++ | 0.97 | 1.4 ++ | 0.97 | ** | ** | ** |
| d18:2/24:0)* | | | | | | | |
| sphingomyelin (d18:2/24:1, | 2.01 ++ | 0.99 | 1.35 ++ | 0.85 | | ** | ** |
| d18:1/24:2)* | | | | | | | |
| palmitoyl-sphingosine- | 2.99 ++ | 4.2 ++ | 0.97 | 1.36 | | ** | |
| phosphoethanolamine (d18:1/16:0) | | | | | | | |
| sphingosine 1-phosphate | 0.84 | 0.69 −− | 1.09 | 0.90 | | ** | |
| mevalonate | 1.07 | 0.75 − | 1.10 | 0.77 | | | |
| cholesterol | 2.35 ++ | 0.75 −− | 1.75 ++ | 1.00 | ** | | ** |
| cholesterol sulfate | 1.37 + | 0.59 −− | 2.27 ++ | 1.10 | ** | ** | ** |
| 7-alpha-hydroxy-3-oxo-4- | 1.01 | 0.81 − | 1.00 | 0.8 − | | | |
| cholestenoate (7-Hoca) | | | | | | | |
| 4-cholesten-3-one | 3.75 ++ | 2 ++ | 1.28 | 0.86 | | ** | |
| campesterol | 2.44 ++ | 0.94 | 1.67 ++ | 0.78 − | | ** | ** |
| corticosterone | 1.23 | 1.68 ++ | 1.03 | 1.41 ++ | * | ** | * |
| androsterone sulfate | 1.00 | 0.4 −− | 1.31 | 0.52 | | ** | * |
| cholate | 4.3 ++ | 0.90 | 0.01 −− | 0 −− | ** | ** | ** |
| glycocholate | 2.91 ++ | 1.00 | 0.37 | 0.09 −− | ** | ** | ** |
| taurocholate | 4.32 ++ | 1.34 | 1.82 + | 0.65 | | ** | |
| chenodeoxycholate | 2.35 ++ | 1.00 | 0.16 −− | 0.05 −− | ** | ** | ** |
| taurochenodeoxycholate | 1.52 ++ | 2.47 + | 2.54 ++ | 0.73 | * | ** | |
| beta-muricholate | 1.19 + | 2.54 + | 0.01 −− | 0.01 −− | ** | ** | |
| tauro-beta-muricholate | 1.97 ++ | 1.23 | 1.62 | 0.46 | | ** | |
| deoxycholate | 2.12 | 1.00 | 0.50 | 0.24 − | ** | | |
| taurodeoxycholate | 2.13 | 1.00 | 0.31 −− | 0.15 −− | ** | | |
| 6-beta-hydroxylithocholate | 1.20 | 1.00 | 0.33 − | 0.27 − | ** | | |
| ursodeoxycholate | 1.56 ++ | 1.00 | 0.16 −− | 0.07 −− | ** | * | * |
| tauroursodeoxycholate | 2.57 ++ | 1.39 | 2.04 ++ | 0.80 | * | ** | * |
| taurohyodeoxycholic acid | 1.68 | 1.00 | 0.27 −− | 0.16 −− | ** | | |
| 3-dehydrocholate | 1.96 + | 1.00 | 0.37 | 0.19 −− | ** | | |
| 7-ketodeoxycholate | 2.04 ++ | 1.00 | 0.2 −− | 0.09 −− | ** | ** | ** |
| ursocholate | 1.24 | 1.00 | 0.4 −− | 0.32 −− | ** | | |
| inosine | 0.14 −− | 2.79 | 0.38 | 7.4 ++ | | | ** |
| hypoxanthine | 0.2 −− | 1.56 | 0.68 | 5.34 ++ | ** | | * |
| xanthine | 0.42 | 1.34 | 0.98 | 3.12 ++ | ** | | |
| urate | 1.16 | 0.86 | 0.91 | 0.67 − | | | |
| allantoin | 1.12 | 1.26 ++ | 1.01 | 1.14 | | ** | |
| allantoic acid | 1.39 | 2.57 ++ | 0.65 | 1.21 | | ** | |
| adenosine 5′-monophosphate (AMP) | 0.77 | 1.20 | 0.68 − | 1.06 | | | |
| adenosine | 0.71 | 1.19 | 1.04 | 1.74 ++ | * | | |
| N1-methyladenosine | 1.31 + | 1.23 | 1.43 + | 1.22 | ** | ** | |
| N6-methyladenosine | 1.30 | 1.41 ++ | 1.00 | 1.09 | | ** | |
| N6-carbamoylthreonyladenosine | 1.20 | 1.36 ++ | 1.00 | 1.14 | | ** | |
| N6-succinyladenosine | 1.44 ++ | 1.57 + | 1.30 | 1.04 | | ** | |
| N2,N2-dimethylguanosine | 1.25 | 0.82 | 0.99 | 0.65 −− | | | |
| N-carbamoylaspartate | 0.21 −− | 0.26 | 0.92 | 1.18 | | ** | |
| orotate | 0.35 −− | 0.45 − | 0.6 − | 0.78 | * | ** | |
| orotidine | 0.96 | 0.86 −− | 1.12 ++ | 1.00 | | ** | |
| uracil | 1.86 ++ | 1.12 | 1.06 | 0.82 | | ** | |
| pseudouridine | 1.29 + | 1.42 + | 1.04 | 1.01 | | ** | |
| 5,6-dihydrouridine | 1.43 | 1.6 + | 0.89 | 1.00 | | ** | |
| 2′-O-methyluridine | 1.02 | 0.79 − | 0.99 | 0.77 −− | | | |
| 5-methyluridine (ribothymidine) | 1.26 | 1.35 ++ | 0.7 −− | 0.75 − | ** | ** | |
| 5,6-dihydrouracil | 5.24 ++ | 1.29 | 1.10 | 0.76 | | ** | |
| 2′-deoxyuridine | 1.4 ++ | 1.58 ++ | 0.83 − | 0.81 −− | ** | ** | |
| 3-ureidoisobutyrate | 1.71 ++ | 3.98 ++ | 1.36 | 1.03 | | ** | |
| 3-ureidopropionate | 1.9 + | 1.54 ++ | 0.97 | 1.17 | | ** | |
| beta-alanine | 0.81 | 1.57 ++ | 0.58 −− | 1.12 | | | ** |
| N-acetyl-beta-alanine | 1.73 ++ | 1.53 + | 1.27 | 1.14 | | ** | |
| cytidine | 1.16 | 1.11 | 1.58 ++ | 1.51 ++ | ** | | |
| 3-methylcytidine | 0.87 | 1.3 + | 0.82 | 1.23 | | | * |
| 5-methylcytidine | 1.42 ++ | 1.14 | 1.36 ++ | 1.10 | ** | ** | |
| N4-acetylcytidine | 1.31 + | 1.71 + | 1.31 | 1.18 | | ** | |
| 2′-deoxycytidine | 2.35 ++ | 1.41 ++ | 1.17 + | 1.16 | | ** | |
| 5-methyl-2′-deoxycytidine | 1.29 + | 1.11 | 1.35 ++ | 1.14 | ** | * | |
| thymidine | 2.64 ++ | 1.76 ++ | 0.90 | 0.92 | | ** | |
| thymine | 3.32 ++ | 2.05 ++ | 1.07 | 0.83 | | ** | |
| 5,6-dihydrothymine | 1.42 ++ | 2.53 ++ | 0.96 | 0.73 | | ** | |
| 3-aminoisobutyrate | 0.69 − | 1.48 | 0.64 | 1.39 | | | * |
| quinolinate | 0.55 −− | 1.88 ++ | 0.57 −− | 1.94 ++ | | | ** |
| nicotinamide | 1.55 ++ | 1.88 ++ | 1.00 | 1.32 ++ | | ** | |
| nicotinamide N-oxide | 2.16 ++ | 2.68 ++ | 1.17 | 1.34 ++ | ** | ** | |
| trigonelline (N′-methylnicotinate) | 1.24 | 2.99 ++ | 0.57 −− | 1.37 | | ** | ** |
| N1-Methyl-2-pyridone-5-carboxamide | 1.34 ++ | 1.55 ++ | 1.06 | 1.06 | | ** | |
| ascorbic acid 2-sulfate | 1.33 | 0.97 | 1.5 ++ | 1.10 | * | | |
| ascorbic acid 3-sulfate* | 1.02 | 0.7 − | 1.89 ++ | 1.30 | ** | | |
| 2-O-methylascorbic acid | 2.3 ++ | 1.25 ++ | 1.10 | 1.02 | | ** | |
| threonate | 1.13 | 1.4 ++ | 1.07 | 1.33 ++ | ** | ** | |
| oxalate (ethanedioate) | 1.43 ++ | 1.62 ++ | 1.83 ++ | 1.37 + | ** | ** | |
| gulonate* | 1.48 ++ | 2.07 ++ | 0.98 | 0.88 | | ** | |
| alpha-tocopherol | 1.21 | 0.63 −− | 1.92 ++ | 1.00 | ** | | ** |
| alpha-CEHC | 0.64 −− | 0.4 −− | 0.66 | 0.42 −− | ** | ** | |
| gamma-tocopherol/beta-tocopherol | 1.10 | 0.7 − | 1.38 | 0.87 | | | |
| biopterin | 1.27 | 0.86 | 1.38 + | 0.93 | | | |
| dihydrobiopterin | 0.93 | 1.11 | 1.05 | 1.26 + | | | |
| heme | 1.24 + | 1.09 | 1.3 ++ | 1.09 | ** | * | |
| biliverdin | 1.57 ++ | 1.08 | 1.14 | 0.83 | | ** | |
| hydroxymethylpyrimidine | 1.18 | 1.00 | 0.43 − | 0.36 | * | | |
| retinol (Vitamin A) | 0.65 −− | 0.79 | 0.74 − | 0.90 | * | ** | |
| pyridoxal | 0.67 − | 0.71 − | 1.02 | 1.08 | | ** | |
| hippurate | 0.21 −− | 0.85 | 0.51 −− | 2.07 ++ | | ** | ** |
| 4-hydroxyhippurate | 0.76 | 0.66 −− | 1.12 | 0.98 | | ** | |
| catechol sulfate | 0.2 −− | 0.29 −− | 0.79 | 1.14 | | ** | |
| 4-ethylcatechol sulfate | 0.15 −− | 0.35 −− | 0.63 | 1.44 | | ** | ** |
| 4-methylcatechol sulfate | 0.31 −− | 0.26 −− | 0.74 | 0.62 | | ** | |
| p-hydroxybenzaldehyde | 0.31 −− | 0.52 | 0.56 −− | 0.95 | | ** | * |
| 4-ethylphenylsulfate | 0.38 −− | 1.32 | 0.52 − | 1.79 ++ | | ** | ** |
| 4-vinylphenol sulfate | 0.3 −− | 1.66 | 0.13 −− | 0.71 | ** | | ** |
| p-cresol sulfate | 0.96 | 0.43 − | 0.28 −− | 0.13 −− | ** | ** | |
| 2-piperidinone | 1.16 | 0.95 | 0.84 | 0.69 − | * | | |
| 2-isopropylmalate | 1.28 | 1.24 | 0.69 −− | 0.67 −− | ** | * | |
| 3-formylindole | 0.46 −− | 0.80 | 0.95 | 1.62 ++ | * | ** | ** |
| gluconate | 1.81 ++ | 1.61 ++ | 0.97 | 0.99 | | ** | |
| dihydrocaffeate sulfate (2) | 1.40 | 1.19 | 0.16 −− | 0.14 −− | ** | | |
| enterolactone sulfate | 0.41 | 0.69 | 0.6 −− | 0.99 | | ** | |
| ferulic acid 4-sulfate | 0.75 | 0.76 | 1.87 + | 1.88 + | ** | | |
| quinate | 1.25 ++ | 1.96 ++ | 1.81 ++ | 2.49 ++ | ** | ** | |
| histidine betaine (hercynine)* | 2.18 | 1.00 | 3.05 ++ | 1.40 | ** | | |
| indolin-2-one | 0.65 −− | 0.97 | 0.2 −− | 0.3 −− | ** | ** | * |
| mannonate* | 0.8 − | 0.57 −− | 1.51 ++ | 1.08 | ** | ** | |
| methyl indole-3-acetate | 0.15 −− | 0.39 −− | 1.04 | 2.69 ++ | ** | ** | |
| stachydrine | 1.56 | 1.46 | 0.33 −− | 0.3 −− | ** | ** | |
| ethyl alpha-glucopyranoside | 0.87 | 2.90 | 0.32 − | 1.08 | | | |
| methyl glucopyranoside (alpha + beta) | 1.15 | 1.82 ++ | 0.61 −− | 0.97 | * | * | |
| 2-keto-3-deoxy-gluconate | 1.14 | 0.7 − | 1.27 | 0.78 | | | * |
| 4-hydroxycinnamate | 0.4 −− | 1.15 | 0.56 | 1.61 | | * | ** |
| caffeic acid sulfate | 0.23 −− | 0.35 −− | 0.65 | 1.00 | | ** | |
| tartronate (hydroxymalonate) | 3.02 ++ | 1.37 + | 1.16 | 0.88 | | ** | |
| 3-indoleglyoxylic acid | 0.31 −− | 0.59 −− | 0.92 | 1.75 ++ | | ** | * |
| ethyl beta-glucopyranoside | 1.22 | 1.36 ++ | 1.06 | 1.18 | | ** | |
| N-methylpipecolate | 1.44 + | 1.3 + | 1.08 | 1.07 | | ** | |
| ampicillin | 1.00 | 13.72 ++ | 3.5 ++ | 48.07 ++ | ** | ** | ** |
| salicylate | 0.85 | 2.14 | 0.90 | 2.27 ++ | | | |
| 2,6-dihydroxybenzoic acid | 0.26 −− | 0.68 | 0.84 | 2.17 ++ | ** | ** | ** |
| neopentyl glycol glutarate** | 1.42 + | 1.11 | 1.56 ++ | 1.22 | ** | | |
| neopentyl glycol adipate** | 1.08 | 1.15 | 1.91 ++ | 2.05 + | ** | | |
| neopentyl glycol suberate** | 1.03 | 0.92 | 2.31 ++ | 2.06 + | ** | | |
| neopentyl glycol azelate** | 0.40 | 0.52 | 2.6 ++ | 3.35 ++ | ** | ** | |
| neopentyl glycol sebacate** | 0.58 | 0.45 −− | 2.49 ++ | 1.92 | ** | ** | |
| neopentyl glycol undecanedioate** | 0.28 | 0.34 −− | 2.86 ++ | 3.49 + | ** | ** | |
| propylene glutarate (1)** | 1.13 | 1.26 | 1.73 + | 1.93 ++ | ** | | |
| propylene glutarate (2)** | 1.13 | 1.30 | 1.76 + | 2.01 ++ | ** | | |
| propylene suberate (1)** | 0.74 | 0.59 | 2.22 ++ | 1.79 | ** | | |
| propylene suberate (2)** | 1.09 | 1.13 | 1.96 ++ | 2.04 + | ** | | |
| sulfate* | 0.91 | 1.08 | 1.03 | 1.22 ++ | * | | |
| S-(3-hydroxypropyl)mercapturic acid | 0.67 −− | 1.11 | 0.83 | 1.39 ++ | | | ** |
| (HPMA) | | | | | | | |
| dimethyl sulfone | 0.64 −− | 0.69 − | 0.78 | 0.84 | * | ** | |
| glycolate (hydroxyacetate) | 3.17 ++ | 1.56 ++ | 1.38 ++ | 0.89 | | ** | ** |
| 2,4-di-tert-butylphenol | 0.80 | 0.29 −− | 1.95 ++ | 0.71 | | ** | ** |
| perfluorooctanesulfonate (PFOS) | 1.81 + | 0.77 − | 1.72 ++ | 0.97 | ** | | ** |
| 6-hydroxyindole sulfate | 1.00 | 1.00 | 0.33 −− | 0.33 | ** | | |
| 4-acetamidobenzoate | 0.45 −− | 2.52 ++ | 0.48 − | 2.69 ++ | | | |
| 3,5-dichloro-2,6-dihydroxybenzoic acid | 0.42 −− | 0.25 −− | 1.6 ++ | 0.97 | ** | ** | * |
| dibutyl sulfosuccinate | 0.61 | 0.55 | 3.99 ++ | 3.61 ++ | ** | | |
| 4-chlorobenzoic acid | 1.14 | 1.02 | 1.2 + | 1.07 | | | |
| perfluorohexanesulfonate (PFHxS) | 3.54 ++ | 1.11 | 1.86 ++ | 1.3 ++ | ** | ** | * |
| glucuronide of C10H18O2 (11)* | 2.55 ++ | 1.62 ++ | 1.19 | 0.64 | | ** | |
| glucuronide of C10H18O2 (12)* | 1.15 | 0.78 | 1.07 | 0.72 − | | | |
| glycine conjugate of C6H10O2 (2)* | 2.95 ++ | 1.52 | 1.35 | 0.80 | | ** | |
| glycine conjugate of C10H14O2 (1)* | 1.9 ++ | 0.74 | 2.84 ++ | 0.90 | ** | | ** |
| branched-chain, straight-chain, or | 0.91 | 0.46 −− | 1.49 + | 0.76 | | ** | ** |
| cyclopropyl 10:1 fatty acid (2)* | | | | | | | |
| bilirubin degradation product, | 1.67 ++ | 1.01 | 0.98 | 0.63 −− | ** | * | * |
| C17H18N2O4 (1)** | | | | | | | |
| bilirubin degradation product, | 1.26 | 0.99 | 0.93 | 0.72 −− | * | | |
| C17H18N2O4 (2)** | | | | | | | |
| bilirubin degradation product, | 1.29 | 0.89 | 0.93 | 0.64 −− | ** | | * |
| C17H18N2O4 (3)** | | | | | | | |
| bilirubin degradation product, | 1.09 | 0.82 | 0.87 | 0.66 − | * | | |
| C17H20N2O5 (2)** | | | | | | | |

SPF, specific pathogen free; CD control diet; PR, protein restricted; ABX, antibiotic.
“++” indicates that mean values are significantly (p < 0.05) higher for that comparison.
“+” indicates that mean values are significantly (0.05 < p < 0.1) higher for that comparison.
“−−” indicates that mean values are significantly (p < 0.05) lower for that comparison.
“−” indicates that mean values are significantly (0.05 < p < 0.1) lower for that comparison.

This work identifies the maternal microbiome as a modifier of adverse neurological outcomes in offspring of PR dams. Maternal PR reduces diversity of the maternal microbiome and elicits widespread alterations in maternal-fetal metabolomic and fetal transcriptomic profiles. These reductions in diversity persist in the immediate postnatal period and correlate with maternal physiological and behavioral shifts, providing alternative routes for further disruption of offspring neurodevelopment, as evidenced by altered anxiety-like and cognitive behaviors in offspring. These findings may have translational implications, as human preterm infants with FGR display a transient early postnatal decrease in microbial diversity which correlates with impaired cognition compared to non-FGR counterparts. Further depleting the maternal microbiome of PR dams throughout gestation substantially alters fetal brain profiles, where only a small fraction of metabolites and genes differentially regulated by maternal microbiome depletion overlap with those altered by PR alone.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of promoting healthy neural development in a fetus, preventing impaired neural development in a fetus, or treating impaired neural development in a fetus, the method comprising administering to a maternal subject gestating the fetus a composition comprising at least one metabolite selected from 3-indole sulfate, phenylacetylglycine, imidazole propionate, alpha-hydroxyisocaproate, 2-hydroxy-3-methylvalerate, alpha-hydroxyisovalerate, 1-methylhistamine, beta-hydroxyisovalerate, 2R,3R-dihydroxybutyrate, and N-acetylleucine, or a combination thereof.

2-3. (canceled)

4. The method of claim 1, wherein the composition comprises 3-indole sulfate.

5. The method of claim 1, wherein the composition comprises phenylacetylglycine.

6. The method of claim 1, wherein the composition comprises imidazole propionate.

7. The method of claim 1, wherein the composition comprises alpha-hydroxyisocaproate.

8. The method of claim 1, wherein the composition comprises 2-hydroxy-3-methylvalerate.

9. The method of claim 1, wherein the composition comprises alpha-hydroxyisovalerate.

10. The method of claim 1, wherein the composition comprises 1-methylhistamine.

11. The method of claim 1, wherein the composition comprises beta-hydroxyisovalerate.

12. The method of claim 1, wherein the composition comprises 2R,3R-dihydroxybutyrate.

13. The method of claim 1, wherein the composition comprises N-acetylleucine.

14. A method of promoting healthy neural development in a fetus, preventing impaired neural development in a fetus, or treating impaired neural development in a fetus, the method comprising administering to a maternal subject gestating the fetus a composition comprising at least one short chain fatty acid selected from acetate, butyrate, and propionate, or a combination thereof.

15-16. (canceled)

17. The method of claim 14, wherein the composition comprises acetate.

18. The method of claim 14, wherein the composition comprises butyrate.

19. The method of claim 14, wherein the composition comprises propionate.

20. The method of claim 14, wherein the maternal subject is undernourished due to at least one of protein deficiency, poor weight gain, and fetal growth restriction.

21-23. (canceled)

24. The method of claim 14, wherein the method comprises administering the composition:
(a) at least once during the first trimester of the maternal subject's gestation period,
(b) at least once during a period that runs from the start of the third week after conception to the end of the eighth week after conception,
(c) at least once during a period that runs from the 17^{th} day post conception (dpc) to the 52^{nd} dpc,
(d) at least once during the second trimester of the maternal subject's gestation period, or
(e) at least once during the third trimester of the maternal subject's gestation period.

25-28. (canceled)

29. The method of claim 14, wherein the fetus is an offspring of the maternal subject.

30. A method of conditioning a female subject for fostering healthy neural development in offspring, the method comprising administering to the female subject;
(a) a composition comprising at least one metabolite selected from 3-indole sulfate, phenylacetylglycine, imidazole propionate, alpha-hydroxyisocaproate, 2-hydroxy-3-methylvalerate, alpha-hydroxyisovalerate, 1-methylhistamine, beta-hydroxyisovalerate, 2R,3R-dihydroxybutyrate, and N-acetylleucine, or a combination thereof, or
(b) a composition comprising at least one short chain fatty acid selected from acetate, butyrate, and propionate, or a combination thereof; wherein the composition is administered at least once during a period that runs from the first day of an expected-but-missed menstruation to the end of gestation.

31. (canceled)

32. The method of claim 30, wherein healthy neural development comprises a reduction in anxiety-like behavioral deficits in the offspring or prevention of learning and memory deficits.

33. (canceled)
